(19) United States
(12) Patent Application Publication
(10) Pub. No.: US 2025/0360268 A1
(43) Pub. Date: Nov. 27, 2025

(54) Syringe Pump Apparatuses, Systems, and Methods

(71) Applicant: DEKA Products Limited Partnership, Manchester, NH (US)

(72) Inventors: Keith D. Violette, Sandown, NH (US); Nicholas A. Bealo, Deerfield, NH (US); Alexander C. Bisson, Manchester, NH (US); Ryan P. Wood, Bedford, NH (US); Trevor A. Conway, Manchester, NH (US); Matthew R. Anderson, Manchester, NH (US); David Blumberg, JR., Epsom, NH (US); Gregory N. Lerner, Pembroke, NH (US); Brandon W. Zalinsky, Pembroke, NH (US); Robert Piccirillo, Salem, NH (US); David D.B. Cannan, Manchester, NH (US); Alden M. Shoup, Towson, MD (US)

(21) Appl. No.: 19/206,681

(22) Filed: May 13, 2025

Related U.S. Application Data
(60) Provisional application No. 63/646,164, filed on May 13, 2024.

(51) Int. Cl. A61M 5/145; A61M 5/172

(52) U.S. Cl. CPC A61M 5/1452; A61M 5/172; A61M 2205/10; A61M 2205/3327; A61M 2205/50

(57) ABSTRACT

A syringe pump may comprise a main housing. The syringe pump may further comprise a drive head coupled to a drive head tube and displaceable relative to the main housing. The drive head may include a means for positioning and retaining a plunger flange of a plunger of a syringe in alignment with a fiducial reference axis. The syringe pump may further comprise a means for positioning and retaining a barrel of the syringe in alignment with the fiducial reference axis. The syringe pump may further comprise a load cell assembly including a load cell and a contact in mechanical communication with the load cell. The contact may be disposed on the drive head and in alignment with the fiducial reference axis.

### CROSS-REFERENCE TO RELATED APPLICATIONS

This application claims the benefit of U.S. Provisional Patent Application No. 63/646,164, filed on May 13, 2024, which is incorporated herein by reference in its entirety.

### BACKGROUND

### Field of Disclosure

This disclosure relates to delivery of fluids. More specifically, this disclosure relates to delivery of medical fluids to patients with pumps.

### Description of Related Art

Syringes pumps are used in a variety of medical applications, such as intravenous delivery of medications, for example to a patient in an intensive-care unit. Syringe pumps may be designed so that needles, tubing, or other attachments can be coupled to the syringe pump. Syringe pumps are typically used with a reservoir which may be a tube shaped structure having a port at one end. A plunger may be driven into the reservoir such that the plunger pushes fluid out of the syringe pump. Syringe pumps may include an actuator which mechanically drives the plunger to control discharge of fluid from the reservoir. Syringe pumps may be used to deliver various drugs including analgesics, antiemetics, antibiotics, or a variety of other agents. Agents may be administered via an intravenous liquid line quick (e.g. in a bolus) or over a longer length of time. Syringe pumps may also be used in non-medical applications such as in laboratory testing, in microreactors, and/or in chemical processing applications.

### SUMMARY

In accordance with an example embodiment of the present disclosure an example drive head for a syringe pump may comprise a housing. The drive head may further comprise a set of plunger flange grasper bodies displaceable about respective pivot axes. The drive head may further comprise a plurality of identical planetary gears. Each plunger flange grasper body may be coupled to one of the plurality of planetary gears. The drive head may further comprise a sun gear in interdigitating relationship with the plurality of planetary gear and rotatable about an axis centrally disposed with respect to the respective pivot axes. The sun gear may include a slot. The drive head may further comprise an actuator coupled to a linkage having a projection disposed within the slot. Displacement of the actuator from a home position to an actuated position may be transmitted through the sun gear and planetary gears to displace each plunger flange grasper body from a closed position to an open position. The drive head may further comprise at least one first bias member urging the plunger flange grasper bodies to the closed position. The drive head may further comprise a second bias member urging the actuator to the home position. The projection may be configured to travel along the slot when the second bias member is driving the actuator to the home position and the plunger flange grasper bodies are blocked from returning to the fully closed position.

In accordance with another example embodiment of the present disclosure an example drive head for a syringe pump may comprise a housing. The drive head may further comprise a set of plunger flange grasper bodies displaceable about respective pivot axes. The drive head may further comprise a plurality of identical planetary gears. Each plunger flange grasper body may be coupled to one of the plurality of planetary gears. The drive head may further comprise a sun gear in interdigitating relationship with the plurality of planetary gear and rotatable about an axis centrally disposed with respect to the respective pivot axes. The sun gear may include a slot. The drive head may further comprise an actuator coupled to the sun gear via a linkage having a projection disposed within the slot. Displacement of the actuator from a home position to an actuated position may be transmitted through the sun gear and planetary gears to displace each plunger flange grasper body from a closed position to an open position.

In some embodiments, the drive head may further comprise at least one first bias member urging the plunger flange grasper bodies to the closed position. In some embodiments, the drive head may further comprise at least one second bias member urging the actuator to the home position. The slot may be configured to provide a clearance for the projection sufficient to allow the second bias member to drive the actuator to the home position when the plunger flange grasper bodies are blocked from displacing fully to the closed position. In some embodiments, the plunger flange grasper bodies may be configured to center plunger flanges of a variety of diameters along a fiducial reference axis. In some embodiments, the drive head may further comprise a load cell disposed in line with the fiducial reference axis. In some embodiments, each of the plunger flange grasper bodies may include a snugging face. In some embodiments, three plunger flange grasper bodies are included in the set of plunger flange grasper bodies.

In accordance with another example embodiment of the present disclosure an example method of retaining a plunger flange of a syringe against a drive head of a syringe pump may comprise displacing an actuator of the drive head. The method may further comprise transmitting displacement of the actuator to a sun gear of the drive head to rotate the sun gear. The method may further comprise rotating a set of planetary gears interdigitating with the sun gear. The method may further comprise rotating a set of plunger flange grasper bodies each coupled to a respective planetary gear of the set of planetary gears from a closed position to an open position. The method may further comprise placing a plunger flange within a region through which a fiducial reference axis passes. The method may further comprise centering the plunger flange on the fiducial reference axis via displacement of plunger flange grasper bodies against the plunger flange from the open position.

In some embodiments, each of the plunger flange grasper bodies may include a snugging face. Centering the plunger flange on the fiducial reference axis via displacement of the plunger flange grasper bodies against the plunger flange may further comprise pressing the plunger flange against the drive head. In some embodiments, each of the plunger flange grasper bodies may include a snugging face. Centering the plunger flange on the fiducial reference axis via displacement of the plunger flange grasper bodies against the plunger flange may further comprise pressing the plunger flange against a contact for a load cell disposed in line with the fiducial reference axis. In some embodiments, the method may further comprise disengaging a clutch operatively coupled to the leadscrew via displacement of the actuator. In some embodiments, transmitting displacement of the actuator to the sun gear may comprise driving a pin of a linkage coupled to the actuator against an end of a slot included in the sun gear. In some embodiments, the method may further comprise driving the actuator to a home position with at least one actuator bias member and providing a clearance in the slot sufficient to allow the pin freely displace at least until the actuator reaches the home position when the plunger flange grasper bodies are against the plunger flange. In some embodiments, the method may further comprise biasing, with at least one actuator bias member, the actuator to a home position. In some embodiments, the method may further comprise biasing, with at least one grasper bias member, each of the plunger flange grasper bodies toward the closed position.

In accordance with another example embodiment of the present disclosure an example drive head for a syringe pump may comprise a housing. The drive head may further comprise a first plunger flange grasper body pivotally displaceable about a first pivot axis. The drive head may further comprise a second plunger flange grasper body pivotally displaceable about a second pivot axis and geared to the first plunger flange grasper body such that displacement of the first plunger flange grasper body in a first pivotal direction engenders a pivotal displacement of the second plunger flange grasper body in an opposite, second pivotal direction. The drive head may further comprise an actuator coupled to the first plunger flange grasper via a pivot body and a linkage with a projection. The pivot body may include a slot in which the projection is disposed. Displacement of the actuator from a home position to an actuated position may be transmitted through the linkage and pivot body to displace each plunger flange grasper body from a closed position to an open position. The drive head may further comprise a first bias member urging the plunger flange grasper bodies to the closed position. The drive head may further comprise a second bias member urging the actuator to the home position. The slot may be configured to provide a clearance for the projection sufficient to allow the second bias member to drive the actuator to the home position when the plunger flange grasper bodies are blocked from displacing fully to the closed position. The shapes of the first and second plunger flange grasper bodies may be configured to center a plunger flange of an installed syringe on a fixed fiducial reference axis when the plunger flange is within an accepted size range.

In accordance with another example embodiment of the present disclosure an example drive head for a syringe pump may comprise a housing. The drive head may further comprise a first plunger flange grasper body displaceable about a first pivot axis. The drive head may further comprise a second plunger flange grasper body displaceable about a second pivot axis and geared to the first plunger flange grasper body such that rotation of the first plunger flange grasper body in a first direction engenders rotation of the second plunger flange grasper body in an opposite direction. The drive head may further comprise an actuator coupled to the first plunger flange grasper via a transmission. Displacement of the actuator between a home position and an actuated position may be transmitted through the transmission to displace the first and second plunger flange grasper bodies. The drive head may further comprise a contact body in mechanical communication with a load cell disposed in the housing. The shapes of the first and second plunger flange grasper bodies may be configured to center a plunger flange of an installed syringe on a fixed fiducial reference axis extending through a center of the contact body when the plunger flange is within an accepted size range.

In accordance with another example embodiment of the present disclosure an example drive head for a syringe pump may comprise a housing. The drive head may further comprise a first plunger flange grasper body displaceable about a first pivot axis. The drive head may further comprise a second plunger flange grasper body displaceable about a second pivot axis and geared to the first plunger flange grasper body such that rotation of the first plunger flange grasper body in a first direction engenders rotation of the second plunger flange grasper body in an opposite direction. The drive head may further comprise an actuator coupled to the first plunger flange grasper via a transmission. Displacement of the actuator between a home position and an actuated position may be transmitted through the transmission to displace the first and second plunger flange grasper bodies. The shapes of the first and second plunger flange grasper bodies may be configured to center a plunger flange of an installed syringe on a fixed fiducial reference axis when the plunger flange is within an accepted size range.

In some embodiments, the drive head may further include a load cell and a load cell plunger flange contact configured to communicate force exerted on the load cell plunger flange contact to the load cell. In some embodiments, the fiducial reference axis may extend through the center of the load cell plunger flange contact. In some embodiments, the first and second plunger flange grasper bodies may include snugging faces configured to press the plunger flange of the installed syringe against the load cell plunger flange contact. In some embodiments, the first and second plunger flange grasper bodies may be configured to hold the plunger flange of the installed syringe against an exterior surface of the drive head. In some embodiments, the drive head may further comprise a clutch assembly. The actuator may be configured to displace a pressure plate of the clutch from a clutch engaging state to a clutch disengaging state as the actuator is displaced from the home position to the actuated position. In some embodiments, the drive head may further comprise a first bias member configured to urge the plunger flange grasper bodies against one another. In some embodiments, the drive head may further comprise an actuator bias member configured to urge the actuator to the home position. In some embodiments, the transmission may include a pivot body and a linkage. The pivot body may include a slot into which a projection of the linkage extends. The slot may be configured to provide a clearance for the projection sufficient to allow the second bias member to drive the actuator to the home position absent corresponding displacement of the plunger flange grasper bodies.

In accordance with another example embodiment of the present disclosure an exemplary method of retaining a plunger flange of a syringe against a drive head of a syringe pump may comprise displacing an actuator of the drive head. The method may further comprise transmitting displacement of the actuator to first and second plunger flange grasper bodies to rotate the first and second plunger flange grasper bodies from a closed position to an open position. The method may further comprise placing a plunger flange within a region through which a fiducial reference axis passes. The method may further comprise centering the plunger flange on the fiducial reference axis by displacing the first and second plunger flange grasper body into contact with the plunger flange such that the first plunger flange grasper body contacts the plunger flange at substantially a single point and the second plunger flange grasper body contacts the plunger flange at substantially two separate points.

In some embodiments, each of the plunger flange grasper bodies may include a snugging face. Centering the plunger flange on the fiducial reference axis via displacement of the plunger flange grasper bodies against the plunger flange may further comprise pressing the plunger flange against the drive head. In some embodiments, each of the plunger flange grasper bodies may include a snugging face. Centering the plunger flange on the fiducial reference axis via displacement of the plunger flange grasper bodies against the plunger flange may further comprise pressing the plunger flange against a contact for a load cell disposed in line with the fiducial reference axis. In some embodiments, placing the plunger flange within the region through which the fiducial reference axis passes may comprise placing the plunger flange against a contact for a load cell disposed in the drive head. In some embodiments, the method may further comprise disengaging a clutch operatively coupled to the leadscrew via displacement of the actuator. In some embodiments, transmitting displacement of the actuator to the first and second plunger flange grasper bodies may comprise driving a pin of a linkage coupled to the actuator against a sidewall of a slot in pivot body coupled to one of the first and second plunger flange grasper bodies. In some embodiments, the method may further comprise driving the actuator to a home position with at least one actuator bias member and providing a clearance in the slot sufficient to allow the pin freely displace at least until the actuator reaches the home position when the plunger flange grasper bodies are against the plunger flange. In some embodiments, the method may further comprise biasing, with at least one actuator bias member, the actuator to a home position. In some embodiments, the method may further comprise biasing, with at least one grasper bias member, each of the plunger flange grasper bodies toward the closed position.

In accordance with another example embodiment of the present disclosure an exemplary method of retaining a plunger flange of a syringe against a drive head of a syringe pump may comprise displacing an actuator of the drive head. The method may further comprise transmitting displacement of the actuator to at least first and second plunger flange grasper bodies to rotate the at least first and second plunger flange grasper bodies from a closed position to an open position. The method may further comprise placing a plunger flange within a region through which a fiducial reference axis passes. The method may further comprise centering the plunger flange on the fiducial reference axis via displacement of plunger flange grasper bodies against the plunger flange from the open position.

In accordance with an example embodiment of the present disclosure an exemplary drive head for a syringe pump may comprise a housing having a drive head tube extending from the housing and being coupled to the housing. The drive head may further comprise a floating portion displaceable with respect to the housing. The floating portion my comprise a load cell assembly having a beam coupled to the housing. The beam may include a flexure. The load cell assembly may further include a load cell configured to output a signal relative to an amount of deflection of the flexure. The floating portion further comprising a set of plunger flange graspers coupled to the beam. The floating portion may further comprise a first panel coupled to the beam. The drive head may further comprise a sealing member forming a fluid tight seal between the housing and the floating portion.

In some embodiments, the panel may include a raised plateau for contacting the plunger flange of a syringe. In some embodiments, the set of plunger flange graspers may be mounted to the panel and coupled to the beam through the panel. The plunger flange graspers may be rotatable relative to the panel. In some embodiments, the set of plunger flange graspers may be configured to center a plunger flange of a syringe on a fiducial reference axis. In some embodiments, the floating portion may further comprise a sensor circuit board having a plunger flange rotary position sensor disposed thereon. In some embodiments, the set of plunger flange graspers may include a metallic body and the plunger flange rotary position sensor may be configured to generate an output signal which varies in relation to the location of the metallic body relative to the sensor. In some embodiments, the drive head may further comprise a flex cable extending from the sensor circuit board and through the drive head tube. In some embodiments, the floating assembly may include a second panel coupled to the first panel. A portion of the scaling member may be captured and compressed between the first and second panel. In some embodiments, the sealing member may provide a diaphragm spanning a gap between the periphery of the first and second panels and a surround portion of the housing. In some embodiments, the drive head may further comprise a tray. Portions of the sealing member may be captured and compressed between the tray and an interior surface of the housing.

In accordance with another example embodiment of the present disclosure an example drive head for a syringe pump may comprise a housing having a drive head tube extending from the housing and being coupled to the housing. The drive head may further comprise a floating portion displaceable with respect to the housing. The floating portion may comprise a load cell assembly including a beam with a flexure. The floating portion may further comprise a load cell configured to output a signal which varies in relation to an amount of deflection of the flexure. The floating assembly may be coupled to the housing via the load cell assembly. The floating portion may further comprise a set of plunger flange graspers coupled to the beam. The floating portion may further comprise a first panel coupled to the beam. The drive head may further comprise a sealing member forming a fluid tight seal between the housing and the floating portion.

In accordance with an example embodiment of the present disclosure an exemplary method of collecting data relating to a delivery pressure inside a syringe during an infusion therapy may comprise providing a syringe pump with a displaceable drive head having a floating assembly displaceable relative to a remainder of the drive head. The floating assembly may comprise a load cell assembly coupled to the remainder of the drive head and a set of plunger flange grasper bodies. The method may further comprise holding the syringe barrel in fixed position relative to a housing of the syringe pump. The method may further comprise capturing a plunger flange of a plunger of the syringe against the drive head by displacing the set of plunger flange grasper bodies into the plunger flange and snugging the plunger flange into abutment with a portion of the floating assembly. The method may further comprise displacing the drive head to drive the plunger into the barrel of the syringe. The method may further comprise receiving a signal from the load cell assembly with a controller of the syringe pump.

In some embodiments, the method may further comprise sealing the drive head such that the interior of the drive head is isolated from the surrounding environment when the floating assembly is displaced through a floating assembly displacement range. In some embodiments, holding the syringe barrel in fixed position relative to the housing may comprise capturing the syringe barrel in a barrel clamp and aligning the syringe barrel along a fiducial reference axis. In some embodiments, the method may further comprise capturing a barrel flange of the syringe in a barrel flange clamp. In some embodiments, capturing the plunger flange of the syringe against the drive head may further comprise centering the plunger flange on a fiducial reference axis. In some embodiments, the method may further comprise generating, with the controller, a notification that the signal from the load cell assembly is in breach of the threshold. In some embodiments, the load cell assembly may include a beam cantilevered from the remainder of the drive head. In some embodiments, the method may further comprise sensing a position of at least one of the set of the plunger flange grasper bodies with a sensor disposed on a sensor circuit board included in the floating assembly. In some embodiments, displacing the drive head may comprise translating the drive head along a first portion of a rigid guide extending from the housing and translating a carriage along a second portion of the rigid guide disposed within the housing. The drive head and carriage may be at a fixed distance from one another.

In accordance with another example embodiment of the present disclosure an example syringe pump may comprise a main housing. The syringe pump may further comprise a drive head coupled to a drive head tube and displaceable relative to the main housing. The drive head may comprise a means for position and retaining a plunger flange of a plunger of a syringe in alignment with a fiducial reference axis. The syringe pump may further comprise a means for positioning and retaining a barrel of the syringe in alignment with the fiducial reference axis. The syringe pump may further comprise a load cell assembly including a load cell and a contact in mechanical communication with the load cell. The contact may be disposed on the drive head and in alignment with the fiducial reference axis.

In some embodiments, the syringe pump may further comprise a flange clip configured to retain a barrel flange of the syringe in place relative to the main housing. In some embodiments, the syringe pump may further comprise a leadscrew and a clutch assembly. The clutch assembly may have an engaged state in which the leadscrew is inhibited from rotating about an axis of the leadscrew and a disengaged state in which the leadscrew is free to rotate about the axis of the leadscrew. In some embodiments, the drive head may include an actuator configured to transition the clutch assembly between the engaged and disengaged states when the actuator is displaced from a resting position to an actuated position. In some embodiments, the syringe pump may further comprise a leadscrew and a drive motor coupled to a nut tube having a nut coupled thereto. The nut may be concentric with the nut tube. The nut may be in permanent engagement with the nut tube. In some embodiments, the syringe pump may further comprise a clutch assembly in the drive head. The leadscrew may be operatively engaged with the clutch assembly. In some embodiments, the syringe pump may further comprise a rigid guide. The drive head may include a bearing portion configured to ride along the rigid guide as the drive head is displaced relative to the main housing. In some embodiments, a carriage may be coupled to an end region of the drive head tube opposite the drive head. The carriage may include a bearing portion configured to displace along the rigid guide as the drive head is displaced relative to the main housing.

In accordance with another example embodiment of the present disclosure an example syringe pump may comprise a main housing. The syringe pump may further comprise a drive head coupled to a drive head tube and displaceable relative to the main housing. They syringe pump may further comprise a set of plunger flange grasper bodies coupled to the drive head. The syringe pump may further comprise a barrel grasper assembly including a first barrel contacting body and a second barrel contacting body. The first barrel contacting body and second barrel contacting body may be coupled to one another via a linking assembly such that movement of one of the first barrel contacting body engenders a corresponding displacement of the second barrel contacting body. Each of the barrel contacting bodies may include a contact face shaped to center a barrel of a syringe within an accepted size range on a fiducial reference axis.

In some embodiments, the syringe pump may further comprise a sensing assembly. The sensing assembly may be configured to output a signal which varies in relation to the position of one of the first and second barrel contacting bodies. In some embodiments, the syringe pump may further comprise a controller configured to determine an identity of a retained syringe in the barrel grasper assembly based at least in part on the signal. In some embodiments, the linking assembly may include a set of gears. In some embodiments, the linking assembly may include one or more linkage. In some embodiments, the first and second barrel contacting bodies may be cam jaws including a cam face. The first and second barrel contacting bodies may be pivotally coupled to the syringe pump. In some embodiments, the barrel contacting face of the first barrel contacting body may include a single barrel contact point at a tip region of the barrel contacting body and the barrel contacting face of the second barrel contacting body may include a first and second barrel contact point. The first contact point may be disposed on a convexly rounded portion of the barrel contacting face. The second contact point may be disposed on a concavely rounded portion of the barrel contacting face and more proximate a tip of the second barrel contacting body than the first contact point. In some embodiments, the plunger flange grasper bodies may be displaceable between an open and closed position. The plunger flange grasper bodies may be configured to center a plunger flange of a syringe within the accepted size range on the fiducial reference axis when displaced toward the closed position against the plunger flange. In some embodiments, the drive head may further comprise a load cell assembly having a load cell and a contact in mechanical communication with the load cell. The contact may be aligned with the fiducial reference axis. In some embodiments, the plunger flange grasper bodies may be configured to hold the plunger flange against the contact. In some embodiments, the syringe pump may further comprise a barrel flange clip configured to retain a barrel flange of a syringe. In some embodiments, the barrel flange clip is coupled to the barrel grasper assembly. In some embodiments, the syringe pump may further comprise a barrel flange clip sensor configured to output a barrel flange clip sensor signal which varies in relation to displacement of the barrel flange clip. In some embodiments, the barrel grasper assembly may further comprise at least one bias member configured to urge the barrel contacting bodies toward a closed state.

In accordance with another example embodiment of the present disclosure an example syringe pump may comprise a main housing. The syringe pump may further comprise a drive head coupled to a drive head tube and displaceable relative to the main housing. The syringe pump may further comprise a set of plunger flange grasper bodies coupled to the drive head. The syringe pump may further comprise a barrel grasper assembly including a first barrel retainer body, a second barrel retainer body, and a linking assembly configured to engender displacement of the second barrel retainer body when the first barrel retainer body is displaced. The barrel retainer bodies may be configured to center a barrel of a syringe within an accepted size range on a fiducial reference axis.

In some embodiments, the syringe pump may further comprise a sensing assembly configured to output a signal indicative of the position of one of the first and second barrel retainer bodies. In some embodiments, the syringe pump may further comprise a controller configured to determine an identity of a retained syringe in the barrel grasper assembly based at least in part on the signal. In some embodiments, the linking assembly may include a set of gears. In some embodiments, the linking assembly may include one or more linkage. In some embodiments, the first and second barrel retaining bodies may be cam jaws including a cam face. The first and second barrel contacting bodies may be pivotally coupled to the syringe pump. In some embodiments, the first barrel retainer body may include a single barrel contact point at a tip region of the first barrel retainer body and the second barrel retainer body may include a first and second barrel contact point disposed respectively on a convexly and concavely rounded surface of the second barrel retainer body. The second barrel contact point may be more proximate a tip of the second barrel retainer body than the first barrel contact point. In some embodiments, the plunger flange grasper bodies may be configured to center a plunger flange of a syringe within the accepted size range on the fiducial reference axis when displaced from an open position toward a closed position and into contact with the plunger flange. In some embodiments, the drive head may further comprise a load cell assembly having a load cell and a contact in mechanical communication with the load cell. The contact being aligned with the fiducial reference axis. In some embodiments, the plunger flange grasper bodies may be configured to hold the plunger flange against the contact. In some embodiments, the syringe pump may further comprise a barrel flange clip configured to retain a barrel flange of a syringe. In some embodiments, the barrel flange clip may be coupled to the main housing. In some embodiments, the syringe pump may further comprise a barrel flange clip sensor configured to output a barrel flange clip sensor signal which varies in relation to displacement of a clip member of the barrel flange clip. In some embodiments, the barrel grasper assembly may further comprise at least one bias member configured to urge the barrel retainer bodies toward a closed state.

In accordance with another exemplary embodiment of the present disclosure an example method of retaining a syringe with any barrel diameter in an accepted size range in place relative to a syringe pump may comprise displacing a first barrel retainer body and a second barrel retainer body relative to one another from a closed state to an open state. The method may further comprise coordinating relative motion of the first and second barrel retainer body with a linking assembly. The method may further comprise placing a barrel of the syringe between the first and second barrel retainer body. The method may further comprise centering the barrel on a fiducial reference axis via displacement of the first and second barrel retainer body against the barrel from the open state toward the closed state.

In some embodiments, the method may further comprise sensing a position, via a sensing assembly, of at least one of the first and second barrel retainer bodies. In some embodiments, the method may further comprise determining, via a controller of the syringe pump, an identity of the syringe based at least in part on the position sensed by the sensing assembly. In some embodiments, the method may further comprise capturing a barrel flange of the barrel in a barrel flange clip. In some embodiments, the method further may comprise sensing a position of a clip portion of the barrel flange clip and determining, via a controller of the syringe pump, presence of a barrel flange in the barrel flange clip based at least in part on the sensed position of the clip portion. In some embodiments, centering the barrel on the fiducial reference axis via displacement of the first and second barrel retainer body may comprise contacting the barrel with the first retainer body at a single contact point at a tip region of the first retainer body and contacting the barrel with the second retainer body at a first and second contact point respectively disposed on a convexly and concavely rounded portion of the second retainer body. The second contact point may be more proximal the tip of the second retainer body than the first contact point. In some embodiments, the first and second barrel retainer body may be cam jaws and displacing the first and second barrel retainer bodies may comprise pivoting the first and second barrel retainer bodies about respective axes. In some embodiments coordinating relative motion of the first and second barrel retainer bodies may comprise transmitting motion through a set of gears. In some embodiments, coordinating relative motion of the first and second barrel retainer bodies may comprise transmitting motion through a linkage arrangement coupled to each of the first and second barrel retainer bodies.

In accordance with another example embodiment of the present disclosure an example syringe pump for dispensing fluid from a syringe may comprise a main housing including a guide assembly. The syringe pump may further comprise a drive head including a bearing portion configured to ride along the guide assembly. The syringe pump may further comprise a syringe barrel mounting assembly coupled to the main housing. The syringe pump may further comprise a syringe plunger grasping assembly coupled to the drive head. The syringe pump may further comprise a drive assembly. The drive assembly may comprise a drive motor. The drive assembly may further comprise a transmission. The drive assembly may further comprise a nut. The drive assembly may further comprise a first tube coupled to the transmission and the nut and being disposed substantially concentric with the nut. The drive assembly may further comprise a second tube coupled to the drive head at one end and a carriage at the opposing end. The carriage may be configured to ride along the guide assembly. The drive assembly may further comprise a lead screw extending from the drive head, through the second tube and into permanent engagement with the nut.

In some embodiments, the transmission may be non-backdriveable. In some embodiments, the transmission may include a worm gear. In some embodiments, the pitch of the leadscrew may be at least 10 mm. In some embodiments, the leadscrew may include at least 3 thread starts. In some embodiments, the syringe pump may further comprise a clutch and an actuator. The clutch may be engaged with the leadscrew when the actuator is in a first position and disengaged with the leadscrew when the actuator is in a second position. In some embodiments, the actuator may be coupled to the drive head and include a lever. The actuator may be biased to the first position. In some embodiments, the plunger flange grasping assembly may include a set of rotatable arms. The actuator may be coupled to the set of rotatable arms via at least one gear. In some embodiments, the plunger flange grasping assembly may include a set of rotatable arms. The actuator may be coupled to the set of rotatable arms via a linkage coupled to a pivoting body. Transition of the actuator from the first position to the second position may displace the linkage and engender rotation of the pivoting body. In some embodiments, the rotatable arms may be biased toward a closed position by an arm bias member and the actuator may be biased to the first position by an actuator bias member. The linkage may be coupled to the pivot body by a projection extending into a slot of the pivot body. The projection may displace along the slot without engendering displacement of the pivot body when the rotatable arms are blocked from returning to the closed position and the actuator bias member urges the actuator from the second position to the first position. In some embodiments, the first tube may be sized to fit within the second tube.

In accordance with another example embodiment of the present disclosure a drive assembly for a syringe pump may comprise a guide assembly. The syringe pump may further comprise a driving portion including a first tube having a nut coupled thereto. The nut may be concentric with the first tube. The syringe pump may further comprise a motor and transmission coupled to the first tube and configured to rotationally displace the first tube. The syringe pump may further comprise a driven portion including a second tube sized to telescope over the first tube. The second tube may have a carriage coupled to a first end and a drive head including a set of plunger flange graspers coupled to a second opposing end. The drive head and carriage may be configured to displace along at least one bearing surface of the guide assembly. The driven portion may further have a leadscrew extending through the second tube and into permanent engagement with the nut.

In some embodiments, the nut may include at least three thread starts. In some embodiments, the leadscrew may have a pitch of at least 10 mm. In some embodiments, the nut may be disposed at an end of the first tube. In some embodiments, the transmission may include a worm gear. In some embodiments, the motor and transmission may be non-backdriveable. In some embodiments, the drive assembly may further comprise a clutch and an actuator. The actuator may be displaceable between a first position to a second position to respectively transition the clutch between a first state in which it is engaged with the leadscrew and a second state in which it is disengaged with the leadscrew. In some embodiments, the actuator may be biased to the first position. In some embodiments, the actuator may further be operatively coupled to the plunger flange graspers. The plunger flange graspers may transition from a spread state to a closed state as the actuator is displaced from the second position to the first position.

In accordance with an example embodiment of the present disclosure an exemplary method of dispensing fluid from a syringe associated with a syringe pump may comprise powering a motor. The method may further comprise transmitting an output of the motor to a driving portion of a drive assembly of the syringe pump to engender rotation of a nut coupled to a first elongate body. The method may further comprise advancing a leadscrew of the drive assembly into the first elongate body. The leadscrew may be coupled to a drive head of the syringe pump. The method may further comprise displacing a carriage attached to a second elongate body at an end region of the second elongate body most distal to the drive head along a rigid guide. The method may further comprise displacing a bearing portion of the drive head along the rigid guide. The method may further comprise driving a plunger of the syringe into a barrel of the syringe as the drive head is displacing along the rigid guide.

In some embodiments, the method may further comprise engaging a clutch in the drive head. The leadscrew may be inhibited from rotation about an axis of the leadscrew when the clutch is engaged. In some embodiments, transmitting the output of the motor to the driving portion may comprise rotating a worm gear. In some embodiments, transmitting the output of the motor to the driving portion may comprise transmitting the output of the motor through a worm gear to a worm wheel coupled to the first elongate body. In some embodiments, the method may further comprise sensing a position of the carriage along the rigid guide. In some embodiments, displacing the carriage attached to the second elongate body may comprise telescoping the second elongate body over the first elongate body. In some embodiments, the leadscrew may have a thread pitch of at least 10 mm and at least three thread starts.

In accordance with another example embodiment of the present disclosure an example drive assembly for a medical pump may comprise a leadscrew extending along an axis. The drive assembly may comprise a motor operatively coupled to the leadscrew configured to generate rotational displacement of the leadscrew. The drive assembly may further comprise a nut assembly disposed substantially coaxially with the leadscrew and through which the leadscrew extends. The nut assembly may comprise a stationary portion having an arm connected thereto. The arm may be displaceable with respect to the stationary portion and include an interface surface and a threaded region configured to engage threads of the leadscrew. The nut assembly may further comprise an actuation subassembly including at least one guide surface. The nut assembly may further comprise an actuator coupled to the actuation subassembly and displaceable to translationally drive the actuation subassembly between a first position and second position relative to the stationary portion. The nut assembly may further comprise at least one bias member configured to urge the actuation subassembly toward one of the first and second positions. When the actuation subassembly is displaced with respect to the stationary portion from the first position toward the second position, the guide surface may press the threaded region of the arm into engagement with the leadscrew via interaction with the interface surface.

In some embodiments, the arm may be integrally formed with the stationary portion and deflectable with respect to the stationary portion. In some embodiments, the arm may be coupled to the stationary portion via a pivot pin. The arm may be pivotable about the axis of the pivot pin. In some embodiments, the arm includes a projection. The interface surface may be defined on the projection. In some embodiments, the actuation subassembly may include a body disposed in flanking relation to the arm. The body may include a track into which the first projection extends. The sidewalls of the track may define the at least one guide surface. In some embodiments, the interface surface may be a face of the arm disposed on a side of the arm opposite the threaded region. In some embodiments, the actuation subassembly may include a main body having an aperture through which the leadscrew extends. The aperture may including a taper along at least a portion of the wall of the aperture. The taper may define the guide surface. In some embodiments, the at least one bias member may be configured to urge the actuation subassembly toward the second position and the arm may be coupled to a torsion spring configured to urge the threaded region of the arm away from the leadscrew. In some embodiments, the actuator may include at least one wire coupled to the actuation subassembly. In some embodiments, the stationary portion of the nut assembly may include a housing which surrounds at least a portion of the actuation subassembly. In some embodiments the leadscrew may include at least three thread starts and has a pitch of at least 10 mm. In some embodiments, the interface surface may be shy of an end of the guide surface when the threaded portion of the arm is in engagement with the leadscrew. In some embodiments, when the threaded portion of the arm is in engagement with the leadscrew, the interface surface may present an interference which prevents displacement of the actuation subassembly fully to the second position.

In accordance with an example embodiment of the present disclosure a nut assembly for use with a leadscrew may comprise a nut body including a base and a first arm extending from an end of the base. The base may include a bore extending therethrough along an axis of the nut body. The first arm may include a threaded region spaced from the base on a face of the arm most proximate the axis. The first arm may also include a first projection. The nut assembly may further comprise an actuation subassembly including a first body disposed in flanking relation to the first arm. The first body may include a first track into which the first projection extends. The nut assembly may further comprise an actuator coupled to the actuation subassembly and displaceable to translationally drive the actuation subassembly from a first position to a second position. The first track may be angled such that the first arm is pivoted, via interaction of the first projection with the first track, between a position proximate the axis when the actuation subassembly is in the first position and a position more distal the axis when the actuation subassembly is in the second position. The nut assembly may further comprise at least one bias member configured to urge the actuation subassembly to the first position.

In some embodiments, the first arm may include a first slot extending therethrough. In some embodiments, the nut assembly may include a second arm extending from the end of the base. The second arm may extend from the end of the base in a position opposite the first arm and including a threaded region spaced from the base. In some embodiments, the second arm may include a second projection which extends into a second track defined in the first body. In some embodiments, the second track may be angled such that the second arm is pivoted, via interaction of the second projection with the second track, between a position proximate the axis when the actuation subassembly is in the first position and a position more distal the axis when the actuation subassembly is in the second position. In some embodiments, the second arm may include a second slot extending therethrough. In some embodiments, the first arm may include a second projection and the actuation subassembly may further include a second body disposed in flanking relation to the first arm. The first arm may be intermediate the first and second body. The second body may include a second track into which the second projection extends. In some embodiments, the second track may be angled such that the first arm is pivoted, via interaction of the second projection with the second track, between the position proximate the axis when the actuation subassembly is in the first position and the position more distal the axis when the actuation subassembly is in the second position. In some embodiments, the actuator may include at least one wire coupled to the actuation subassembly. In some embodiments, the bore may be configured to accept a leadscrew extending through the nut assembly along the axis. In some embodiments, the base may include a flange and the first body may include a pocket. One of the at least one bias member may be seated in the pocket and in contact with the flange. In some embodiments, the nut assembly may further comprise a housing surrounding the nut body and the actuation subassembly.

In accordance with an example embodiment of the present disclosure an exemplary method of adjusting the position of a drive head of a syringe pump relative to a housing of the syringe pump may comprise displacing a nut actuator of a syringe pump from a first position to a second position. The method may further comprise driving, via displacement of the nut actuator, an actuation portion of a nut assembly from a resting position to an actuated position. The actuator portion may include a first angled track. The method may further comprise deflecting a threaded region of a first arm extending from a base body of the nut assembly out of engagement with a leadscrew of the syringe pump as the first angled track is displaced relative to a first pin projecting from the first arm when the actuation portion is displaced from the resting position to the actuated position. The method may further comprise moving the drive head to a desired position relative to the housing.

In some embodiments, the method may further comprise returning the actuation portion from the actuated position to the resting position via displacement of the nut actuator from the second position to the first position. In some embodiments, the method may further comprise returning the first arm into engagement with the leadscrew as the first angled track is displaced relative to the pin when the actuation portion is displaced from the actuated position to the resting position. In some embodiments, the method may further comprise biasing, with at least one bias member, the actuation portion towards the resting position. In some embodiments, the actuation portion may include a second angled track and the method may further comprise deflecting a threaded region of a second arm extending from the base body out of engagement with the leadscrew as the second angled track is displaced relative to a second pin projecting from the second arm when the actuation portion is displaced from the resting position to the actuated position. In some embodiments, the method may further comprise returning the actuation portion from the actuated position to the resting position via displacement of the nut actuator from the second position to the first position and returning the second arm into engagement with the leadscrew as the second angled track is displaced related to the second pin while the actuation portion is returned to the resting position. In some embodiments, the method may further comprise re-engaging the threaded region of the first arm with the leadscrew. In some embodiments, displacing the nut actuator may comprise displacing a wire coupled to the actuation portion of the nut assembly.

In accordance with another example embodiment of the present disclosure an example drive assembly for a medical pump may comprise a leadscrew extending along an axis. The drive assembly may further comprise a motor operatively coupled to the leadscrew and configured to generate rotational displacement of the motor. The method may further comprise a nut assembly. The nut assembly may comprise a nut body including a base having a cantilevered first arm. The base may include a bore through which the leadscrew extends. The first arm may include a first projection and a first threaded region. The nut assembly may further comprise an actuation subassembly including a first body disposed in flanking relation to the first arm. The first body may include a first track into which the first projection extends. The nut assembly may further comprise an actuator coupled to the actuation subassembly and displaceable to translationally drive the actuation subassembly relative to the nut body from a first position to a second position. The nut assembly may further comprise at least one bias member configured to urge the actuation subassembly toward the first position. When the actuation sub assembly is in the second position, the first track may hold the first threaded region, via the first projection, in a position distal to the leadscrew and when the actuation subassembly is displaced toward the first position with respect to the nut body. The path of the first track may drive the first threaded region, via the first projection, into engagement with the leadscrew.

In some embodiments, the first arm may include a first slot extending therethrough. In some embodiments, the base may include a cantilevered second arm disposed in a position opposite the first arm. The second arm may include a second threaded region. In some embodiments, the second arm may include a second projection which extends into a second track defined in the body. In some embodiments, the second track may hold the second threaded region, via the second projection, in a position distal to the leadscrew and when the actuation subassembly is displaced toward the first position with respect to the nut body, the path of the second track may drive the second threaded region, via the second projection, into engagement with the leadscrew. In some embodiments, the second arm may include a second slot extending therethrough. In some embodiments, the first arm may include a second projection and the actuation subassembly may further include a second body disposed in flanking relation to the first arm. The first arm may be intermediate the first and second body. The second body may include a second track into which the second projection extends. In some embodiments, the second track may hold the first threaded region, via the second projection, in a position distal to the leadscrew and when the actuation subassembly is displaced toward the first position with respect to the nut body. The path of the second track may drive the first threaded region, via the second projection, into engagement with the leadscrew. In some embodiments, the actuator may include at least one wire coupled to the actuation subassembly. In some embodiments, the bore may be dimensioned such that the leadscrew does not contact walls of the bore. In some embodiments, the base may include a flange and the first body may include a pocket. One of the at least one bias member being seated in the pocket and in contact with the flange. In some embodiments, the nut assembly may further comprise a housing surrounding the nut body and the actuation subassembly.

In accordance with another example embodiment of the present disclosure an example nut assembly for use with a leadscrew may comprise a stationary portion. The nut assembly may further comprise a first arm coupled to the stationary portion and pivotable relative to the stationary portion. The first arm may include a threaded region on a face of the arm most proximate the axis. The nut assembly may further comprise an actuation subassembly including a body having an aperture extending therethrough. The aperture may define at least one guide surface. The nut assembly may further comprise an actuator coupled to the actuation subassembly and displaceable to translationally drive the actuation subassembly from a first position to a second position. One of the at least one guide surface may be aligned with the first arm and configured to collide with the first arm and pivotally displace the first arm from a position distal an axis of the nut assembly to a position more proximal the axis when the actuation subassembly is displaced toward the second position. The nut assembly may further comprise at least one bias member configured to urge the actuation subassembly to the first position.

In some embodiments, the nut assembly may further comprise a return spring coupled to the first arm. The return spring may be configured to urge the first arm to the position distal the axis when the actuation subassembly is displaced toward the first position. In some embodiments, the guide surface may be a tapered portion of the sidewall of the aperture. In some embodiments, the tapered portion may include a first portion and a second portion. The first portion may be more sharply tapered than the second portion. In some embodiments, the stationary portion may be a housing which at least partially surrounds the actuation subassembly and first arm. In some embodiments, the first arm may be coupled to the stationary portion by a pivot pin extending through the first arm and having a pivot pin axis. The first arm may be pivotable about the pivot pin axis. In some embodiments, the nut assembly may include a second arm coupled to the stationary portion. The second arm may include a threaded region and being disposed in opposition to the first arm. In some embodiments, the at least one guide surface may include a first guide surface and a second guide surface disposed in opposition to one another about the aperture. In some embodiments, the second guide surface may be aligned with the second arm and configured to collide with the second arm and pivotally displace the second arm from a position distal the axis of the nut assembly to a position more proximal the axis when the actuation subassembly is displaced toward the second position. In some embodiments, the first and second arm may be coupled to one another via a return spring configured to urge the first and second arms to pivot toward a position in which the threaded region of the first arm and the threaded region of the second arm are maximally separated from one another. In some embodiments, the return spring may be a torsion spring.

In accordance with another example embodiment of the present disclosure an exemplary method of adjusting the position of a drive head of a syringe pump relative to a housing of the syringe pump may comprise displacing a nut actuator of a syringe pump from a first position to a second position. The method may further comprise driving an actuation portion of a nut assembly coupled to the nut actuator from an engagement position to a disengagement position. The actuation portion may comprise a first ramped face which contacts a tip of the first arm coupled to a base portion of the nut assembly when the actuation portion is in the engagement position and is displaced in a direction away from the tip of the first arm as the actuation portion is driven from the engagement position to the disengagement position. The method may further comprise engendering pivoting of the first arm, via urging of at least one bias member, as the first ramped face is displaced in the direction away from the tip of the first arm such that a threaded region of the first arm is displaced out of engagement with a leadscrew of the syringe pump. The method may further comprise moving the drive head to a desired position relative to the housing.

In some embodiments, the method may further comprise biasing, with at least one bias member, the actuation portion to the disengagement position. In some embodiments, the actuation portion may include a second ramped face which contacts a tip of a second arm coupled to the base portion when the actuation portion is in the engagement position and driving the actuation portion from the engagement position to the disengagement position may comprise displacing the second ramped face in a direction away from the tip of the second arm. In some embodiments, the method may further comprise engendering pivoting of the second arm, via urging of the at least one bias member, as the second ramped face is displaced in the direction away from the tip of the second arm such that a threaded region of the second arm is displaced out of engagement with the leadscrew. In some embodiments, the method may further comprise reengaging the threaded region of the first arm with the leadscrew. In some embodiments, the method may further comprise returning the actuation portion from the disengagement position to the engagement position via displacement of the nut actuator from the second position to the first position. In some embodiments, the method may further comprise driving the first ramped face against the tip of the first arm and engendering pivoting of the first arm such that the threaded region of the first arm is pressed into engagement with the leadscrew.

In accordance with another example embodiment of the present disclosure a target assembly for an inductive position sensor may comprise a main body having a back bone including a first arm extending from a first end region of the backbone and a second arm extending from an opposing end region of the back bone. The main body may further comprise a central peg extending from the back bone. The central peg may comprise a beveled tip region opposite the back bone. The target assembly may further comprise at least one coil. One of the at least one coil may be wound around the central peg. The target assembly may further comprise a capacitor parallel to one of the at least one coil.

In some embodiments, the main body may be constructed of a ferrite material. In some embodiments, the at least one coil may include a pair of arm coils respectively wound around the first arm and second arm. In some embodiments, the arm coils may be 180° out of phase with the coil would around the central peg. In some embodiments, the beveled tip has a width of less than 0.5 mm at the end of the beveled tip most distal to the back bone. In some embodiments, the first arm, second arm, and third arm each extend in the same direction. The length of the central peg may be less than the lengths of each of the first arm and second arm. In some embodiments, the coil wound around the central peg may be positioned adjacent the back bone and spaced from the beveled tip region. In some embodiments, the beveled tip may be formed by a double bevel.

In accordance with an example embodiment of the present disclosure an example linear position sensing assembly may comprise a printed circuit board. The printed circuit board may comprise a plurality of sensing traces including short and long period sinusoidal sensing traces, and short and long period cosinusoidal sensing traces. Each of the plurality of sensing traces may be doubled back at least once across a trace originating layer and 180° out of phase across a return layer of the printed circuit board that are dedicated to each sensing trace. The sensing assembly may further comprise an excitation coil surrounding the sensing traces and having flanking regions extending along either side of the plurality of sensing traces. The sensing assembly may further comprise a set of ferrite bodies coupled to a side of the PCB. A first of the ferrite bodies may be aligned with the plurality of sensing traces. A second and third of the ferrite bodies may each be aligned with one of the flanking regions. The sensing assembly may further comprise a target assembly configured to displace in a range over the sensing traces. The target assembly may include a coil and a capacitor configured to form a resonant circuit. The target assembly may have a main body with a peg extending toward the sensing traces. A tip region of the peg may be beveled to a thinnest width which is a fraction of the shortest period length of any of the plurality of sensing traces.

In some embodiments, each of the short period sinusoidal and cosinusoidal sensing traces may be doubled back at least four times. In some embodiments, at least the short period sinusoidal and cosinusoidal sensing traces may be doubled back at least eight times. In some embodiments, the short period sinusoidal and cosinusoidal sensing traces may have periods of 0.1 inch or less. In some embodiments, the short period sinusoidal and cosinusoidal sensing traces may have periods of 2 mm or less. In some embodiments, the long period sinusoidal and cosinusoidal sensing traces may have a period at least equivalent to the displacement range of the target assembly. In some embodiments, the excitation coil may be disposed on an exterior layer of the PCB shared with a portion of one of the sensing traces. In some embodiments, the main body of the target assembly may be constructed of a ferrite material. In some embodiments, the peg may extend from a central region of a back bone of the main body and the main body may include a pair of arms on opposing sides of the peg. Each arm may extend over a respective flanking region of the excitation coil. In some embodiments, the coil may be positioned on the peg and the target assembly may include a pair of arm coils. Each of the pair of arm coils may be disposed around a respective arm of the pair of arms. In some embodiments, the tip region of the peg may be beveled to a thinnest width no more than 10% of the shortest period length of any of the plurality of sensing traces.

In accordance with another example embodiment of the present disclosure an example syringe pump may comprise a drive assembly including a drive motor, a leadscrew and a nut. The drive motor may be configured to engender displacement of the nut along the leadscrew when powered. The syringe pump may further comprise a drive head coupled to the drive assembly and configured to displace as the nut is displaced along the leadscrew. The syringe pump may further comprise a linear position sensing assembly. The linear position sensing assembly may comprise a circuit board including an excitation coil. The linear position sensing assembly may further comprise a set of sensing traces each doubled back at least once in antiphase on different layers of the circuit board. The sensing trace may including short period sinusoidal and cosinusoidal sensing traces and long period sinusoidal and cosinusoidal sensing traces. The linear position sensing assembly may further comprise a resonant target assembly coupled with the drive assembly and configured to displace in tandem with the drive head. The target assembly may include a beveled projection positioned over the sensing traces. The syringe pump may further comprise a controller configured to power the excitation coil to resonate the target assembly. The controller further configured to monitor an induced voltage on each of the sensing traces and determine a position of the target assembly based at least in part on the induced voltage on each sensing trace.

In some embodiments, the target assembly may be constructed of a ferrite material and includes two arms disposed over portions of the excitation coil. In some embodiments, the target assembly may be coupled to a carriage which displaces in tandem with the drive head. In some embodiments, the carriage may displace along at least one bearing surface on a rigid back bone of the syringe pump and the drive head may also be configured to displace along at least one drive head bearing surface on the rigid back bone. In some embodiments, the controller may be further configured to govern displacement of the drive head of the syringe pump based at least in part upon the position of the target assembly. In some embodiments, each of the short period sinusoidal and cosinusiodal sensing traces may be doubled back at least four times. In some embodiments, each of the short period sinusoidal and cosinusoidal sensing traces may have a period of no more than 2 mm. In some embodiments, the beveled projection may include a tip region which has a thinnest width no more than 30% of the shortest period length of any of the plurality of sensing traces. In some embodiments, the circuit board may include a ferrite body disposed in alignment with the sensing traces. In some embodiments, the excitation coil may include regions extending adjacent the sensing traces. The regions each being associated with ferrite bodies. In some embodiments, the controller may be configured to determine a position of the target assembly with an output resolution of less than one micron. In some embodiments, the magnetic field generated as the target resonates may have a thinnest region no more than 15% the shortest period length of any of the plurality of sensing traces.

In accordance with another example embodiment of the present disclosure an exemplary method of determining the position of the drive head of a syringe pump may comprise illuminating an excitation coil of a circuit board. The method may further comprise resonating a resonant circuit in a target assembly having a projection with a beveled end. The method may further comprise receiving, with a controller, a reading of a voltage induced on each of a plurality of sensing traces positioned adjacent the beveled end and on the circuit board. The plurality of sensing traces may include short period sinusoidal and cosinusoidal sensing traces and long period sinusoidal and cosinusoidal sensing traces. Each sensing trace may be doubled back at least once in antiphase on different layers of the circuit board. The method may further comprise analyzing, with a controller, the induced voltage on each of the sensing traces to determine a position of the target assembly in relation sensing traces.

In some embodiments, analyzing the induced voltage on each of the sensing traces may comprise checking the induced voltages against a look up table. In some embodiments, the method may further comprises, halting illuminating the excitation coil before receiving the reading of a voltage induced on each of the plurality of sensing traces. In some embodiments, analyzing the induced voltage on each of the sensing traces may comprise analyzing the induced voltage from each of the long period traces to determine a particular sinusoidal period of the short period sinusoidal trace and a particular consinusoidal period of the short period cosinusoidal trace over which the beveled end is positioned. In some embodiments, analyzing the induced voltage on each of the sensing traces may further comprise analyzing the induced voltage from each of the short period sinusoidal and consinusoidal trace and determining the position of the target assembly. In some embodiments, determining the position of the target assembly may comprise determining the position of the target assembly with submicron resolution. In some embodiments, the method may further comprise generating a command for a drive motor of the syringe pump based at least in part upon the determined position of the target assembly. In some embodiments, the method may further comprise providing a ferrite body aligned with the sensing traces on the circuit board and associating additional ferrite bodies with portions of the excitation coil which are adjacent the sensing traces.

### BRIEF DESCRIPTION OF THE DRAWINGS

These and other aspects will become more apparent from the following detailed description of the various embodiments of the present disclosure with reference to the drawings wherein

FIG. 1 depicts a perspective view of an exemplary syringe pump;

FIG. 2 depicts a perspective view of an exemplary syringe pump with a small volume syringe;

FIG. 3 depicts a perspective view of an exemplary syringe pump with a large volume syringe;

FIG. 4 depicts a perspective view of a syringe in place in an exemplary syringe retention assembly;

FIG. 5 depicts an example syringe retention assembly;

FIGS. 6A-B depict plan views of an example syringe retention assembly;

FIG. 7 depicts a perspective view of a portion of an example syringe pump with an example barrel grasper assembly;

FIG. 8 depicts a perspective view of a syringe in place in an example barrel grasper assembly;

FIG. 9 depicts a cross-sectional view of a syringe in place within an example barrel grasper assembly;

FIG. 10 depicts a perspective view of a portion of an example syringe pump and a syringe in place in an example barrel grasper assembly of the syringe pump;

FIG. 11 depicts a top plan view of an example barrel grasper assembly;

FIG. 12 depicts a bottom plan view of an exemplary barrel grasper assembly;

FIG. 13 depicts a cross-sectional view of an example barrel grasper assembly;

FIG. 14 depicts a perspective view of a syringe in place in an example barrel grasper assembly;

FIG. 15 depicts a plan view of an example barrel retaining body which may be included in example barrel grasping assemblies;

FIGS. 16A-B depict breakaway perspective views of an exemplary syringe retention assembly which may be included in syringe pumps such as the example syringe pumps described herein;

FIGS. 17A-B depict breakaway perspective views of the example syringe retention assembly of FIG. 16A with an example barrel flange grasper assembly in an open state and an example barrel clamp assembly in an open and swung out state;

FIG. 18 depicts a perspective view of an example barrel clamp assembly;

FIGS. 19A-B depict breakaway perspective views of the example syringe retention assembly of FIG. 17A with a syringe in place in a trough defined in the exterior surface of the syringe retention assembly;

FIGS. 20A-B depict breakaway perspective views of the example syringe retention assembly of FIG. 17A with an example barrel flange grasper assembly in a retaining state against a barrel flange of a syringe;

FIGS. 21A-B depict breakaway perspective views of the example syringe retention assembly of FIG. 17A with an example barrel clamp assembly in a retaining state with respect to a syringe;

FIG. 22A depicts a perspective view of an example drive head with a plurality of exemplary plunger flange grasper bodies;

FIG. 22B depicts the example drive head of FIG. 22A with the plunger flange grasper bodies in the open position;

FIG. 23 depicts a perspective view of the example drive head of FIG. 22A with a portion of the housing removed;

FIG. 24 depicts a perspective view of an example actuator, example gearing, and example plunger flange grasper bodies which may be included in example drive heads for a syringe pump;

FIG. 25 depicts a perspective view of an example drive head with a set of plunger flange grasper bodies;

FIG. 26A depicts a perspective view of certain components of the example drive head of FIG. 25;

FIG. 26B depicts a perspective view of certain components of the example drive head of FIG. 25;

FIG. 27 depicts an exploded view of a certain components which may be included in example drive head for a syringe pump;

FIG. 28A depicts a detailed view of a flange of a plunger of a syringe being contacted by exemplary plunger flange grasper bodies having beveled snugging faces;

FIG. 28B depicts a detailed view of a flange of a plunger of a syringe being held against a contact for a load cell arrangement by exemplary plunger flange grasper bodies having beveled snugging faces;

FIG. 29 depicts a partially exploded view of an example sensing assembly with a motor driven load cell;

FIG. 30 depicts a top plan view of a portion of an example drive head including the example sensing assembly of FIG. 29;

FIG. 31A depicts a cross-sectional view taken at the indicated cut plane of FIG. 30;

FIG. 31B depicts a cross-sectional view taken at the indicated cut plane of FIG. 30;

FIG. 32 depicts a perspective view of a portion of an example drive head including the exemplary sensing assembly of FIG. 30;

FIGS. 33A-B depict block diagrams of an example drive head including an example force concentrator;

FIG. 34 depicts a plan view of a portion of an example drive head including an exemplary force concentrator;

FIG. 35 depicts a perspective view of an example force concentrator;

FIG. 36 depicts a block diagram of a portion of an example drive head including an example floating assembly;

FIG. 37 depicts a block diagram of a portion of an example drive head including an example floating assembly;

FIGS. 38A-39B depicts a number of block diagram views of an example drive assembly which may be used in a syringe pump such as any of those described herein;

FIG. 40 depicts a perspective view of an example drive assembly including a belt drive;

FIG. 41A depicts an exploded view of an example clutch assembly;

FIG. 41B depicts a cross-sectional view of an example clutch assembly;

FIG. 42A depicts an exploded view of an example clutch assembly;

FIG. 42B depicts a perspective cross-sectional view of an example clutch assembly;

FIG. 43A depicts a view of an example clutch assembly and leadscrew;

FIG. 43B depicts a cross-sectional view of an example clutch assembly and leadscrew;

FIG. 44A depicts a view of a portion of an example drive assembly which may be used in a syringe pump such as any of those described herein;

FIG. 44B depicts a cross-sectional view of FIG. 44A;

FIG. 45 depicts a perspective view of a portion of an example drive assembly which may be used in a syringe pump such as any of those described herein;

FIG. 46 depicts a detailed view of the indicated region of FIG. 45;

FIG. 47A depicts a perspective view of an example nut tube;

FIG. 47B depicts a detailed view of the indicated region of FIG. 47A;

FIG. 48A depicts a perspective view of an example nut tube and an example nut;

FIG. 48B depicts a cross-sectional view of the example nut tube and example nut depicted in FIG. 48A;

FIG. 49A depicts an exploded view of an example nut and a portion of an example nut tube;

FIG. 49B depicts a cross-sectional view of the example nut and example nut tube depicted in FIG. 49A;

FIG. 50 depicts an exploded view of part of an example driving portion of a drive assembly which may be included in a syringe pump such as any of the example syringe pumps described herein;

FIG. 51 depicts an exploded view of part of an example driving portion of a drive assembly which may be included in a syringe pump such as any of the example syringe pumps described herein;

FIG. 52 depicts an exploded view of part of an example driving portion of a drive assembly which may be included in a syringe pump such as any of the example syringe pumps described herein;

FIG. 53 depicts an exploded view of part of an example driving portion of a drive assembly which may be included in a syringe pump such as any of the example syringe pumps described herein;

FIG. 54 depicts an exploded view of part of an example driving portion of a drive assembly which may be included in a syringe pump such as any of the example syringe pumps described herein;

FIG. 55 depicts an exploded view of part of an example driving portion of a drive assembly which may be included in a syringe pump such as any of the example syringe pumps described herein;

FIG. 56 depicts an exploded view of part of an example driving portion of a drive assembly which may be included in a syringe pump such as any of the example syringe pumps described herein;

FIG. 57A depicts a cross-sectional view of part of an example driving portion of a drive assembly which may be included in a syringe pump such as any of the example syringe pumps described herein;

FIG. 57B depicts an exploded view of part of an example driving portion of a drive assembly which may be included in a syringe pump such as any of the example syringe pumps described herein;

FIG. 58A depicts a perspective view of an example driving portion of a drive assembly which may be included in a syringe pump such as any of the example syringe pumps described herein;

FIG. 58B depicts a plan view of an example driving portion of a drive assembly which may be included in a syringe pump such as any of the example syringe pumps described herein;

FIG. 58C depicts a cross-sectional view of an example driving portion of a drive assembly which may be included in a syringe pump such as any of the example syringe pumps described herein;

FIG. 59A depicts a cross-sectional view of an example nut assembly which may be actuated into and out of engagement with a leadscrew;

FIG. 59B depicts a side view of the example nut assembly of FIG. 59A;

FIG. 59C depicts another cross-sectional view of the example nut assembly of FIG. 59A;

FIG. 59D depicts a cutaway view of the example nut assembly of FIG. 59A in place within a portion of an example drive assembly;

FIG. 59E depicts an exploded view of the example nut assembly of FIG. 59A;

FIG. 60A depicts a perspective view of another example nut assembly which may be actuated into and out of engagement with a leadscrew;

FIG. 60B depicts a cross-sectional view of the example nut assembly of FIG. 60A;

FIG. 60C depicts another cross-sectional view of the example nut assembly of FIG. 60A;

FIG. 60D depicts an exploded view of the example nut assembly of FIG. 60A;

FIG. 61 depicts a plan view of a portion of an example drive head which may be included in a syringe pump such as any of the example syringe pumps described herein;

FIG. 62A depicts a plan view of a portion of an example drive head which may be included in a syringe pump such as any of the example syringe pumps described herein;

FIG. 62B depicts a plan view of a portion of an example drive head which may be included in a syringe pump such as any of the example syringe pumps described herein;

FIG. 62C depicts a detailed view of a portion of an example drive head which may be included in a syringe pump such as any of the example syringe pumps described herein;

FIG. 63A depicts a detailed view of a portion of an example drive head which may be included in a syringe pump such as any of the example syringe pumps described herein;

FIG. 63B depicts a detailed view of a portion of an example drive head which may be included in a syringe pump such as any of the example syringe pumps described herein;

FIG. 64A depicts a cross-sectional view of an example drive assembly with a guide assembly which may be used in a syringe pump such as any of those described herein;

FIGS. 64B-C depict a cross-sectional views of FIG. 64A;

FIG. 65A depicts a plan view of a first side of an example linear position sensing assembly;

FIG. 65B depicts a plan view of a second side of the example linear position sensing assembly of FIG. 65A;

FIG. 66A depicts an example target assembly which may be included in example linear position sensing assemblies such as those described herein;

FIG. 66B depicts an schematic view of the example target assembly of FIG. 66A;

FIG. 67A depicts an example target assembly which may be included in example linear position sensing assemblies such as those described herein;

FIG. 67B depicts a schematic view of the example target assembly of FIG. 67A;

FIG. 68 depicts a simulated extruded 2D field strength coupling map for a portion of an example linear position sensing assembly with the target assembly of FIG. 67A;

FIGS. 69-70 respectively depict plots of simulated induced voltages in exemplary short and long period sensing traces generated with displacement of a target assembly over the traces;

FIGS. 71-72 respectively depict plots of the output of a function into which simulated induced voltages in example short and long period sensing traces may be fed;

FIGS. 73A-H depict example short and long period traces which may be included on layers of a printed circuit board of an exemplary linear position sensing assembly;

FIG. 74 depicts a perspective view of an example carriage with a target assembly coupled thereto which may be included in an example drive assembly for a syringe pump;

FIG. 75 depicts a plan view of the example carriage of FIG. 74;

FIG. 76 depicts a perspective view of an example carriage and rigid body which may be included in an example syringe pump; and

FIG. 77 depicts a cross-sectional view of an example carriage and an example linear position sensing assembly centered within a rigid support structure of by an example mounting body.

### DETAILED DESCRIPTION

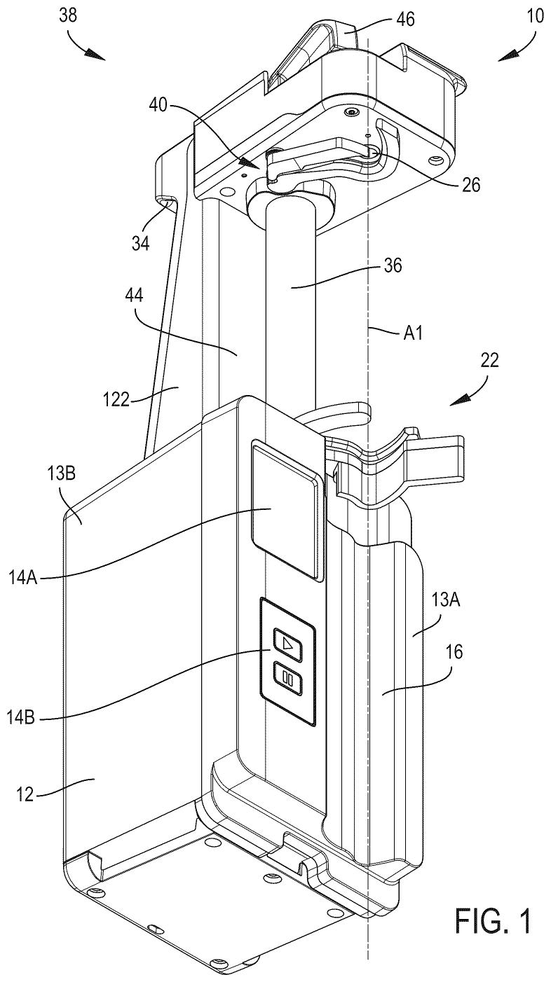

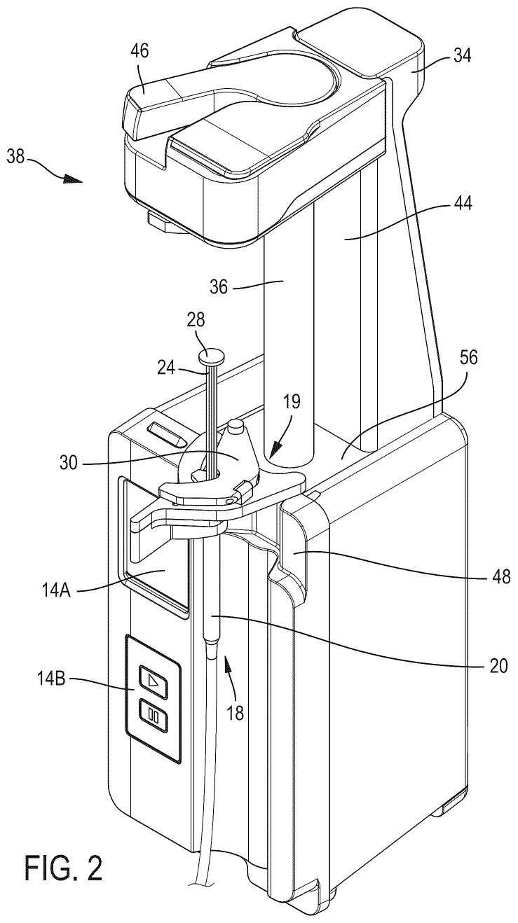

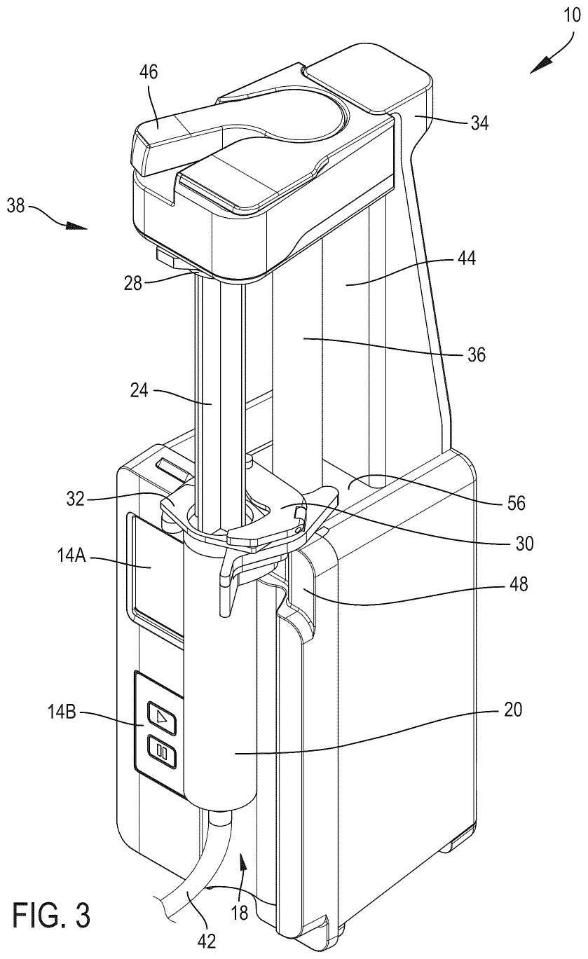

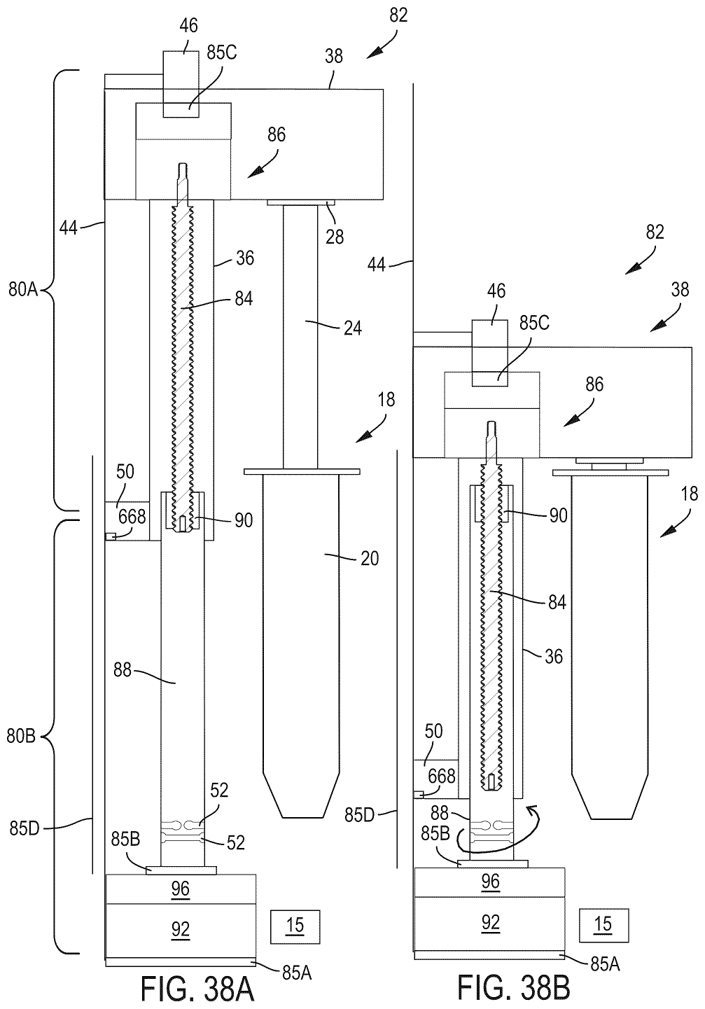

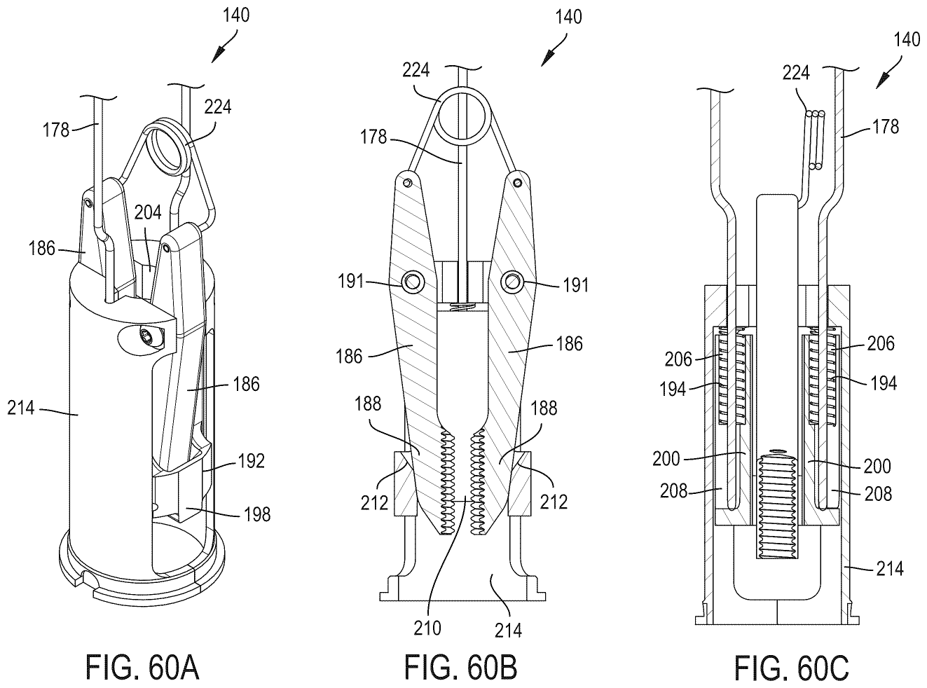

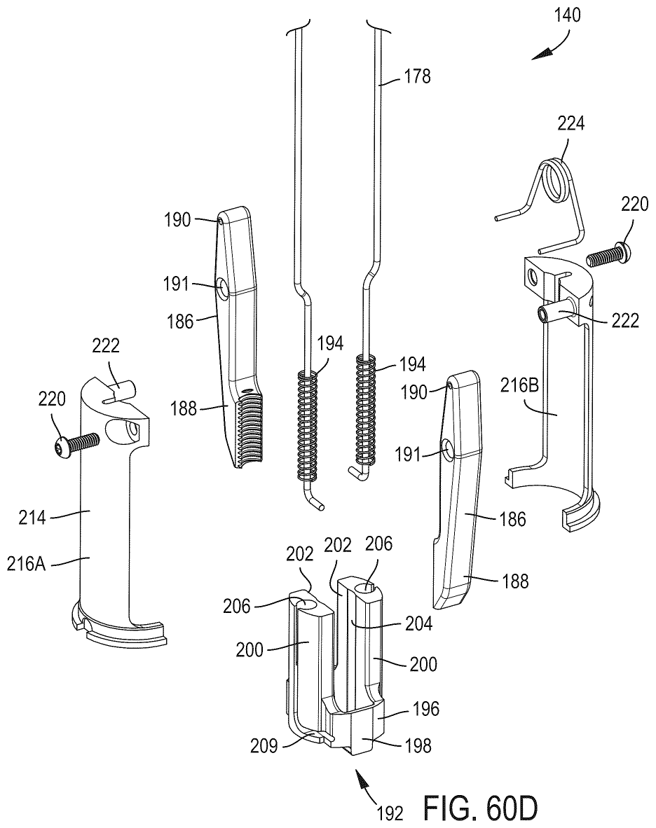

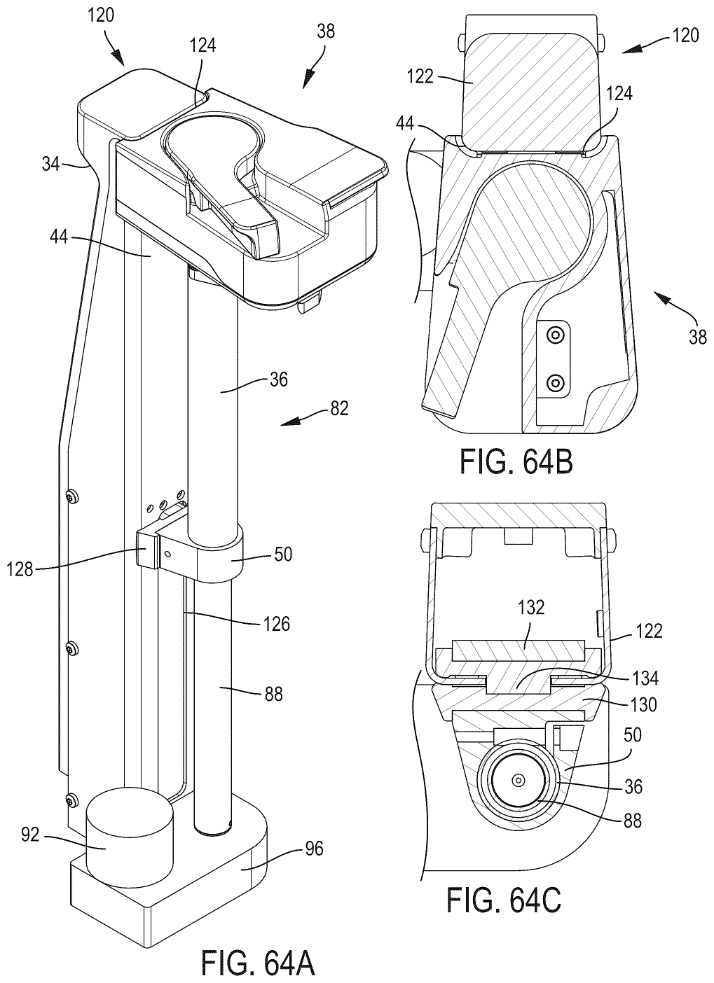

Referring now to FIGS. 1-3, perspective views of an exemplary syringe pump 10 are depicted. The example syringe pump 10 includes a main housing 12. The main housing 12 may include a front housing or bezel portion 13A and a rear housing or rear enclosure 13B which may be coupled to one another. The main housing 12 may enclose portions of a drive assembly 82 (see, e.g., FIGS. 38A-40, FIGS. 44A-49B, FIGS. 59A-E, FIGS. 60A-D, FIGS. 64A-C) various electronics, sensors, communication hardware, a controller 15 (see, e.g., FIG. 38A) including one or more processors, etc. The main housing 12 may include one or more user interface 14A, 14B. For example, the syringe pump 10 may include a graphical user interface 14A and a keypad interface 14B which may be accessible on the main housing 12. The main housing 12 may also include a housing trough 16 recessed into a portion (e.g. the front bezel 13A) of the main housing 12. The housing trough 16 may accommodate a portion of the barrel 20 of various syringes 18 (see, e.g., FIGS. 2-3) when syringes 18 are coupled to the syringe pump 10. The user interface(s) 14A, B may be included on the same face of the main housing 12 as the housing trough 16.

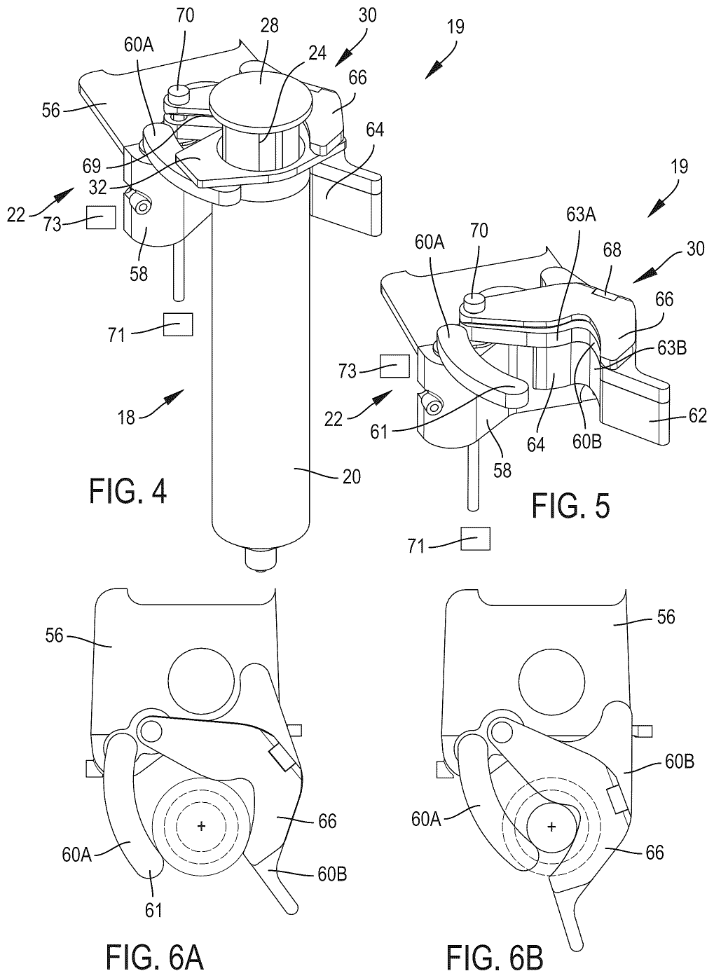

A syringe retention assembly 19 may be coupled to the main housing 12. A syringe retention assembly 19 may hold a syringe 18 in place against a syringe pump 10 and may, in certain examples, locate the syringe 18 in a desired positon with relation to the syringe pump 10. Certain example syringe retention assemblies 19 may include a barrel grasper assembly 22. Any of the barrel grasper assemblies 22 shown and described in relation to FIGS. 4-21B may, for instance be used. Barrel grasper assemblies 22 may be transitioned between an opened state and a syringe retaining state to couple a syringe 18 in place on the main housing 12 of the syringe pump 10. The housing trough 16 may include a recess 48 adjacent the barrel grasper assembly 22 to provide clearance for portions of the barrel grasper assembly 22 as the barrel grasper assembly 22 is operated. Barrel grasper assemblies 22 may be arranged to accept any of a variety of a set of accepted syringes 18 which may have a range of volumes. In certain examples, barrel grasper assemblies 22 may retain any syringe 18 having a volume from 1 cc or less up to and including 60 cc or greater. Displacement of portions of the barrel grasper assembly 22 may be tracked with one or more sensor (e.g. magnetic sensor, rotary potentiometer). Thus, when in a syringe retaining state, the position of portions of the barrel grasper assembly 22 may be known. This may allow for a controller 15 of the syringe pump 10 to determine the diameter of the barrel 20 of a syringe 18 in order to identify the syringe 18 installed on the syringe pump 10. This position data may also assist in verification, by a controller 15, that a syringe 18 is present and properly coupled to the syringe pump 10.

As will be further described in greater detail elsewhere in the specification, various barrel grasper assemblies 22 may locate retained syringes 18 such that the center axis of the bore of the barrel 20 of the syringe 18 is in alignment (e.g. coaxial) with a fiducial reference axis A1. In certain examples, the reference axis A1 may be an axis that runs through an interface between an installed syringe plunger 24 and a load cell 330 (see, e.g., FIG. 32) or sensor which may output data indicative of pressure in an installed syringe 18. The interface may, for example be a nub or button 26 which contacts a flange 28 or thumb press disk of the plunger 24 of the syringe 18. In other embodiments, the barrel grasper assembly 22 of a syringe pump 10 may retain the syringe 18 against the housing trough 16 without centering the axis of the barrel 20 bore. In such examples, the position of the axis of the barrel 20 bore may differ depending on the size of the syringe 18.

A barrel flange grasper assembly 30 may also be included as part of the syringe retention assembly 19 in certain embodiments (see, e.g., FIGS. 4-6B). The barrel flange grasper assembly 30 may be attached to or a part of the barrel grasper assembly 22 in certain examples. The barrel flange grasper assembly 30 may retain a barrel flange 32 (see, e.g., FIG. 3) of an installed syringe 18 in place with respect to the syringe pump 10. In certain examples, the barrel flange grasper assembly 30 may capture the barrel flange 32 against a portion of the barrel grasper assembly 22. Any of the barrel flange grasper assemblies 30 shown and described herein may be used. The position of at least a portion of the barrel flange grasper assembly 30 may be tracked by one or more sensors. The one or more sensor may output a signal indicative of a physical characteristic of the barrel flange 32 of an installed syringe 18 (e.g. thickness). This data may be used to assist in identifying the type of syringe 18 installed on the syringe pump 10. This data may also be used to ensure that a syringe 18 is present and/or properly retained on the syringe pump 10.

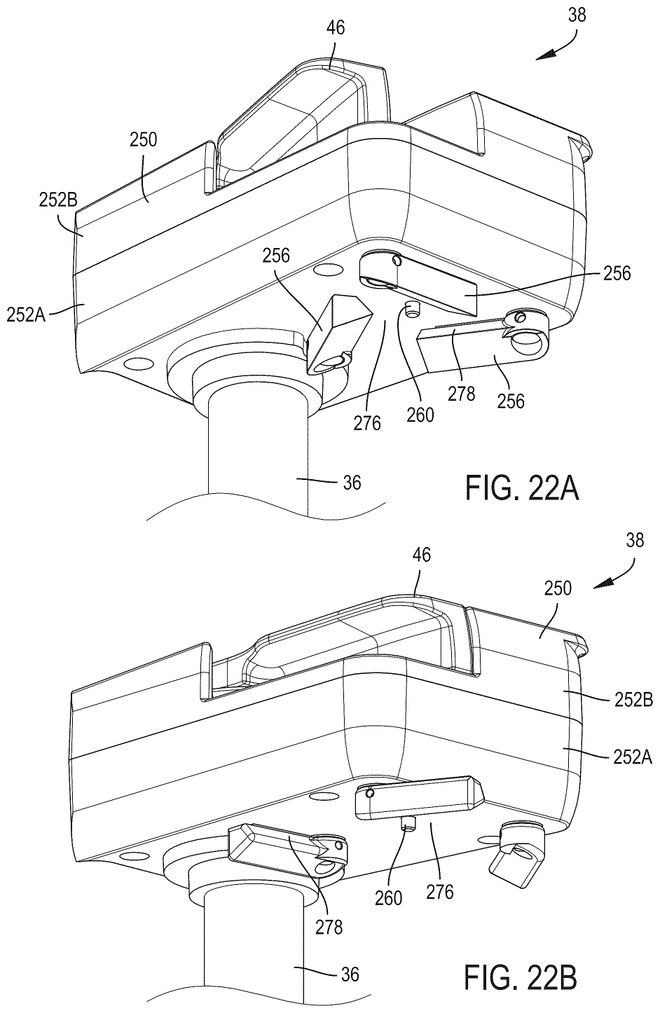

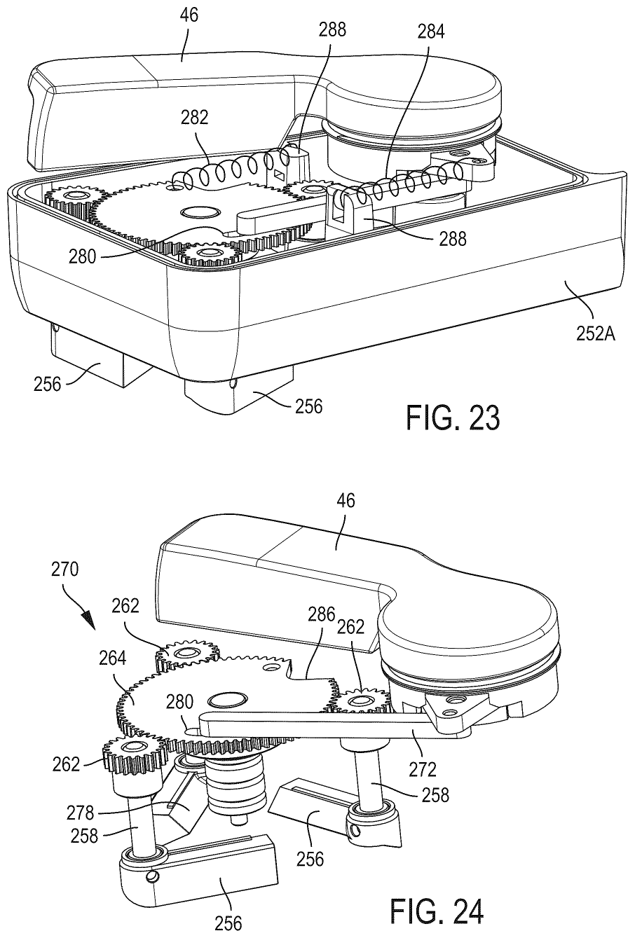

A drive head tube 36 may extend from the main housing 12 and may be coupled to a drive head 38. The drive head 38 may be coupled to a terminal end of the drive head tube 36 distal to the main housing 12. The drive head 38 may include a plunger flange grasper assembly 40. The plunger flange grasper assembly 40 may be any plunger flange grasper assemblies 40 described herein (e.g. such as those shown and described in relation to FIGS. 22A-28B, FIGS. 34-37). A plunger flange grasper assembly 40 may retain the plunger flange 28 of a syringe 18 in place with respect to the drive head 38. Plunger flange grasper assemblies 40 may also hold the plunger flange 28 snuggly against the drive head 38 of the syringe pump 10. As will be further described elsewhere in the specification, various plunger flange grasper assemblies 40 may locate retained plunger flanges 28 such that the center of the plunger flange 28 is aligned with a fiducial reference axis A1. This reference axis may be the same reference axis that the barrel grasper assembly 22 centers the bore of the syringe 18 barrel 20 upon. As the drive assembly 82 (see, e.g., FIGS. 38A-40, FIGS. 44A-49B, FIGS. 59A-E, FIGS. 60A-D, FIGS. 64A-C of the syringe pump 10 is operated (e.g. governed by a controller 15 in the main housing 12), the drive head tube 36 and drive head 38 may be displaced translationally with respect to the main housing 12 of the syringe pump 10. As the drive head 38 is displaced toward the main housing 12, the plunger 24 may be driven into the barrel 20 of the syringe 18 and fluid contained within the syringe 18 may be urged out of the syringe 18 and into an attached fluid administration line 42 (see, e.g., FIG. 3) in fluid communication (e.g. via intravenous needle) with a patient (e.g. human or veterinary).

A handle 34 may extend from the main housing 12. The handle 34 may include a grip point for the hand and/or fingers of a user (e.g. recess or jug handle). The handle 34 may provide a natural holding or grasp point on the syringe pump 10 helping to direct users away from attempting to carry the syringe pump 10 by the drive head 38. The handle 34 may also act as a protection member as it may contact ground/objects first or in place of the drive head 38 in the event that the syringe pump 18 is dropped or otherwise inappropriately handled. Thus the handle 34 may assist in guarding against damage incurred by the drive head 38 or drive assembly 82 (see, e.g., FIGS. 38A-40, FIGS. 44A-49B, FIGS. 59A-E, FIGS. 60A-D, FIGS. 64A-C) during improper handling of the syringe pump 10. The handle 34 may also include a bearing surface 44 which interfaces with the drive head 38. The bearing surface 44 may guide and support the drive head 38 as the drive head 38 is displaced with respect to the main housing 12 (further described in relation to FIGS. 64A-C).

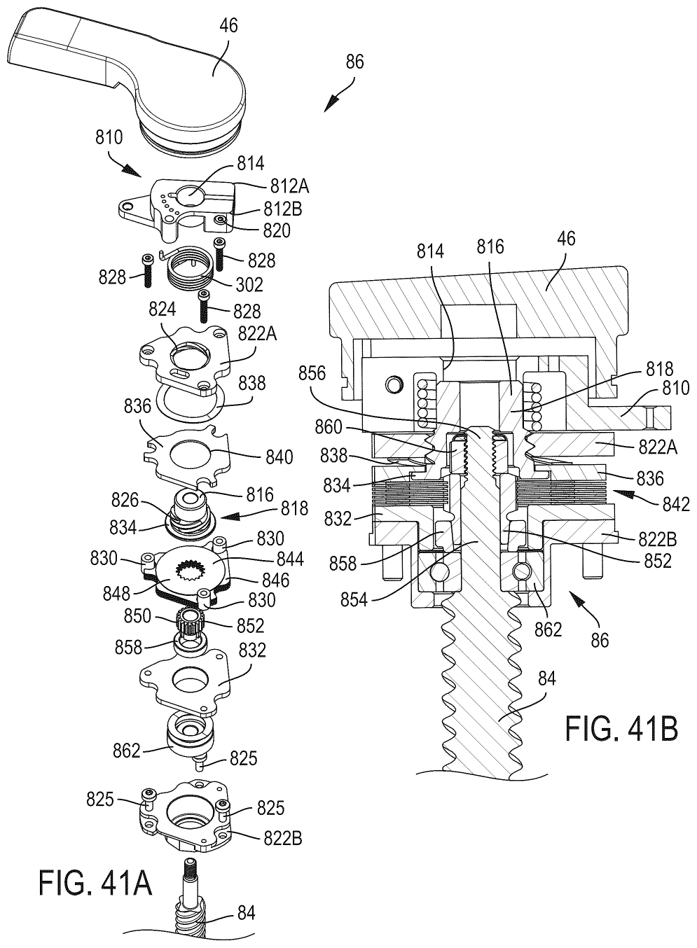

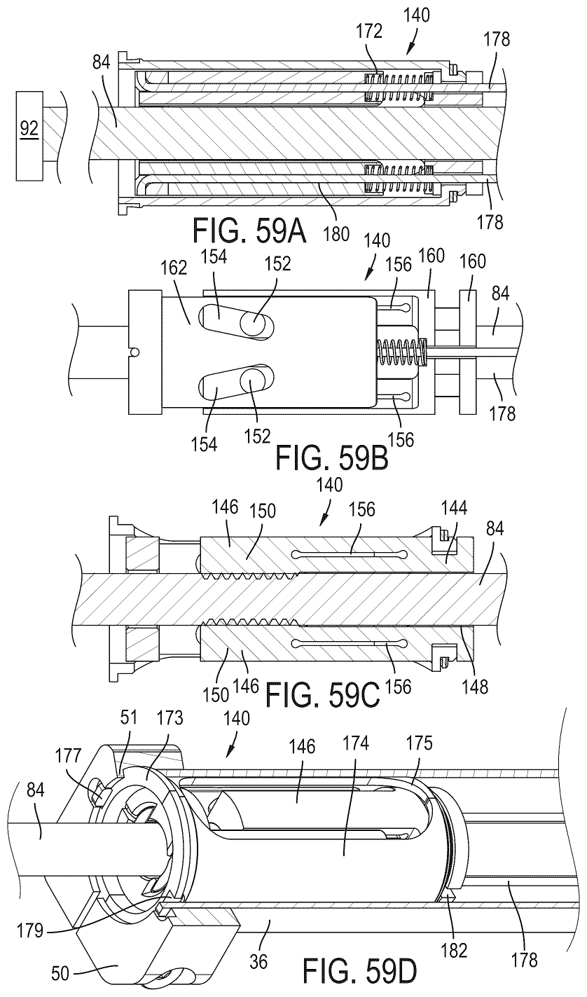

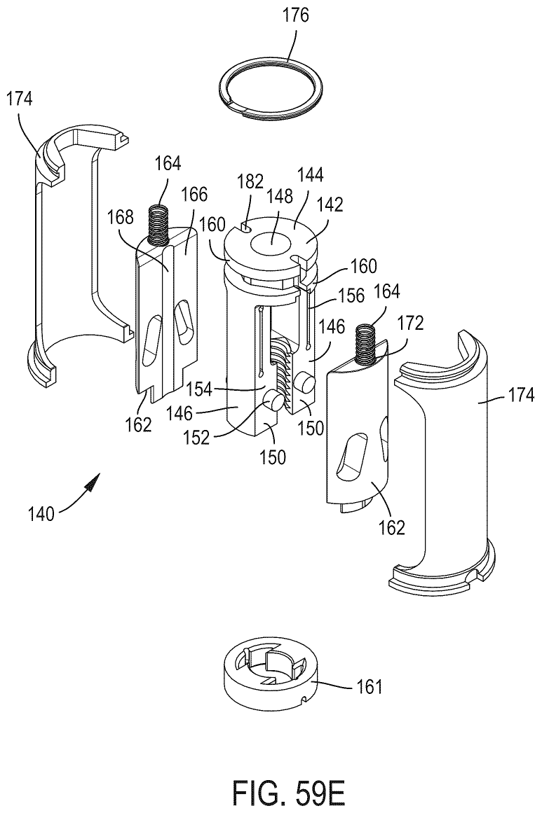

The syringe pump 10 may include an actuator 46 coupled to the drive assembly 82 (see, e.g., FIGS. 38A-40, FIGS. 44A-49B, FIGS. 59A-E, FIGS. 60A-D, FIGS. 64A-C). The actuator 46 may be operated to transition the drive assembly 82 to a released state in which the drive head 38 may be freely moved by a user. In the example shown, the actuator 46 is depicted as a lever body coupled to the drive head 38. The actuator 46 may be displaced (e.g. rotated) to disengage, for example, a clutch (e.g., any of those shown and described in relation to FIGS. 41A-43B, half-nut, collet nut (e.g., any of those shown and described in relation to FIGS. 59A-60D), etc. within the drive assembly 82.

Referring now to FIGS. 4-6B, various views of an example syringe retention assembly 19 are depicted. In various embodiments, syringe retention assemblies 19 may include a barrel grasper assembly 22 and optionally a barrel flange grasper assembly 30. In certain examples, the barrel flange grasper assembly 30 may be separate from the barrel grasper assembly 22 and, for example, be coupled to the main housing 12 of the syringe pump 10. Example barrel grasper assemblies 22 may include a set of barrel retainer bodies. At least a first and second barrel retainer body may be included. The barrel retainer bodies may be linked together by a linking assembly (e.g. set of gears and/or linkages) such that barrel retainer bodies displace in coordination with one another between a closed state and an open state. Displacement of one of the barrel retainer bodies may, via the linking assembly, engender displacement of at least one other barrel retainer body of the barrel grasper assembly 22. When the barrel retainer bodies are in the open state, the barrel 20 of syringe 18 may be placed between the barrel retainer bodies and the barrel retainer bodies may be displaced towards the closed state. As they barrel retainer bodies are displaced toward the closed state, the barrel contacting faces of the barrel retainer bodies may come into abutment with the exterior surface of the barrel 20 of the syringe 18. The barrel contacting faces may be shaped such that the syringe barrel 20 of the syringe 18 is aligned with a fiducial reference axis A1 is held in a retained state within the barrel grasper assembly 22 by the barrel retainer bodies.

As shown in FIGS. 4-6B, the exemplary syringe retention assembly 19 is depicted with a drive head tube exit panel 56 of the main housing 12 of the syringe pump 10. The syringe retention assembly 19 may include a backing panel 58. A barrel grasper assembly 22 of the syringe retention assembly 19 may include a set of jaws 60A, B which may act as the barrel retainer bodies described above. One of the jaws 60B, may include an extension portion 62 which may be shaped to facilitate grasping of the jaw 60B between, for example, a thumb and finger. The jaws 60A, B may be geared to one another such that movement of one jaw 60A, B engenders movement of the other jaw 60A, B. As one jaw 60A, B is displaced, the other of the jaws 60A, B may displace in an opposing manner. The jaws 60A, B may be coupled to one or more bias member (e.g. torsion spring) which may urge the jaws 60A, B toward one another and to a closed or syringe 18 retaining state. To install or remove a syringe 18, a user may grasp the extension portion 62 and displace the jaws 60A, B such that they spread apart to release the syringe 18. With the jaws 60A, B in the open state, a syringe 18 may be placed between the jaws 60A, B and the extension portion 62 may be released. The at least one bias member may drive the jaws 60A, B against the barrel 20 of the syringe 18 to ensure that the syringe 18 is firmly held in place within the jaws 60A, B.

As mentioned above, the barrel grasper assembly 22 may center the axis of the bore of the barrel 20 of the syringe 18 on a fiducial reference axis A1 (see, e.g., FIG. 1). As best shown in FIG. 6A-B, the jaws 60A, B of the barrel grasper assembly 22 may be shaped such that this centering occurs regardless of the syringe 18 size. One of the jaws 60A may be arranged to contact the syringe 18 at substantially a single point which may be located at a tip region 61 of the jaw 60A. The other of the jaws 60B, may be provided with two curved syringe contacting surfaces 63A, B. One may have a convex curvature while the other may have a concave curvature. As shown, a curved transition region may separate the two syringe contacting surfaces 63A, B. Each curved syringe contacting surface 63A, B of jaw 60B may contact a retained syringe 18 substantially at one point. The curves of the syringe contacting surfaces 63A, B may be derived such that the point along each syringe contacting surface 63A, B that contacts the syringe 18 changes depending on the syringe 18 while keeping the center axis of the bore in a constant location independent on the type of syringe 18 being retained. In various examples, the contact points between the jaws 60A, B and the syringe 18 may be approximately 120° apart from one another when a syringe 18 is retained. The profile of the curved syringe contacting surfaces 63A, B of jaw 60B may be dependent upon the range of syringe 18 diameters intended to be accepted by the barrel grasper assembly 22. The profile of the curved syringe contacting surfaces 63A, B may also be dependent upon the gearing ratio between the jaws 60A, B.

At least one of the jaws 60A, B may be constructed at least partially of a translucent or a transparent material. In the example, a section 64 of jaw 60B is formed of transparent material. This may facilitate viewing of any markings (e.g. graduations) printed on the exterior of the barrel 20 of the syringe 18. Other barrel retaining bodies described herein may include similarly transparent or translucent portions.

The position of at least one of the jaws 60A, B may be monitored by one or more retaining body position sensor 73. The sensor 73 may output a data signal which varies in relation to the position of the at least one of the jaws 60A, B. A controller 15 may analyze data from the sensor 73 to determine information related to the syringe 18. For example, the controller 15 may utilize information from the retaining body position sensor 73 to determine whether a syringe 18 is in place in the barrel grasper 22 assembly or properly retained by the barrel grasper assembly 22. The controller 15 may also utilize information from the sensor 73 to determine the size of the barrel 20 of the syringe 18. The type of syringe 18 installed on the syringe pump 10 may be determined by the controller 15 based at least in part on positional data for the jaws 60A, B received from the sensor 73. The sensor 73 may, for example, track the rotational displacement of one of the jaws 60A, B. The amount of rotational displacement or rotational position may be used to determine the type of syringe 18 being retained (e.g. by providing data related to the barrel 20 diameter). This may be used for a variety of purposes (further described elsewhere herein) such as facilitating pressure monitoring within the syringe 18 during infusion based on the internal diameter and piston area of the identified syringe 18.

A barrel flange grasper assembly 30 also may be included in an example syringe retention assembly 19 and may be coupled to the barrel grasper assembly 22. In the example shown, when a syringe 18 is installed, the barrel flange 32 of the syringe 18 may be disposed between an end of the barrel grasper assembly 22 and a clip 66 of the barrel flange grasper 30. The syringe retention assembly 19 may, in certain examples include a post 68 (e.g. on a jaw 60B) which extends into a notch in the clip 66. A pivot pin may be inserted through a portion of the clip 66 and extend through the post 68 such that the clip 66 may be pivotally displaced with respect to the barrel grasper assembly 22. The clip 66 may be biased by at least one bias member against the barrel grasper assembly 22 or towards a flange retaining position. For example, barrel flange grasper 30 may include a spring loaded rod 70. The spring loaded rod 70 may extend through an aperture in the clip 66 and include a head portion which is larger than the dimensions of the aperture. As the clip 66 is pivoted away from the barrel grasper assembly 22, the clip 66 may push against the head of the rod 70 and displace the rod 70. This may store a bias in the bias member associated with the rod 70. The bias member may ensure that the clip 66 is seated against the barrel flange 32 and held firmly in place once the syringe 18 is installed. The clip 66 may include a chamfered or rounded edge 69 (best shown in FIG. 4) to facilitate pivotal displacement of the clip 66 as the barrel flange 32 is pressed into position. Thus, the clip 66 may not need to be manually held open to position the barrel flange 32.

In certain examples, a sensor 71 may monitor the position of the rod 70. A controller 15 may analyze data from the sensor 71 to determine information related to the syringe 18. For example, the controller 15 may utilize information from the rod position sensor 71 to determine whether a barrel flange 32 is in place in the barrel flange grasper assembly 30 or properly retained by the barrel flange grasper assembly 30. In such examples, a sensor 71 which outputs a binary type reading (e.g. microswitch) may be used. The sensor 71 may output a first signal when the rod 70 is in a resting position and a second signal when the rod 70 has been disturbed from the resting position (e.g. due to displacement of the clip 66). Receipt of the second signal by the controller 15 may indicate that the clip 66 is raised and pressing against a barrel flange 32. In some embodiments, when the retaining body position sensor 73 of the barrel grasping assembly 22 indicates a syringe 18 is retained, the clip 66 may be interpreted by the controller 15 to be in proper retaining position against a barrel flange 32 when the sensor 71 indicates the clip 66 is raised. In alternative embodiments, the controller 15 may also utilize information from the sensor 71 to determine the size of the barrel 20 of the syringe 18 (e.g. inferred by collecting data related to the thickness of the barrel flange 32). In such embodiments, any suitable sensor 71 for tracking axial displacement of the rod 70 may be used. The type of syringe 18 installed on the syringe pump 10 may be determined based at least in part on positional data for the rod 70. As mentioned above, this may be utilized for a variety of purposes as described elsewhere herein.

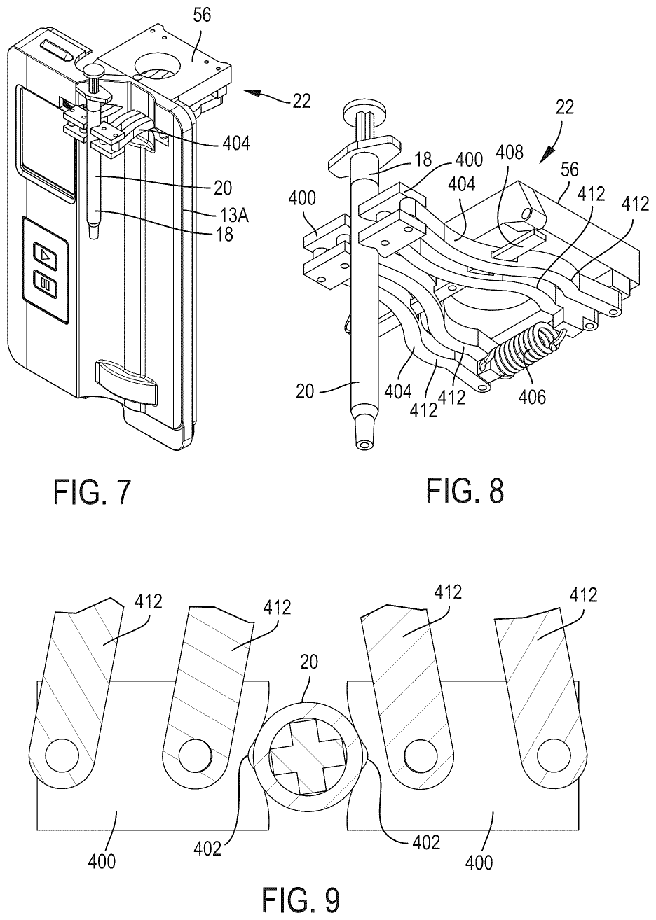

Another example barrel grasper assembly 22 is depicted in FIGS. 7-9. The retaining bodies for the example barrel grasper assembly 22 may include a set of opposing retainers 400. Each retainer 400 may include a trough 402 which may be defined as a V shaped recess. The retainers 400 may each be attached to a terminal end of respective arm assemblies 404. Each arm assembly 404 may include a set of elongate bodies 412. The elongate bodies 412 may extend along a curved or redirecting path so as to surround but not contact a drive head tube 36 of the syringe pump 10 extending through the drive head tube exit plate 56. Ends of the elongate bodies 412 opposite the retainers 400 may be pivotally coupled to the drive head tube exit plate 56 (or some other stationary part of the syringe pump 10 such as the housing 12).

The arm assemblies 404 may be displaceable toward and away from one another to transition the barrel grasper assembly 22 between and open and a closed state. A sealing member (e.g. accordion seal) between arm assemblies 404 and the housing 12 may be included and may ensure the housing 12 remains sealed as the arm assemblies 404 displace. In the example embodiment, the arm assemblies 404 may be biased toward the closed state by one or more bias member 406 which may, for example, be an extension spring as shown. The arm assemblies 404 may be coupled to one another via linking assembly including one or more linkage and/or gearing arrangement such that motion of one arm assembly 404 in a first direction may engender motion of the opposing arm assembly 404 in a second and opposite direction.

Each arm assembly 404 may include a passage 410 extending therethrough. A guide 408 may be included in the barrel grasper assembly 22 and in the example embodiment. As shown, the guide 408 is depicted as a bar having portions which extend through the passages 410 in each arm assembly 404. The guide 408 is shown coupled to the drive head tube exit panel 56. As the arm assemblies 404 are displaced, the guide 408 may assist in constraining the displacement path of the arm assemblies 404 such that the retainers 400 displace substantially along mirrored displacement paths. The displacement path of the retainers 400 may be substantially lateral with respect to any syringe 18 being introduced into the barrel grasper assembly 22. As the arm assemblies 404 are displaced toward the closed position, the retainers 400 may contact an exterior the barrel 20 of a syringe 18 to be retained. As the arms assemblies 404 displace in tandem with one another, the syringe 18 may be contacted in a central region between the retainers 400 of the barrel grasper assembly 22. As best shown in FIG. 9, the shape of the trough 402 of each retainer 400 may adjust the position of the barrel 20 of the syringe 18 such that the bore of the barrel 20 is centered between the retainers 400. Since the movement of the retainers 400 is substantially lateral to the syringe 18, the retainers 400 may also adjust the axis of the barrel 20 of the syringe 18 to align closely with a fiducial reference axis A1 (see, e.g., FIG. 1) due to the bias exerted by the bias member 406. The bias member 406 may also exert a force against the arm assemblies 404 which keep the retainers 400 firmly pressing against the barrel 20 holding the syringe 18 in a retained position within the barrel grasper assembly 22. One or more sensor may be included to determine the size of the syringe 18 barrel 20 based on the position of the arm assemblies 404. For example, a rotational sensor (e.g. potentiometer) may monitor the rotational position of one of an arm assembly 404. When a syringe 18 is installed, the position of the arm assembly 404 may differ depending on the size of the syringe 18 and thus the output signal of the sensor may be used be a controller 15 of the syringe pump 10 to determine the type of syringe 18 being retained. Gearing and/or linkage(s) may be included to tie motion of the arm assemblies 404 together such that they move in a defined relationship relative to one another (e.g. such that each arm assembly 404 displaces an equal amount in opposing directions).

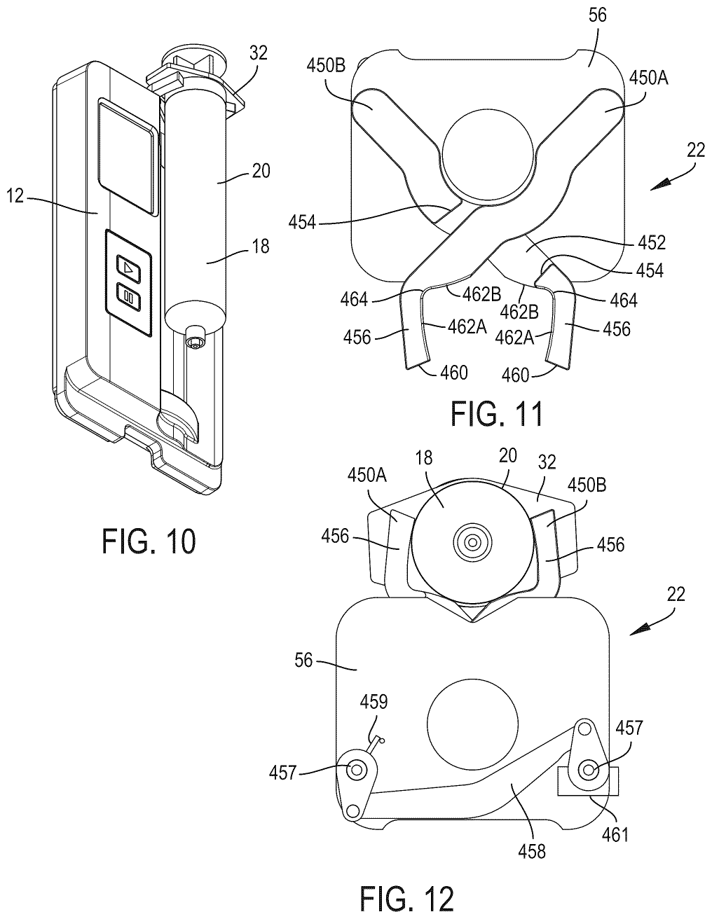

Referring now to FIGS. 10-12, another exemplary barrel grasper assembly 22 is depicted. As shown, the barrel retaining bodies of the example barrel grasper assembly 22 include a set of jaws 450A, B which are pivotally coupled to the drive head tube exit panel 56 at one end. Each jaw 450A, B may include an opposing end region 456 configured to contact the barrel 20 of a syringe 18. The jaws 450A, B may be arranged so as to cross one another. In the example embodiment, each jaw 450A, B includes a notch 452 which accepts a crossing portion of the opposing jaw 450A, B. The jaws 450A, B may be displaced with respect to one another in a scissoring motion such that the gap between the end regions 456 of each jaw 450A, B may be adjusted. The jaws 450A, B may be displaceable between an open or fully spread apart state and a closed state or state in which the end regions 456 of the jaws 450A, B are most proximal to one another. The jaws 450A, B may be biased by one or more bias member toward the closed state. For example, a torsion spring 459 may be associated with a pivot pin 457 for at least one of the jaws 450A, B. The sidewalls 454 of the notches 452 may form stops which limit the range of motion of the jaws 450A, B. Additionally, as best shown in FIG. 12, a linking assembly may be included. As shown, each jaw 450A, B may be coupled to the opposing jaw 450A, B via a linkage arrangement 458. Thus, motion of the jaws 450A, B may be tied together and coordinated. Displacement of one of the jaws 450A, B may result in motion of the other jaw 450A, B in an opposing direction. Rotation of at least one of the jaws 450A, B about its pivot axis may be monitored by a sensor 461 (see also, e.g., retaining body position sensor 73 of FIG. 4) which may output a signal which varies in relation to the rotational position of the jaw 450A, B. This output may be used to determine the type of syringe 18 installed in the barrel grasper assembly 22 based on the diameter of the syringe 18.

Each end region 456 may include a guide face 460 at its terminal end. As a syringe 18 is pressed against the guide faces 460 when the jaws 450A, B are in the closed state, the angle on the guide faces 460 may direct the barrel 20 of the syringe 18 into a region between the end regions 456 of the jaws 450A, B. Additionally, as the syringe 18 is advanced against the jaws 450A, B, the angle on the guide faces 460 may facilitate spreading of the jaws 450A, B as the syringe 18 is advanced against the guide faces 460. When the barrel 20 of the syringe 18 is in place within the space between the end regions 456, the at least one bias member of the barrel grasper assembly 22 may firmly hold the end regions 456 in contact with the barrel 20 to retain the syringe 18.

The end regions 456 of the jaws 450A, B may be shaped such that the bore of the barrel 20 of any accepted syringe 18 size is centered on an axis (e.g. fiducial reference axis A1 of FIG. 1) as the end regions 456 of the jaws 450A, B press against the barrel 20 of the syringe 18. Each end region 456 may contact the barrel 20 of an installed syringe 18 substantially at two points and may include two barrel contacting faces 462A, B which are separated by a curved transition region 464. The barrel contacting faces 462A at the tips of the end regions 456 may arc in a generally concave manner. The barrel contacting faces 462B inward from the tips may arc in a generally convex manner. The profiles of the barrel contacting faces 462A, B may be dictated by the range of syringe 18 barrel 20 diameters intended to be accepted by the barrel grasper assembly 22.

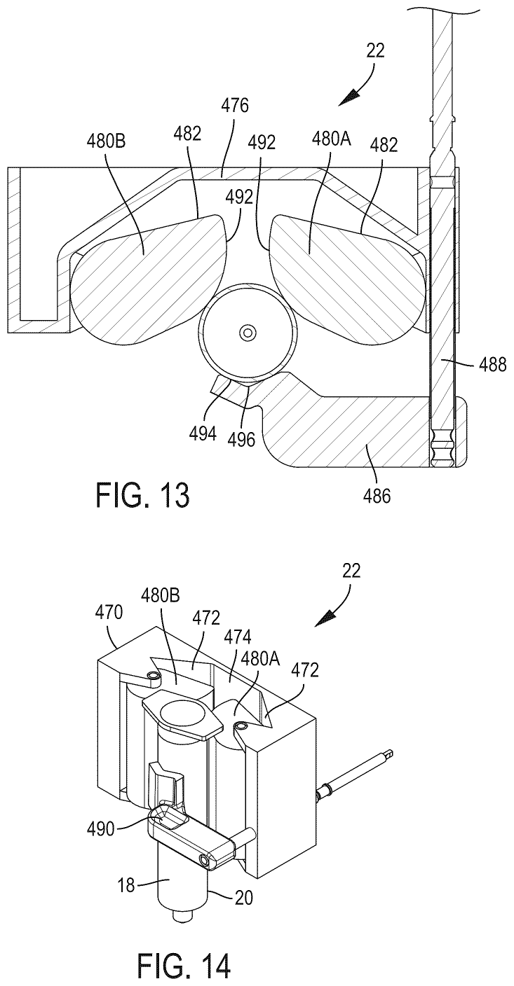

Referring now to FIGS. 13-14 another exemplary embodiment of a barrel grasper assembly 22 is depicted. As shown, the barrel grasper assembly 22 may include a mounting body 470. The housing 12 for a syringe pump 10 may include a receptacle to which the mounting body 470 may couple when the syringe pump 10 is fully assembled or the mounting body 470 may be formed as part of the housing 12 in certain examples. The barrel retaining bodies in the example embodiment are shown as a set of cam jaws 480A, B and a retainer 486 of a barrel clamp assembly 484. The cam jaws 480A, B may be coupled to the mounting body 470 at opposing sides of a central recess 476 in the mounting body 470. The cam jaws 480A, B may each be pivotally coupled to the mounting body 470 so as to be rotatable toward and away from the surface of the recess 476. Each cam body 480A, B may include a flat face 482 most proximal the surface of the recess 476. The recess 476 may be defined by two angled surfaces 472 which flank a central, substantially planar region 474 of the recess 476. The flat faces 482 may abut the angled surfaces 472 of the mounting body 470 at an end of the displacement range of each cam body 480A, B such that the angled surfaces 472 may provide stops for the cam jaws 480A, B. A bias member (e.g. one or more torsion spring) may be associated with each of the cam jaws 480A, B and may urge the cam jaws 480A, B to the opposite end of their displacement range. Each cam jaw 480A, B may also include a cam face 492.

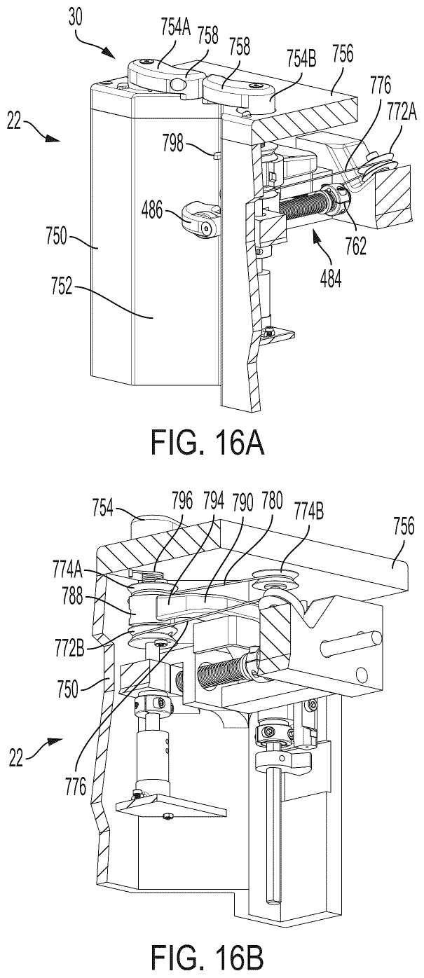

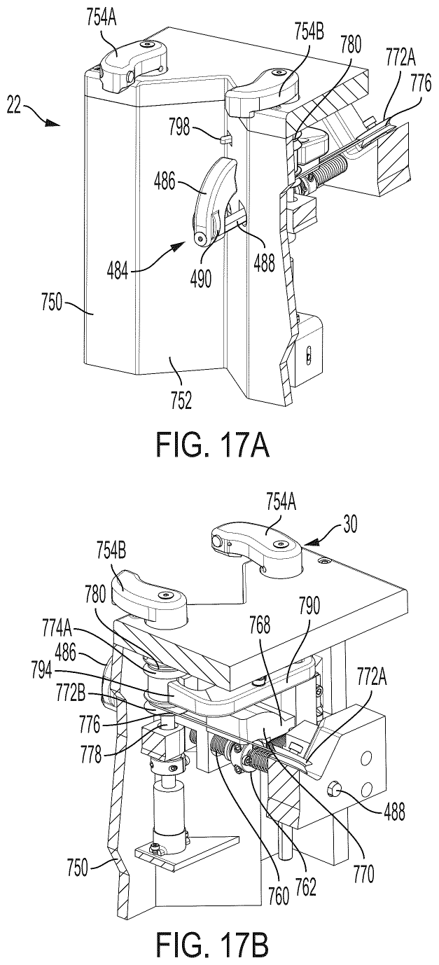

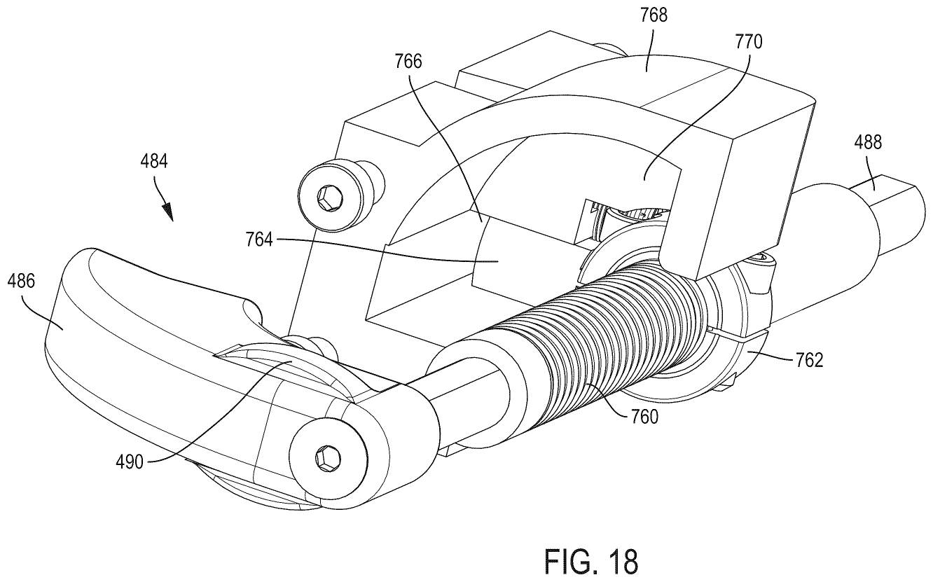

The barrel grasper assembly 22 may also include a barrel clamp assembly 484. Only a portion of a barrel clamp assembly 484 is depicted in FIGS. 13-14. An example barrel clamp assembly 484 is further described in relation to FIGS. 16A-21B. A barrel clamp assembly 484 may include a retainer 486 which may be attached to the terminal end of a rod 488. The retainer 486 may include a grip region 490 which may be grasped by a user to displace the retainer 486 with respect to the mounting body 470. In the example, the retainer 486 may be translationally displaced parallel to the axis of the rod 488 and may be rotationally displaced about the axis of the rod 488. The retainer 486 may include a barrel contacting face 494. The barrel contacting face 494 may include a recess 496 which may be V-shaped. The barrel clamp assembly 484 may include a bias member (see, e.g., bias member 760 of FIG. 17B) which may urge the retainer 486 in the direction of the main body 470. The bias member may help ensure that the retainer 486 firmly holds the syringe 18 in place against the cam jaws 480A, B.

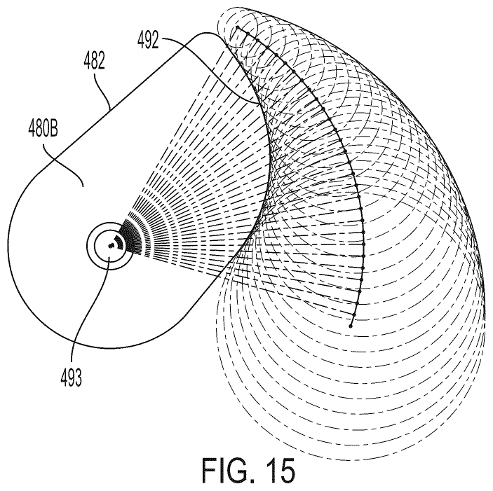

Referring now also to FIG. 15, the cam faces 492 may be shaped such that each cam jaw 480A, B may accept any of a variety of syringe 18 barrel 20 diameter sizes while still keeping the center of the bore of the barrel 20 aligned with an axis (e.g. the fiducial reference axis A1 described in relation to FIG. 1). As shown, to determine the profile of the cam face 492, an arc may be created at a constant radius from a pivot point 493 of a cam body 480A, B. Circles having evenly incremented increasing diameters within the range of diameters allotted for use with the barrel grasper assembly 22 may be placed at regular increments along the arc. The profile of the cam face 492 may be formed by a curved line running tangent to the edge of each of the circles.

When installed, the retainer 486 may hold the barrel 20 of the syringe 18 in contact with each of the cam jaws 480A, B. The shape of the recess 496 may locate the syringe 18 in a central location between the cam jaws 480A, B. Each cam jaw 480A, B may contact the barrel 20 of the syringe 18 at substantially a single point. The rotational position of the cam jaws 480A, B may differ depending on the diameter of the syringe 18 barrel 20. The cam jaws 480A, B may be coupled to one another via a linking assembly which ensures that the cam jaws 480A, B rotate in a defined relation with respect to one another (e.g. rotate in equal amounts in opposing directions). Gearing, pulleys (see, e.g., the arrangement linking the set of arms 754A, B in FIGS. 16A-21B), or some combination thereof may be used in various examples. The rotational position of the cam jaws 480A, B may be monitored by at least one sensor. The output of the sensor may be used by a controller 15 of the syringe pump 10 to determine the type of syringe 18 installed in the barrel grasper assembly 22. Each of the cam jaws 480A, B may be associated with a bias member (e.g. torsion spring) which may bias the cam jaws 480A, B toward an end of their displacement range opposite the angled surfaces 472.

Referring now to FIGS. 16A-21B another example embodiment of a syringe retention assembly 19 is depicted. The example syringe retention assembly 19 includes a barrel grasper assembly 22. Referring primarily to FIGS. 16A-B, the barrel grasper assembly 22 may include a panel 750 with a trough 752. The trough 752 may be shaped substantially in the form of the Latin character “V”. Though the syringe retention assembly 19 is depicted in isolation, the panel 750 may be included as a portion of the housing 12 of an example syringe pump 10. When a syringe 18 is retained by the barrel grasper assembly 22, the syringe 18 may be placed in the trough 752. The center axis of the syringe 18 may vary depending on the syringe installed 18 with smaller syringes 18 sitting deeper in the trough 752 than larger syringes 18. The barrel grasper assembly 22 may include a barrel clamp assembly 484 which may hold the syringe 18 in place when a syringe 18 is retained by the barrel grasper assembly 22.

The example syringe retention assembly 19 also includes a barrel flange grasper assembly 30. The barrel flange grasper assembly 30 may retain and locate a barrel flange 32 of an installed syringe 18 in place against the barrel grasper assembly 22. The barrel flange grasper assembly 30 may include a set of arms 754A, B which may be pivotally coupled with respect to a second panel 756 of the barrel grasper assembly 22. When a syringe 18 is installed the arms 754A, B may be closed over the barrel flange 32 to hold the barrel flange 32 in place against the panel 756. Though shown isolated from a syringe pump 10 in FIGS. 16A-21B, the panel 756 may also form or be incorporated into the housing 12 of a syringe pump 10. The set of arms 754A, B may be coupled to the barrel clamp assembly 484 such that displacement of the retainer 486 of the barrel clamp assembly 484 may pivotally displace the arms 754A, B. Other syringe retention assemblies 19 described herein may have a barrel clamp assembly 484 linked to rotatable components of the respective syringe retention assemblies 19 similarly to as described in relation to FIGS. 16A-21B. Thus, for example, cam jaws 480A, B of FIGS. 13-15 may be driven by displacement of a barrel clamp assembly 484.

As shown in FIGS. 16A-B, the barrel clamp assembly 484 may be biased to a closed state. When no syringe 18 is present, a retainer 486 of the barrel clamp assembly 484 may be against the trough 752 of panel 750. The arms 754A, B of the barrel flange grasper assembly 30 may also be biased (by bias members 796 such as torsion springs) towards a closed state in which the grasping ends 758 of the arms 754A, B are swung towards the deepest part of the trough 752.

Referring now to FIGS. 17A-B, the barrel clamp assembly 484 may include a rod 488 which extends through the panel 750. The exterior end of the rod 488 may include a retainer 486 with a grip region 490. A user may grasp the grip region 490 and pull the rod 488 such that it is advanced out of the panel 750. A bias member 760 may be disposed around the rod 488 between the rear face of the panel 750 and a mounting body 762 attached to the rod 488. The bias member 760 may become compressed as the mounting body 762 is displaced toward the panel 750 while a user pulls on the retainer 486.

Referring now also to FIG. 18, a perspective view of an example barrel clamp assembly 484 is depicted. The mounting body 762 may include a projection 764 attached thereto which may displace along a guide 766. The interaction of the guide 766 and the projection 764 may prevent rotation of the rod 488 about the axis of the rod 488 while the projection 764 is disposed within the guide 766. As the retainer 486 is displaced toward an end of its displacement range, movement of the rod 488 and mounting body 762 may advance the projection 764 beyond the end of the guide 766. As shown in FIGS. 17A-B, the rod 488 and attached components may then be rotated about the axis of the rod 488. A latch 768 may be included in the barrel clamp assembly 484 and may inhibit rotation in certain directions or limit the range of available displacement. In the example embodiment, the latch 768 allows the projection 764 to swing in an arc which provides for the retainer 486 to rotate substantially 90°. Thus, the retainer 486 may be displaced clear of the trough 752 to facilitate installation of a syringe 18 in the barrel grasper assembly 22. The latch 768 may also include a stop surface 770. When the rod 488 is rotated, the projection 764 coupled to the mounting body 762 may be swung in an arc. When released, the bias member 760 may urge the rod 488 to retract as it presses against the mounting body 762. The projection 764 may catch on the stop surface 770 to hold the barrel clamp assembly 484 in an open state freeing both of a user's hands to manipulate a syringe 18 for installation.

As mentioned above, motion of the arms 754A, B of the barrel flange grasper assembly 30 may be tied to displacement of the barrel clamp assembly 484. Example syringe retention assemblies 19 may include a first set of pulleys 772A, B and a second set of pulleys 774A-C. The first set of pulleys 772A, B may be associated with a line 776. The line 776 may be any suitable line 776 such as a flexible cord, cable, wire, belt, etc. The line 776 may be anchored to the mounting body 762 on one end. The line 776 may extend from the mounting body 762 and wrap around pulley 772A of the first set of pulleys 772A, B. From pulley 772A, the line 776 may extend around pulley 772B. The end of the line 776 opposite that anchored to the mounting body 762 may be anchored to pulley 772B. Pulley 772B may be coupled to a pivot pin 778 to which arm 754B is coupled and about which arm 754B may pivot.

As a user advances the rod 488 out of the panel 750 by pulling on the retainer 486, the end of the line 776 anchored to the mounting body 762 may be displaced in tandem with the rod 488. Pulley 772B and the attached pivot pin 778 may rotate to spool out line 776 to facilitate displacement of end of the line 776 anchored to the mounting body 762. This, in turn may drive rotation of the arm 754B away from the trough 752 to an open state.

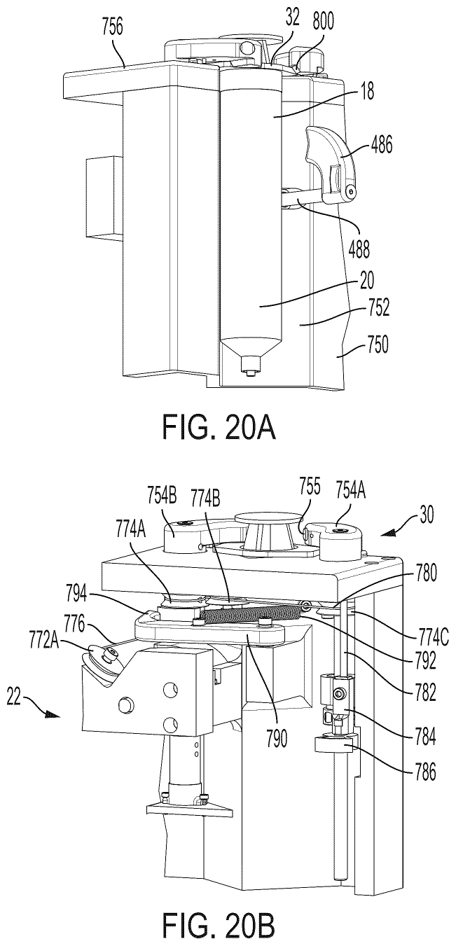

The second set of pulleys 774A-C may ensure that both arms 754A, B rotate to an open state in tandem with one another. Pulley 774A may be coupled to the pivot pin 778 so as to rotate together with the pivot pin 778. As shown, a second line 780 is included and may have an end anchored to pulley 774A. The second line 780 may extend from pulley 774A around pulley 774B (see, e.g., FIG. 20B) and to pulley 774C. An end of the second line 780 may also be anchored to pulley 774C (see, e.g., FIG. 20B). Pulley 774C may be coupled to and rotate with a second pivot pin 782 which forms the rotation axis for arm 754A. As the first pivot pin 778 is driven via the first set of pulleys 772A, B in response to the barrel clamp assembly 484 being actuated, pulley 774A may rotate and take up some of line 780. In order to accommodate this, pulley 774C may rotate to spool out some of line 780 wrapped around that pulley 774C. As pulley 774C rotates, the second pivot pin 782 and arm 754A will rotate. Thus both arms 754A, B will open in unison as the barrel clamp assembly 484 is actuated to an open state.

As best shown in FIG. 20B, a sensor 784 may be included to monitor the rotational orientation of at least one of the pivot pins 778, 782. For example, a sensor 784 may monitor the position of a flag 786 that rotates with the second pivot pin 782. A signal from the sensor 784 may be analyzed by a controller 15 to track the position of the arms 754A, B. Such a sensor arrangement may be adapted to sense rotation of any rotationally displaceable components described in relation to other syringe retention assemblies 19 described herein.

Referring primarily to FIGS. 16A-17B, a ratchet body 788 may be included on one of the pivot pins 778, 782. The ratchet body 788 may rotate with the associated pivot pin 778. A pawl 790 may also be included. The pawl 790 may be biased against the ratchet body 788 by a bias member 792 (see, e.g., FIG. 20B). In the example shown, the ratchet body 788 is coupled to pivot pin 778 and rotates therewith. As the ratchet body 788 is rotated, a ledge on the ratchet body 788 may be displaced beyond a tooth portion 794 of the pawl 790. The bias member 792 may urge the tooth portion 794 to displace into engagement with the ledge of the ratchet body 788 inhibiting rotation of the pivot pin 778. This may hold the arms 754A, B in an open state against any bias presented by bias members 796 (e.g. torsion springs) associated with each of the pivot pins 778, 782 that would otherwise compel the arms 754A, B to a closed state.

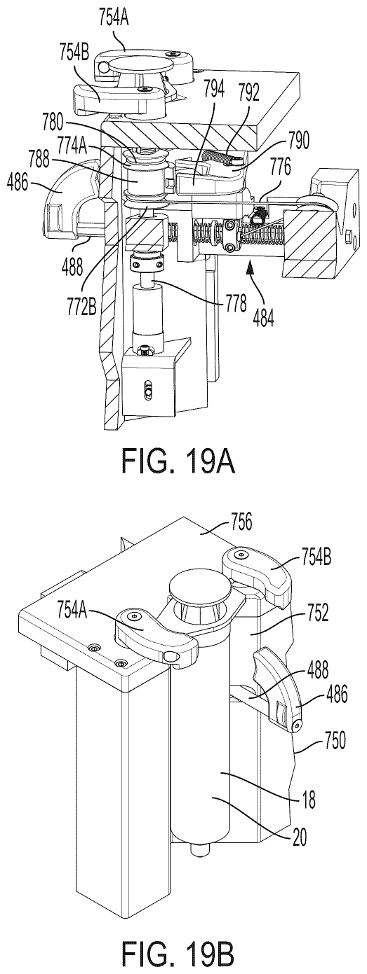

Referring now primarily to FIG. 17A and FIGS. 19A-B, the pawl 790 may also include a finger 798 which may extend through an aperture in the panel 750 and protrude proud of the surface of the trough 752. A seal (e.g. bellows type seal) may be provided around the finger 798. When syringe 18 installed, advancement of the syringe 18 into the trough 752 may displace the finger 798 into the panel 750. This may cause the pawl 790 to rotate such that the tooth portion 794 of the pawl 790 is driven out of engagement with the ratchet body 788 as shown best in FIG. 19A.

Referring now to FIG. 20A-B, with the pawl 790 disengaged from the ratchet body 788, bias members 796 (see, e.g., FIG. 16B) associated with each of the pivot pins 778, 782 may urge the pivot pins 778, 782 to rotate such that the arms 754A, B rotate closed against the barrel flange 32 of the installed syringe 18. Rotational dampers may be coupled to each pivot pin 778, 782 to encourage this motion to occur gently. Each of the arms 754A, B may include a flange contact face 800 which may include a bevel. As the arms 754A, B displace into contact with the barrel flange 32, the bevel of the flange contact faces 800 may press the barrel flange 32 firmly in place against panel 756. The position of the arms 754A, B may be sensed by a sensor 784 and data from the sensor 784 may be used by a controller 15 to infer the type of syringe 18 installed. This data may also be used by a controller 15 to indicate whether a flange 32 is being properly held by the barrel flange grasper assembly 30. If, for example, readings indicate the arms 754A, B are in a position outside of an expected range, the controller 15 may determine the syringe 18 has been misloaded and generate a notification (e.g. for display on a user interface 13B). At least one of the arms 754A, B may include a barrel detector 755. The barrel detector 755 may verify that the arms 754A, B have retained the barrel flange 32 instead of swung into the barrel 20 of a misloaded syringe 18. Any suitable sensor may be used such as a microswitch or button type switch which would be depressed as the arm 754A, B rotates into a barrel 20 of a syringe 18. The controller 15 may determine a misload has occurred in the event that such a switch is in a depressed state. Displacement of the retainer 486 may also be tracked with a linear position sensor (e.g. potentiometer or any of those described herein) that monitors the rod 488 of the barrel clamp assembly 484. This information may be utilized by the controller 15 to further assist in determining the type of syringe 18 installed. Similarly, the output signal of the linear position sensor tracking the retainer 486 position may be used to determine a misloading scenario has occurred (e.g. output signal indicates the retainer 486 is not in an appropriate position).

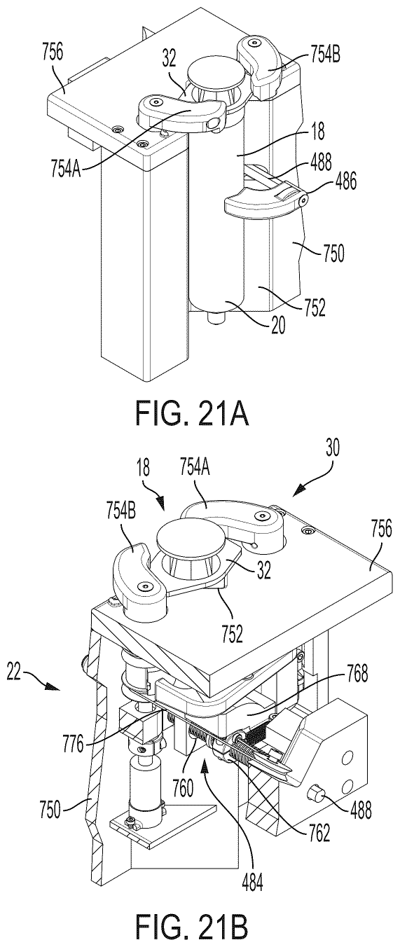

Referring now to FIGS. 21A-B, with the barrel flange 32 of the syringe 18 retained, the barrel clamp assembly 484 may be brought to a retaining state against the barrel 20 of the syringe 18. The retainer 486 may be grasped by a user and rotated such that a barrel contacting face 494 of the retainer 486 is positioned in alignment with the barrel 20. As the retainer 486 is rotated, the projection 764 may be rotated into alignment with the guide 766 and off of the stop surface 770 of the latch 768. The bias member 760 may press against the mounting body 762 and drive the rod 488 in a retraction direction as the bias member 760 restores to a less stressed state. This may urge the barrel contacting face 494 of the retainer 486 against the barrel 20 of the syringe 18 firmly holding the syringe 18 within the trough 752 of the panel 750.

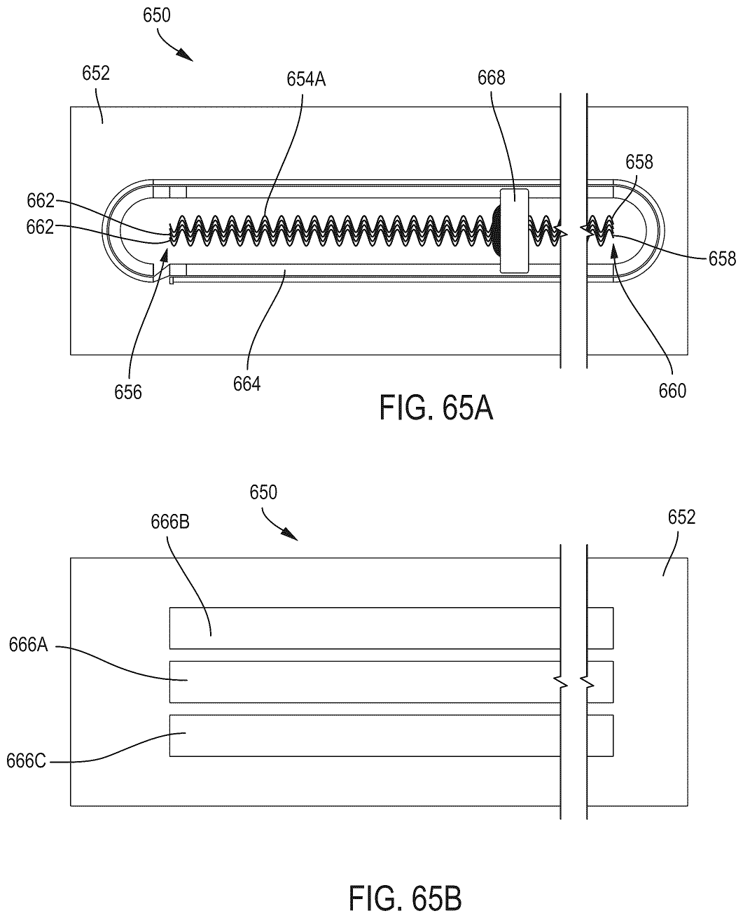

Still referring to FIGS. 21A-B, a linear position sensor 650 such as (though not limited to) one of the embodiments depicted in FIGS. 65A-73H may be used to monitor the position of a portion of the barrel clamp assembly 484. The rod 488 may retract a different amount under the restoring force of the bias member 760 depending on the diameter of the syringe 18 being retained. Data from the linear position sensor 650 may thus be used to determine the type of syringe 18 installed in the barrel grasper assembly 22.

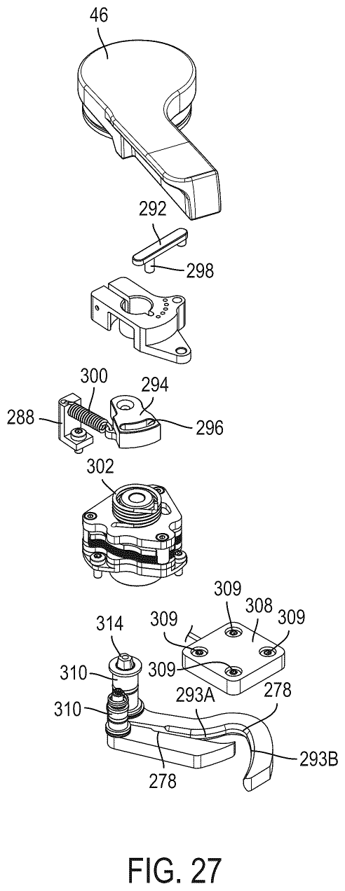

Referring now to FIGS. 22A-24, various views of an exemplary embodiment of a drive head 38 are depicted. As shown, example drive heads 38 may include a housing 250. Example drive heads 38 may also include a plurality of plunger flange grasper bodies 256 which may be pivotally displaceable about respective pivot axes. In certain examples, each plunger flange grasper body 256 may be coupled to a planetary gear 262 which may be engaged with a pivot body. In the example shown, each planetary gear 262 interdigitates with a pivot body in the form of a sun gear 264. The sun gear 264 is rotationally displaceable about an axis centrally disposed with respect to the pivot axes of the plunger flange grasper bodies 256. The sun gear 264 may include a slot 280. Exemplary drive heads 38 may also include an actuator 46 which is displaceable from a home position to an actuated position. The actuator 46 may be coupled to a linkage 272 including a projection (see, e.g., item 298 of FIG. 27) which extends into the slot 280. Displacement of the actuator 46 from the home position to the actuated position may be transmitted through the gearing arrangement to the plunger flange grasper bodies 256 to drive them from a closed position to an open position. The plunger flange grasper bodies 256 may be biased toward the closed position by at least one bias member 282. The actuator 46 may be biased to the home position by at least one additional bias member 284. When the actuator 46 is released by a user, it may, under power of the additional bias member(s) 284 be driven back to the home position. In the event that a plunger flange 28 is present between the plunger flange grasper bodies 256, the plunger flange grasper bodies 256 may be driven into the plunger flange 28 retaining it in place. This may inhibit the plunger flange grasper bodies 256 from fully returning to the closed position. The slot 280 in the sun gear 264 may allow for travel of the actuator 46 and attached linkage independent of displacement of sun gear 264 when the plunger flange grasper bodies 256 are blocked from fully reaching the closed position. That is, the slot 280 may allow clearance for the projection on the linkage 272 to displace absent corresponding displacement of the sun gear 264 as the actuator 46 is urged toward the home position by the bias member 284.

Still referring to FIGS. 22A-24, in some embodiments, the housing 250 may include a first housing section 252A and a second housing section 252B. The first housing section 252A, may include an aperture 254 through which the drive head tube 36 is coupled to the drive head 38. The drive head 38 may include an actuator 46 (shown as a lever in the example embodiment). The second housing portion 252B may include a recess providing clearance for the actuator 46 to be displaced. The actuator 46 may be actuated to allow a user to freely displace the drive head 38. As further described elsewhere herein, the actuator 46 may, for example, disengage a clutch (see, e.g., FIGS. 41A-4B or alternatively disengage a nut assembly 140 (see, e.g., FIGS. 59A-60D) from a leadscrew 84.

In the exemplary embodiment, the drive head 38 includes a set of three plunger flange grasper bodies 256. In other embodiments, a greater number of plunger flange grasper bodies 256 may be used. Each plunger flange grasper body 256 may be coupled to a pivot pin 258 which may extend through the first housing portion 252A. The pivot pin 258 may be coupled to an end region of the respective plunger flange grasper body 256. Each plunger flange grasper body 256 may be pivotally displaceable about the axis of its respective pivot pin 258. The axes of the pivot pins 256 may be disposed at regular angle intervals (e.g. 120° where three plunger flange grasper bodies 256 are included) and may surround a plunger flange receiving region 276 defined on the housing 250. A contact 260 (pin, plate, projection, button, or the like) which extends through the housing 250 may be disposed equidistant to each of the pivot axes, centered between the plunger flange grasper bodies 256. The contact 260 may be part of a sensing assembly 308 (see, e.g., FIG. 26B) and may be positioned to transmit force applied to the contact 260 to a load cell 330 (see, e.g., FIG. 32). In operation, the contact 260 may transfer force from an installed syringe 18 to the load cell 330 such that this force may be monitored. The plunger flange grasper bodies 256 may be displaced through a displacement range from a fully closed position to a fully open position. When the actuator 46 is actuated such that the drive head 38 may be manually displaced by a user (e.g. to remove a depleted syringe 18 and/or install a new, filled syringe 18), the plunger flange grasper bodies 256 may be displaced to the fully open position (see, e.g., FIG. 22B). This may help facilitate installation or removal of a syringe 18.

In the example, a grasper gear 262 is coupled to each of the pivot pins 258 at a position internal to the housing 250 (e.g. opposite the plunger flange grasper bodies 256). In the example embodiment, each of the grasper gears 262 is a planetary gear which may interdigitate with the sun gear 264 of the gearing arrangement. The sun gear 264 may rotate about an axis which extends through the contact 260 and is centrally disposed with respect to the pivot axes of the pivot pins 258. Each grasper gear 262 may be the same. Thus, for a given amount of rotation in the sun gear 264, each grasper gear 262 and plunger flange grasper body 256 may rotate the same amount. With the plunger flange grasper bodies 256 in the fully open state, a plunger flange 28 of any accepted syringe 18 may be placed within a plunger flange receiving area 276. Due to the planetary gearing arrangement, as the plunger flange grasper bodies 256 are displaced toward their closed positions, they may contact the plunger flange 28 and center the plunger flange 28 within the plunger flange receiving area 276. This may occur independent of the size of the plunger flange 28.

As mentioned above, when a plunger flange 28 is captured, the plunger flange grasper bodies 256 may be blocked from returning to a fully closed position. The sun gear 264 may include a notch 286 to provide clearance for a bias member 282 which may exert a force urging the plunger flange grasper bodies 256 to the closed position. When the plunger flange grasper bodies 256 are blocked from returning to the closed position, the bias member 282 may exert a force through the gearing arrangement which robustly holds the plunger flange grasper bodies 256 in place against the plunger flange 28. Additionally, the slot 280 may provide a clearance path which allows the actuator 46 and linkage 272 to return fully to the home position as the additional bias member 284 restores. Thus, any clutch (see, e.g., FIGS. 41A-43B) or nut assembly 140 (see, e.g., FIGS. 59A-60D) actuated by the actuator 46 may be re-engaged with a leadscrew 84 of the drive arrangement 82. As shown, the bias members 282, 284 are extension springs which are coupled to anchor posts 288 formed in or coupled to the housing 250.

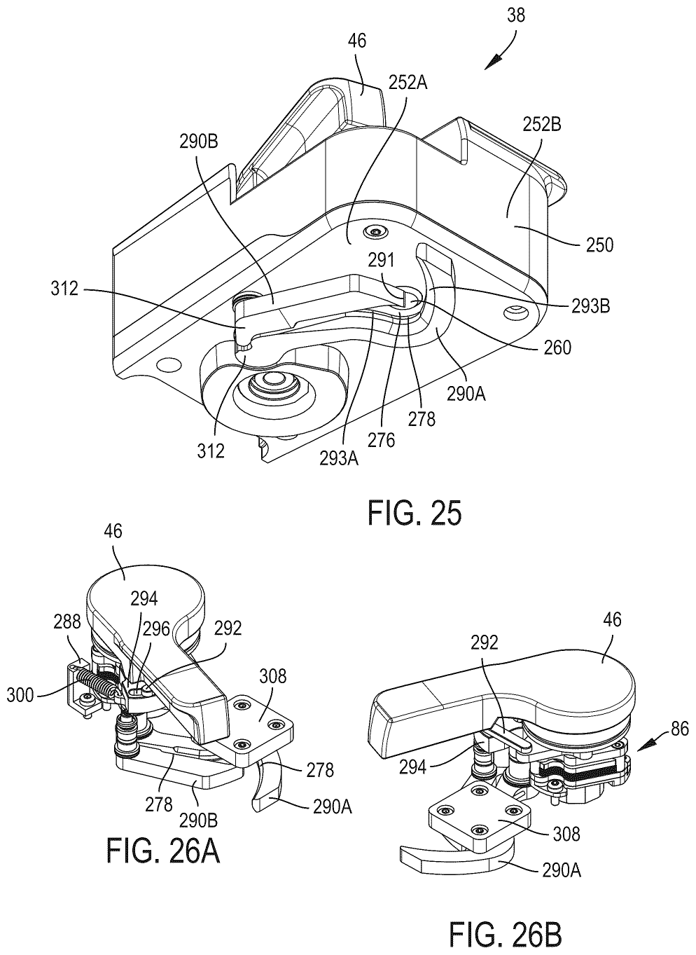

Referring now to FIGS. 25-27, various views of another exemplary embodiment of a drive head 38 are depicted. As shown, drive head 38 may include a housing 250. Example drive heads 38 may also include a first plunger flange grasper body 290A and a second plunger flange grasper body 290B. Each plunger flange grasper body 290A, B may be pivotally displaceable about respective pivot axes. In certain examples, the plunger flange grasper bodies 290A, B may be geared to one another. Pivotal displacement of the first plunger flange grasper body 290A in a first pivotal direction may engender pivotal displacement of the second plunger flange grasper body 290B in an opposite, second pivotal direction. Exemplary drive heads 38 may also include an actuator 46 which may be displaceable from a home position to an actuated position. The actuator 46 may be coupled to one of the plunger flange grasper bodies 290A, B via a transmission including a linkage 292 and a pivot body 294. The pivot body 294 may include a slot 296 in which a projection 298 extending from the linkage 292 may be disposed. Displacement of the actuator 46 from the home position to the actuated position may be transmitted through the transmission to the first plunger flange grasper body 290A. As the second plunger flange grasper body 290B is geared to the first plunger flange grasper body 290A, this may displace the plunger flange grasper bodies 290A, B to a spread apart, open state. In some examples, the plunger flange grasper bodies 290A, B may be biased toward a closed position by at least one bias member 300. The actuator 46 may be biased to the home position by at least one additional bias member 302. When the actuator 46 is released by a user, it may, under power of the additional bias member(s) 302, be driven back to the home position. In the event that a plunger flange 28 is present between the plunger flange grasper bodies 290A, B, the plunger flange grasper bodies 290A, B may be driven into the plunger flange 28 retaining it in place. This may inhibit the plunger flange grasper bodies 290A, B from fully returning to the closed position. The slot 296 in the pivot body 294 may allow for travel of the actuator 46 and attached linkage 292 independent of displacement of the plunger flange grasper bodies 290A, B when the plunger flange grasper bodies 290A, B are blocked from fully reaching the closed position. That is, the slot 296 may allow clearance for the projection 298 on the linkage 292 to displace absent corresponding displacement of the pivot body 294 as the actuator 46 is urged toward the home position by the at least one additional bias member 302.

The shapes of the first and second plunger flange graspers 290A, B may be selected to center plunger flanges 28 within an accepted size range (e.g. 1 cc-60 cc syringe) on a fixed fiducial reference axis. This axis may also be the same axis which a barrel grasper assembly 22 centers the bore of a syringe 18 upon. The axis may extend through the center of a contact 260 which mechanically communicates with and transmits applied forces to a load cell 330 (see, e.g., FIG. 32) of a sensing assembly 308 in the housing 250. Thus, a sensing assembly 308 may be included and utilized with relatively little complexity. In the example, the sensing assembly 308 would be coupled in stationary fashion to the drive head 38 housing via mounting screws 309. Additionally, the manner in which force is transmitted from the contact 260 to the sensing assembly 308 may be substantially the same across the range of accepted syringes 18. Thus, regardless of the syringe 18 type, adjustment of data from the sensing assembly 308 to account for variation in the way force is communicated via the contact 260 to the sensing assembly 308 may not be necessary. In alternative embodiments, any sensing assemblies 308 described herein may be used in place of that shown in FIGS. 25-27.

Still referring to FIGS. 25-27, in certain example embodiments, the housing 250 may include a first housing section 252A and a second housing section 252B. The first housing section 252A, may include an aperture 254 through which the drive head tube 36 is coupled to the drive head 38. The drive head 38 may include an actuator 46 (shown as a lever in the example embodiment). The second housing portion 252B may include a recess providing clearance for the actuator 46 to be displaced. The actuator 46 may be actuated to allow a user to freely displace the drive head 38. As further described elsewhere herein, the actuator 46 may, for example, disengage a clutch assembly 86 (see, e.g., FIGS. 41A-43B or alternatively disengage a nut assembly 140 (see, e.g., FIGS. 59A-60D) from a leadscrew 84.

Still referring to FIGS. 25-27, each plunger flange grasper body 290A, B is coupled to a pivot pin 310 which extends into the interior of the housing 250. Rotary seals may be provided to ensure the interior of the drive head 38 remains isolated from the surrounding environment. Such seals may also be included for actuators 46 described herein as well as plunger flange grasper bodies 256 of FIGS. 22A-24. As depicted in FIGS. 25-27, each of the plunger flange grasper bodies 290A, B may contact the other at a toothed region 312. The toothed region 312 of each plunger flange grasper body 290A, B may surround the plunger flange grasper body's 290A, B pivot axis. Thus the plunger flange grasper bodies 290A, B may be geared together and pivotal motion of one plunger flange grasper body 290A, B may generate a pivotal displacement of the other plunger flange grasper body 290A, B in the opposing direction. As best shown in FIG. 27, the pivot pin 310 coupled to the first plunger flange grasper body 290A may include a terminal end 314 disposed internal to the housing 250 which is keyed. The pivot body 294 may include a cooperatively keyed receptacle into which the keyed terminal end 314 of the pivot pin 310 is coupled. This may ensure that the plunger flange grasper body 290A and pivot body 294 rotationally displace in tandem.

The pivot body 294 may be coupled to a bias member 300. The bias member 300 may also be coupled to an anchor post 288 defined on or coupled to the housing 250. The bias member 300 may exert a force urging the plunger flange grasper bodies 290A, B and pivot body 294 coupled thereto to the closed position. When the plunger flange grasper bodies 290A, B are blocked from returning to the closed position, the pivot body 294 may also be blocked. When retaining a plunger flange 28, the bias member 282 may exert a force through the pivot body 294 which robustly holds the plunger flange grasper bodies 290A, B in place against the plunger flange 28. The slot 296 defined in the pivot body 294 may provide a clearance path which allows the actuator 46 and linkage 292 to return fully to the home position as the additional bias member 302 restores. Thus, any clutch assembly 86 (see, e.g., FIGS. 41A-43B) or nut assembly 140 (see, e.g., FIGS. 59A-60D) actuated by the actuator 46 may be re-engaged with a leadscrew 84 of the drive arrangement 82. As shown, the bias member 300 is an extension spring. Bias member 302 is a torsion spring coupled to the actuator 46 and to a stationary portion of the example clutch assembly 86.

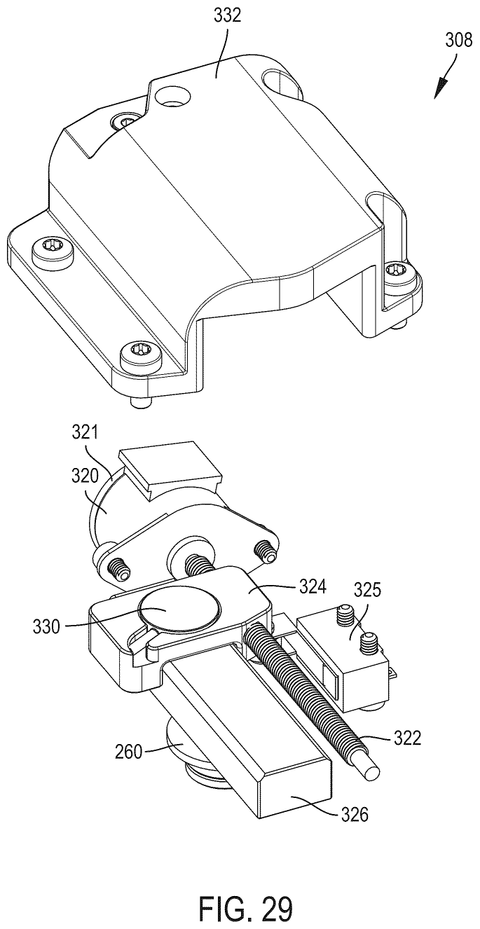

In the exemplary embodiment, the contact 260 (pin, plate, projection, button, or the like) extends through the housing 250 and is disposed in a plunger flange receiving region 276 on the housing 250. The contact 260 may be part of a sensing assembly 308 and may transfer force from an installed syringe 18 to a load cell 330 (see, e.g., FIG. 29) in the sensing assembly 308 such that this force may be monitored.

The plunger flange grasper bodies 290A, B may be shaped such that any plunger flange 28 in an accepted size range may be centered on a fiducial reference axis extending through the center of the contact 260 when the plunger flange grasper bodies 290A, B are closed on the plunger flange 28. One of the plunger flange grasper bodies 290B may be arranged to contact the plunger flange 28 at substantially a single point which may be located at a tip region 291 of the plunger flange grasper body 290B. The other of the plunger flange grasper bodies 290A, may be provided with two curved flange contacting surfaces 293A, B. One may have a convex curvature while the other may have a concave curvature. As shown, a curved transition region may separate the two flange contacting surfaces 293A, B. Each curved syringe contacting surface 293A, B of the plunger flange grasper body 290A may contact a retained plunger flange 28 substantially at one point. The curves of the flange contacting surfaces 293A, B may be derived such that the point along each flange contacting surface 293A, B that contacts the plunger flange 28 changes depending on the syringe 18 while keeping the center axis of the plunger flange 28 in a constant location independent on the type of syringe 18 being retained. In various examples, the contact points between the plunger flange grasper bodies 290A, B and the flange 28 may be approximately 120° apart from one another when a flange 28 is retained. The profile of the curved flange contacting surfaces 293A, B of plunger flange grasper body 290A may be dependent upon the range of syringe 18 diameters intended to be accepted by the syringe pump 10. The profile of the curved flange contacting surfaces 293A, B may also be dependent upon the gearing ratio between the plunger flange grasper bodies 290A, B.

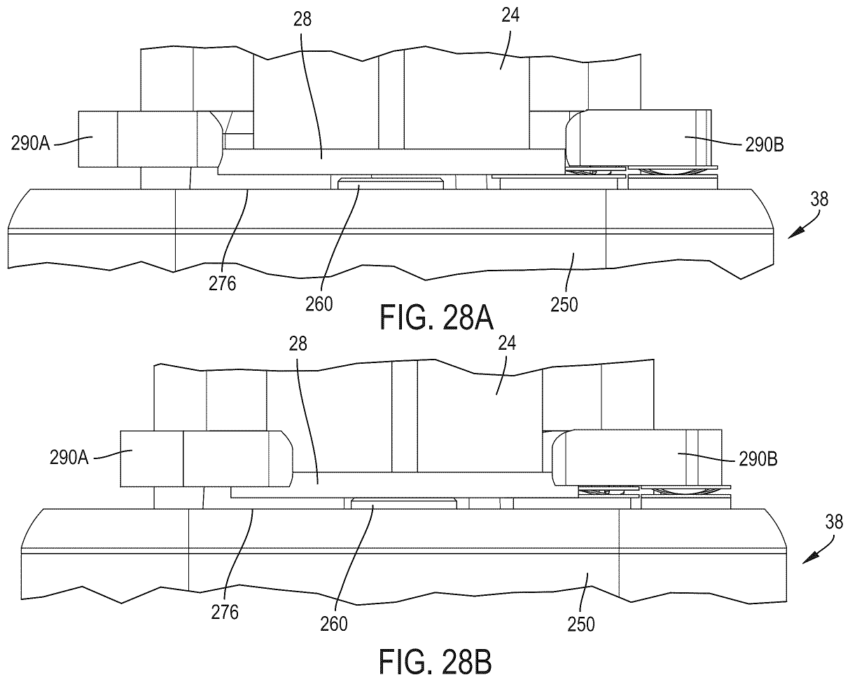

Referring now to FIGS. 22A-28B, in some embodiments, plunger flange grasper bodies 256, 290A, B may include a snugging face 278. Snugging faces 278 may be sloped or beveled. As best shown in FIGS. 28A-B, which depict views of the plunger flange grasper bodies 290A, B of the drive head 38 shown in FIGS. 25-27, as plunger flange grasper bodies 256, 290A, B are displaced to retain a plunger flange 28, the snugging faces 278 may make the initial contact with the plunger flange 28. As the plunger flange grasper bodies 256, 290A, B are driven into the plunger flange 28, the plunger flange 28 may be snugged or pulled up against the housing 250 and into physical communication with the contact 260 due to the contours of the snugging faces 278 (see, e.g., FIG. 28B). The snugging faces 278 may be angled and sized such that this occurs regardless of the syringe 18 being utilized. Example plunger flange grasper bodies 256, 290A, B may be constructed of a rigid material such as various metals or rigid plastic. This, may limit any tendency of the plunger flange grasper bodies 256, 290A, B to bend helping to ensure the plunger flange 28 is sturdily held in place against the contact 260.

The example drive heads 38 described above in relation to FIGS. 22A-28B may be particularly desirable as standard plunger flanges 28, regardless of size or thickness, may be centered on and pulled into an abutting relationship with the contact 260 when the plunger flange grasper bodies 256, 290A, B are closed upon the plunger flange 28. This may ensure that regardless of the syringe 18 being used, force is applied to the sensing assembly 308 in the same manner. Since the plunger flange 28 may be centered on a fiducial reference axis, the sensing assembly 308 may be fixedly coupled to the housing 250 in a stationary manner. By centering the plunger flange 28, data from a load cell monitoring pressure in the syringe 18 may be used to make robust determinations related to pressure inside the syringe 18. The plunger 24 of the syringe 18 may also be supported against bending out of alignment with the long axis of the syringe barrel 20. This may increase delivery accuracy as well as decrease the complexity while increasing the accuracy of pressure estimates generated based on data collected by a load cell 330 (see, e.g. FIG. 32). Furthermore, when the plunger 24 is properly centered, there may be less and more consistent friction between the syringe barrel 20 and the plunger 24. Additionally, as the plunger 24 is driven to and held in a centered location by the plunger flange grasper bodies 256, 290A, B, there may be less variability in plunger 24 position after loading of a new syringe 18. Such an arrangements may also allow for easier adoption of new syringe 18 types as less testing may be required when attempting to support additional syringes 18. Moreover, as the plunger flange 28 is pulled against the contact 260, the drive head 38 may prevent siphoning from the syringe 18. Again, this may be independent of the syringe 18 type or size. Furthermore, the sensing assembly 308 may be inhibited from losing physical communication with the plunger flange 28 during scenarios where negative pressure may be present in the syringe 18. Thus, the sensing assembly 308 may more reliably monitor pressure in the syringe 18 and may even detect negative pressure scenarios.

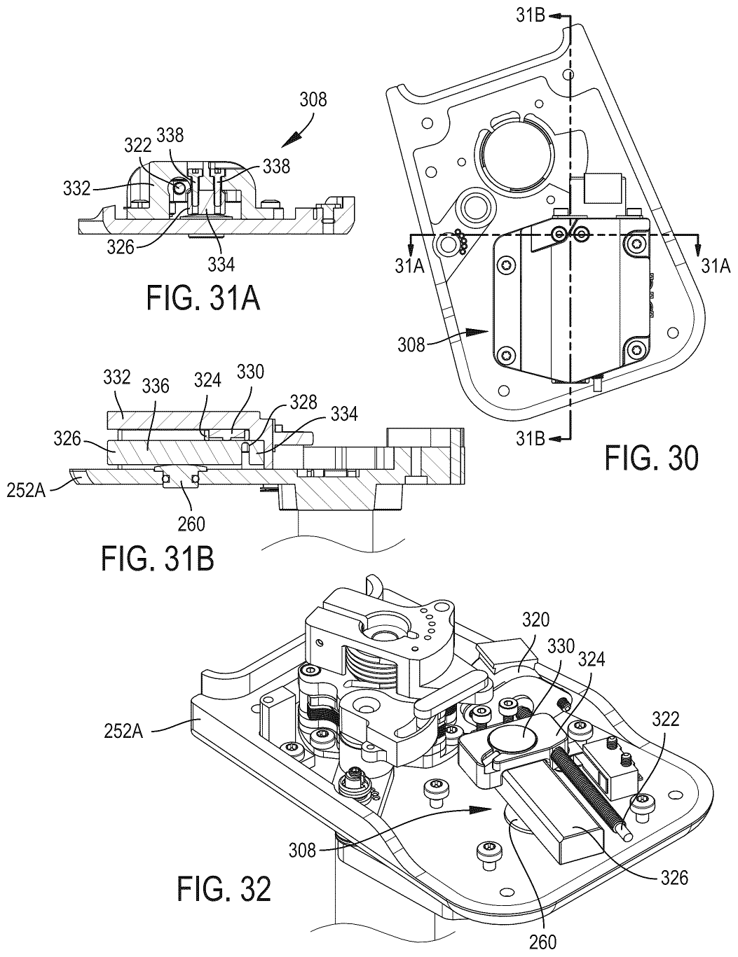

Referring now to FIGS. 29-32, an example sensing assembly 308 which may be included in any of the example syringe pumps 10 described herein is depicted. FIGS. 31A-B depict cross-sectional views of the sensing assembly 308 taken at the indicated cut planes of FIG. 30. The exemplary sensing assembly 308 may monitor force exerted by a syringe 18 against a contact 260 coupled to the drive head 38. Force data from the sensing assembly 308 may be used to estimate pressure in the syringe 18 as fluid is dispensed from the syringe 18 by the syringe pump 10. In example embodiments, the sensing assembly 308 may include a displaceable load cell 330. As shown in FIGS. 29-30, the sensing assembly 308 may include a load cell motor 320. The load cell motor 320 may be a stepper motor. The load cell motor 320 may be powered to drive rotation of a load cell leadscrew 322 coupled to the output of the load cell motor 320. The load cell 330 may be mounted to a load cell carriage 324 which may include a threaded bore which is in engagement with the load cell leadscrew 322. The sensing assembly 308 may also include a beam 326 having a flexure 328 (best shown in the cross section depicted in FIG. 31B). The beam 326 may be coupled to a cover 332 at a first portion 334 of the beam 326 on a first side of the flexure 328. In the example embodiment, a set of fasteners 338 (see, e.g., FIG. 31A) are used, though any suitable coupling may be used in alternative embodiments. The load cell carriage 324 may travel along a second portion 336 of the beam 326 on the opposing or second side of the flexure 328. The beam 326 may act as a guide for the load cell carriage 324.

Powering of the load cell motor 320 may be governed by a controller 15 of the syringe pump 10 to drive the carriage 324 and load cell 330 coupled thereto along the beam 326. A sensor 321 (e.g. motor encoder, linear encoder, potentiometer, or other linear position sensor such as those described herein) may be monitored to determine the linear position of the load cell 330 along the beam 326. In certain examples, a home switch 325 (e.g. microswitch) may be included. A controller 15 of the syringe pump 10 may govern powering of the load cell motor 320 based at least in part on data received from the sensor 321 and optionally the home switch 325 to reach a target position for the carriage 324.

The range of forces measured by the load cell 330 may be function of the area of the plunger 24 piston within the syringe 18. Where, for example, a syringe pump 18 is intended to support use of 1-60 ml syringes, the range of forces reasonably expected to be measured by the load cell 300 may be quite large (e.g. 0.2N for 100 mmHg pressure in a 1 ml syringe to greater than 90N for a 1000 mmHg in a 60 ml syringe). A single load cell 330 with a defined capacity may be used and the ratio of applied force to the load cell 330 may be varied by adjustment of the carriage 324 position along the beam 326. This may allow for forces in excess of the capacity of the load cell 330 to be applied via the syringe 18 to the sensing assembly 308 without damage to the selected load cell 330. It may also allow for adjustments of the sensitivity of the sensing assembly 308 given a constant syringe 18 type.

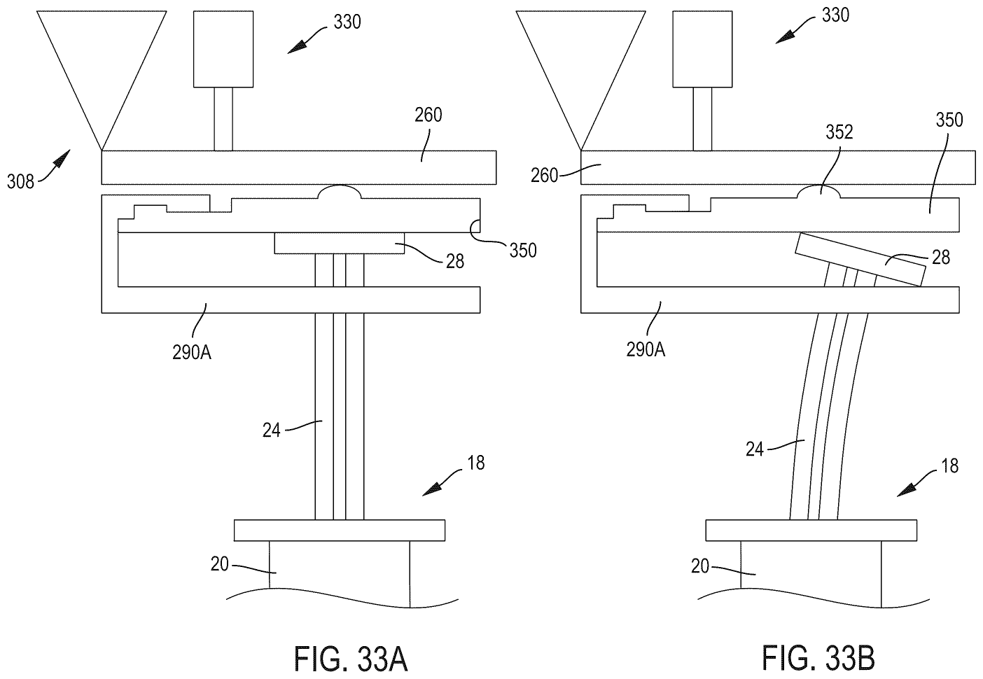

The face of the beam 326 opposite the load cell 330 may be in abutment with a contact 260 in the plunger flange receiving area 276 of the drive head 38. The contact 260 may be sealed to the housing 250 of the drive head 38 with an o-ring or other gasket. In the example embodiment, the contact 260 is in abutment with the central portion of the second side of the beam 326. Force on the plunger 24 of an installed syringe 18 may be transmitted through the contact and beam 326 to the load cell 330. In certain embodiments, such an arrangement may be used in conjunction with a force concentrator 350 (see, e.g., FIGS. 33A-35).

Given that the type of syringe 18 installed on a syringe pump 10 is known (e.g. from sensing the position of portions of a barrel grasper 22), an optimum or target position of the load cell carriage 324 may be determined. This may be accomplished by the controller referencing the syringe 18 identity against a look-up table in certain examples. The optimum or target position may be a position which provides the best possible resolution from the load cell 330 while at the same time not risking overloading the load cell 330. A controller 15 of the syringe pump 10 may govern operation of the load cell motor 320 to drive the carriage 324 to this position based at least in part on feedback from the sensor 321. This may be done at any point after the type of syringe installed 18 on the syringe pump 10 is determined. As mentioned elsewhere herein, example drive heads 38 may include an actuator 46 which may be displaced to allow translational displacement of the drive head 38 (e.g. when installing a new syringe 18). In some examples, displacement of the carriage 326 may begin or partially occur (e.g. start before) while a user is displacing the drive head 38 a desired position. Displacement of the carriage 326 may continue (or begin to be commanded by a controller 15) after a plunger flange 28 is retained by plunger flange graspers 256, 290A, B of the drive head 38.

In embodiments where a drive head 38 includes plunger flange graspers 256, 290A, B with snugging faces 278, as the plunger flange graspers 256, 290A, B are displaced to retain the plunger flange 28, the snugging faces 278 may push the plunger flange 28 against the contact 260. This will create an axial load pressing the contact against the beam 326. Before the syringe pump 10 proceeds to deliver from the syringe 18, the load cell 330 may be tared or zeroed in any examples where snugging faces 278 are provided. In other examples, an offset may be recorded and applied by the controller 15 to future readings received from the sensing assembly 308. Where snugging faces 278 are included with a motorized sensing assembly 308, the load cell motor 320 may be sufficient to drive the carriage 326 even with the presence of the axial load. As the plunger flange graspers 256, 290A, B apply an axial load, it may be possible to monitor for negative pressure scenarios which may develop in the syringe 18. Should the pre-load be observed to decrease, it may be inferred that negative pressure has developed in the syringe 18.

Referring now to FIGS. 33A-35, in certain example syringe pumps 18, a barrel 20 of an installed syringe 18 may be held in place (e.g. via a barrel clamp assembly 484 or other retainer) against a recess in a body or surface of the syringe pump 10 (e.g. front bezel 13A). The recess may be formed by two surfaces disposed at an angle to one another. Thus, the recess may be in the shape of the Latin character “V” in certain examples. Depending on the diameter of the barrel 20, the axis of the syringe 18 may be at a different position with relation to the recess. Smaller syringes 18, for example, will sit deeper in the “V” shaped recess. As a result, the position of the axis of the syringe 18 with respect to the contact 260 on the drive head 38 may differ depending upon the diameter of the barrel 20 of the syringe 18. An exemplary syringe retention assembly 19 of this type is described in greater detail in relation to FIGS. 16A-21B.

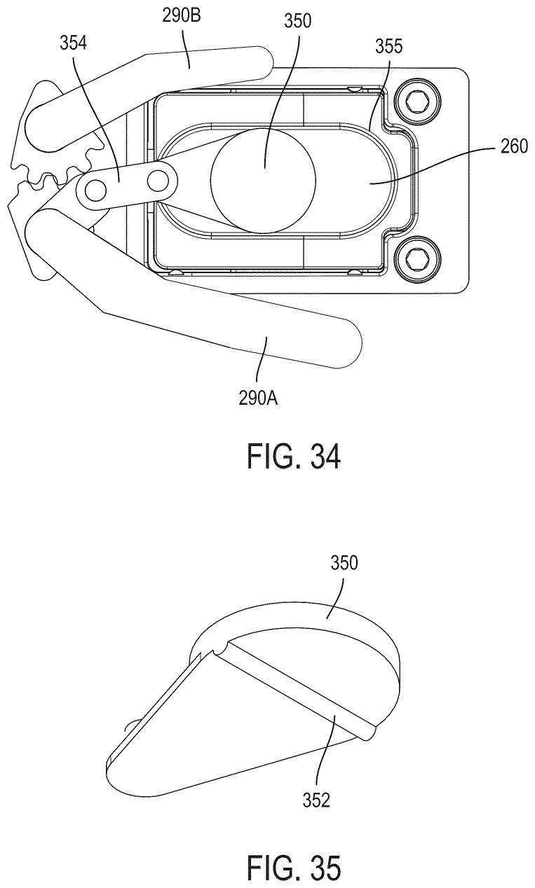

An example block diagram of a portion of a drive head 38 is depicted in FIGS. 33A-B. In some such embodiments, plunger flange grasper bodies 290A, B of the drive head 38 may be paired with a force concentrator 350. An example set of plunger flange graspers 290A, B with a force concentrator 350 and a force concentrator 350 depicted in isolation are shown in FIGS. 34-35 respectively. Though the force concentrator 350 is depicted as generally planar, other shapes are also possible. The force concentrator 350 may be coupled to the plunger flange grasper bodies 290A, B with a linkage 354 which engenders translational displacement of the concentrator plate 350 as the plunger flange grasper bodies 290A, B rotationally displace. A guide 355 may be included to assist in ensuring that the force concentrator 350 is constrained to displace substantially along a defined axis. As best depicted by FIGS. 33A-B, the contact 260 included in the drive head 38 may be a pivoting plate. When force is applied to the syringe 18 plunger 24 through the drive head 38, the force may be transmitted through the concentrator plate 350 to the plunger 24 (which may be in abutting relationship with the concentrator plate 350). The concentrator plate 350 may include a concentrator body 352. The concentrator body 352 may be a protuberance projecting from at least a portion of a face of the force concentrator 350 closest to the contact 260. In the example, the concentrator body 352 is formed by a raised rib which extends across a face of the force concentrator 350. The concentrator body 352 may physically touch the contact 260 and all force against the plunger 24 may be concentrated at the region or point where the concentrator body 352 and contact 260 touch.

As the contact 260 is pivotally coupled to the drive head 38, the contact 260 may act as a lever which increases or decreases the force on the load cell 330 of a sensing assembly 308 depending on where the force is applied. The force may be increased or decreased by some magnitude depending at least in part on the region of the contact 260 which the concentrator body 352 is touching. Given that the type of syringe 18 installed in the syringe pump 10 is known (this may be determined as described elsewhere herein), an estimated position of the plunger flange graspers 290A, B when the plunger flange 28 is in a grasped state can also be known. In turn, the position of the concentrator plate 350 and region of the contact 260 touched by the concentrator body 352 may be estimated. This may allow for data collected by the load cell 330 to be analyzed for any increase or decrease in force applied to the load cell 330 due to the position of the concentrator body 352 along the lever contact 260. For example, the controller 15 may automatically apply an adjustment to the data based on the diameter of the plunger flange 24 of the installed syringe 18.

Inclusion of a concentrator plate 350 may ensure that adjusted data from the load cell 330 is more reliable. As shown in FIG. 33B, in certain scenarios, the plunger 24 of a syringe 18 may bend off axis. Such bending is shown in exaggerated manner for illustrative purposes in FIG. 33B and typical bending may generally be more subtle. When bending is present, it may occur during loading of the syringe 18 onto the syringe pump 10. Typically, this bending may be challenging to recognize as the bending may occur in a direction toward the user loading the syringe pump 10. Such distortion of the plunger 24 may result in the touch point of the plunger flange 28 along the concentrator plate 350 differing from what would be expected based on the syringe 18 being used. Additionally, some plunger flanges 28 may include raised gripping assistance features which may not contact the concentrator plate 350 in a predictable location. Since the concentrator plate 350 includes a concentrator body 352 which is stationary with respect to the remainder of the concentrator plate 350, the region of the contact 260 to which force is applied may stay substantially constant regardless of whether the plunger 24 is straight, bowed/bent off axis, or includes raised features in an uncontrolled position. Thus, an adjustment of load cell 330 data based on the syringe 18 being used may be more accurate when force is concentrated with a force concentrator 350 such as that shown. Absent such a concentrator plate 350, the plunger flange 28 of an installed syringe would directly touch the contact 260. Bowing or bending of the plunger 24 would, for example, alter the point of contact between the plunger flange 28 and contact 260 from what would be expected for a given syringe 18. Thus, adjustment of data from the load cell 330 due to the lever contact 260 may be difficult to perform accurately.

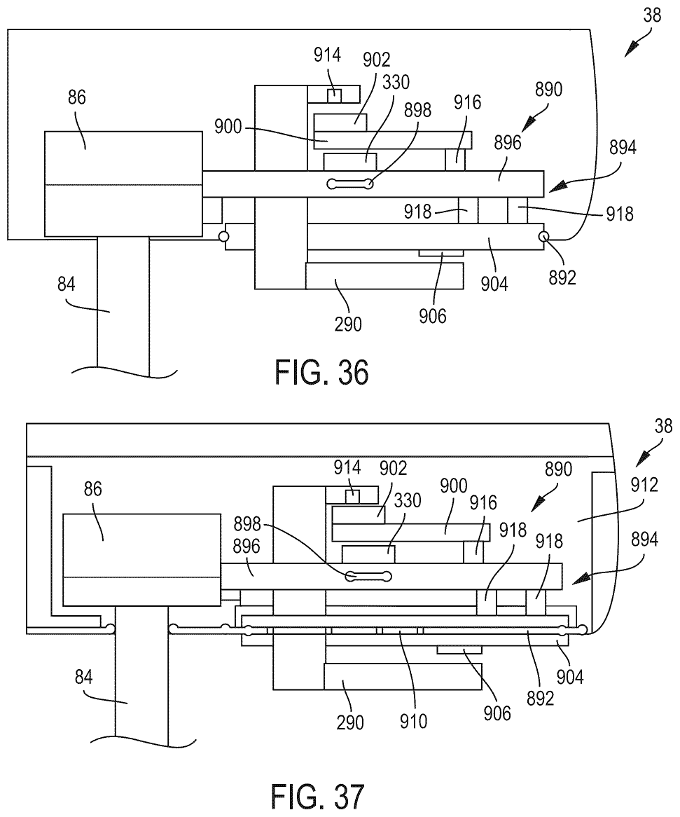

Referring now to FIGS. 36-37, in certain embodiments, drive heads 38 may include a floating assembly or portion 890 which may be displaceable as a unit within the drive head 38. Two block diagrams of exemplary drive heads 38 including floating assemblies 890 are depicted in FIGS. 36-37. Example floating assemblies 890 may be displaceable relative to the housing 250 of the drive head 38. A sealing member 892 may be present at any interface between the floating assembly 890 and the housing 250 of the drive head 38. The scaling member 892 may maintain isolation of the interior and exterior of the housing 250 as the floating assembly 890 displaces with respect to the housing 250. In certain examples, the sealing member 892 may also act as a diaphragm which may help to facilitate displacement of the floating assembly 890 with respect to the housing 250.

A floating assembly 890 may include a load cell assembly 894. The load cell assembly 894 may be coupled to the housing 250. In some examples, the load cell assembly 894 may be directly anchored to a portion of the housing 250 or alternatively to portion of the drive head 38 which is fixed relative to the housing 250 (e.g. via one or more fastener). In the examples shown, the load cell assembly 894 is cantilevered from a portion of the end of the drive head tube 36 disposed on the interior of the housing 250. All other components of the floating portion 890 of the drive head 38 may be coupled to the load cell assembly 894.

Various example load cell assemblies 894 may include a beam 896 with a flexure 898. A load cell 330 may be disposed on the beam 896 so as to monitor deflection of the beam 896 at the flexure 898. A sensor circuit board 900 in electrical communication with the load cell 330 may be coupled to an unsupported end of the beam 896. The sensor circuit board 900 may also include one or more additional sensors. In the example, a magnetic sensor 902 for monitoring the position of a plunger flange grasper 290 is included on the sensor circuit board 900. The magnetic sensor 902 may generate an output signal which varies in relation to the position of a magnetic or metal body 914 attached to the plunger flange grasper 290. One or more fastener 916 may be used to couple the sensor circuit board 900 to the beam 896. The sensor circuit board 900 may additionally or instead be fastened to other components of the floating assembly 890 (e.g. one or more of the panels 904, 908 described in the following paragraphs). The sensor circuit board 900 may be coupled to a flex cable which may be routed into a housing 12 of a syringe pump 10 through the drive head tube 36.

The floating assembly 890 may also include a panel 904. The panel 904 may have a face which is accessible from the exterior of the drive head 38. The panel 904 may include a raised feature such as a nub or plateau 906 on the exteriorly accessible face. A plunger flange 28 of a syringe 18 may seat against the plateau 906 when retained by plunger flange graspers 290 of the drive head 38.

In certain examples such as that shown in FIGS. 37, the panel 904 may be paired with a second panel 908 disposed on the interior of the drive head 38. Panel 904 and second panel 908 may be coupled to one another via one or more fastener 910. One or both of the panels 904, 908 may be fastened to the unsupported end of the beam 896 (e.g. by one or more fastener 918). A portion of the sealing member 892 may be captured and compressed between the panels 904, 908. As shown in FIG. 37, the sealing member 892 may span a gap between the periphery of the panels 904, 908 and the surrounding portions of the housing 250. Thus, the scaling member 892 may act as a diaphragm which supports displacement of the floating assembly 890 relative to the housing 250. The drive head 38 may also include a tray 912 which may be coupled to the housing 250. Portions of the sealing member 892 may be captured and compressed between the tray 912 and the interior faces of the housing 250 to form a substantially fluid tight seal isolating the interior volume of the housing 250 from the surrounding environment. The panels 904, 908 may be constructed of a stiff material (e.g. metal such as aluminum or steel).

Though only one plunger flange grasper 290 is depicted for sake of illustration in each of FIGS. 36-37, it should be understood that a plurality of plunger flange graspers would typically be included and any of those described herein may be used in a drive head 38 with a floating portion 890 (see, e.g., plunger flange graspers 256, 290A, B of FIG. 22A and FIG. 27). The plunger flange graspers 290 of the drive head 38 may be included as part of the floating portion 890 of the drive head 38. Plunger flange graspers 290 may be coupled to a portion of the floating assembly 890 and be rotatable relative to the remainder of the floating assembly 890. For example, each of the plunger flange graspers 290 may be rotatably mounted to one (or both) of the panels 904, 908. Additionally, as the sensor circuit board 900 and plunger flange graspers 290 are each included in the floating assembly 890, the distance between the magnetic sensor 902 and the metallic body 914 on one of the plunger flange graspers 290 may be held substantially constant regardless of any displacement of the floating assembly 890.

As the plunger flange graspers 290 are part of the floating assembly 890, the plunger flange graspers 290 may press the plunger flange 28 against the nub 906 on panel 904 when in a grasping state against the plunger flange 28 without establishing a preload perceived by the load cell 330. This may simplify measurement of forces due to pressure conditions in an installed syringe 18 during operation of a syringe pump 10 and facilitate inclusion of snugging face 278 on the plunger flange graspers 290 (sec. e.g. FIGS. 28A-B). Zeroing of the load cell 330 data may not be needed. Such an arrangement may also limit syringe to syringe variability. Additionally, by retaining the plunger flange 28 against a floating portion 890 of the syringe pump 10, negative pressure conditions in a syringe 18 may be sensed. Moreover, a transition between a positive pressure condition and a negative pressure condition in a syringe 18 may be clearly perceived via data output from the load cell 330.

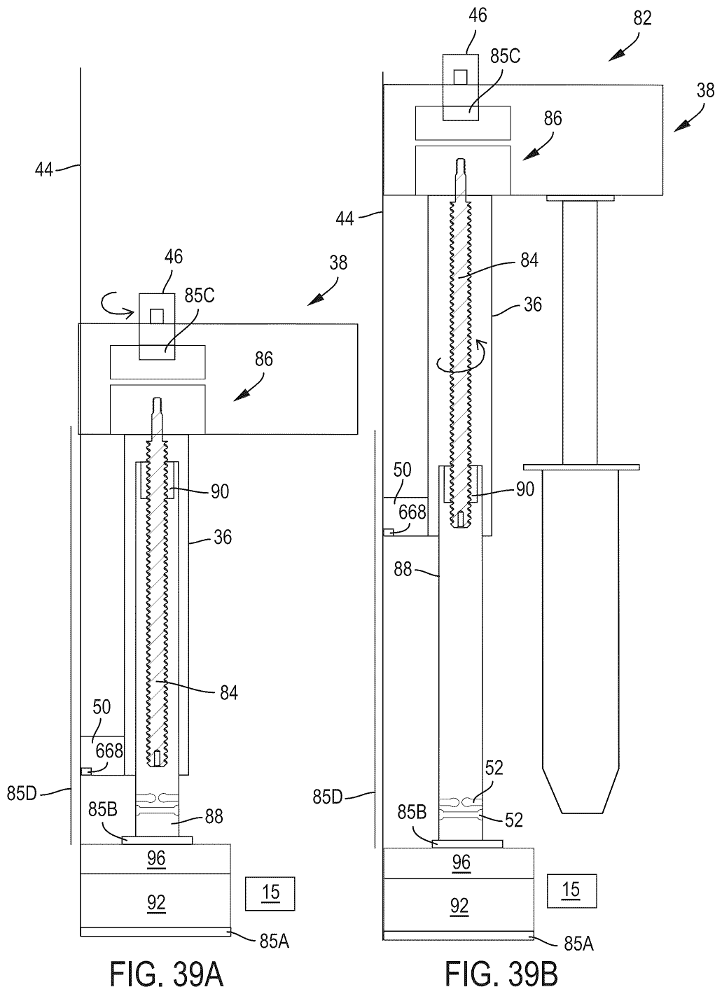

Referring now to FIGS. 38A-39B, exemplary block diagrams of an example drive assembly 82 are depicted. The example drive assembly 82 may include a first portion 80A and a second portion 80B. The first portion 80A may be a driven portion of the drive assembly 82. The second portion 80B may be a driving portion of the drive assembly 82 which may be operated to engender displacement of the first portion 80A of the drive assembly 82. The first portion 80A may translationally displace with respect to the second portion 80B. A bearing surface 44 may guide and support the first portion 80A as translational displacement of the first portion 80A transpires. In some examples, the bearing surface 44 may be provided upon a rigid backbone of the syringe pump 10 to which the handle 34 is coupled or in which the handle 34 is formed (further described in relation to FIGS. 64A-C).

As shown, the first portion 80A of the drive assembly 82 may include a leadscrew 84, a drive head tube 36, a clutch assembly 86, and a drive head 38. The leadscrew 84 may be disposed within the drive head tube 36 and may be coupled to a clutch assembly 86 disposed within the drive head 38. The clutch assembly 86 may be any of those shown or described herein (see, e.g., FIGS. 41A-43B. The clutch assembly 86 may be transitioned between an engaged state (FIGS. 38A-B) and a disengaged state (FIGS. 39A-B) by displacement of an actuator 46 (e.g. a lever body such as that shown in FIGS. 1-3). With the clutch assembly 86 in the engaged state, the leadscrew 84 may be prohibited from rotation within the drive assembly 82. The leadscrew 84 may be free to rotate about its axis of elongation when the clutch assembly 86 is in the disengaged state.

The second portion 80B of the drive assembly 82 may include a nut tube 88 having a nut 90 coupled thereto. A drive motor 92 and transmission 96 may also form part of the second portion 80B. As shown, the nut 90 may be coupled within the nut tube 88 (e.g. at an end thereof) and have threading which cooperates with and engages that of the leadscrew 84. The nut 90 may be fixed with respect to the nut tube 88 and may be substantially incapable of axial or rotational displacement relative to the nut tube 88. The nut 90 may be disposed concentric with the axis of the nut tube 88 and may be formed as a monolithic body. The nut 90 may be in a permanently engaged relationship with the leadscrew 84 and the nut 90 may not be disengagable from the leadscrew 84. While the nut 90 may be referred to as permanently engaged with the leadscrew 84 and not disengagable with the leadscrew 84, these terms are not intended to encompass scenarios where the drive assembly 82 is disassembled, destroyed, etc.

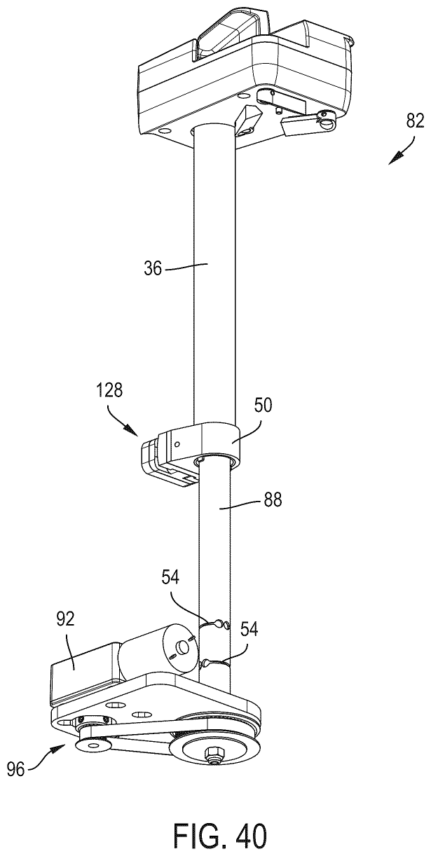

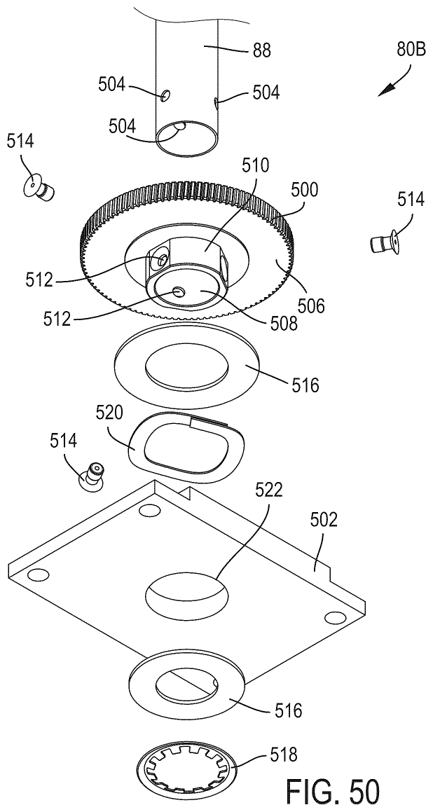

The nut tube 88 may be coupled to a drive motor 92 via a suitable transmission 96 (e.g. via one or more gears, belts, etc.). An example with a belt driven transmission 96 is shown in FIG. 40. The coupling between the nut tube 88 and the drive motor 92 may be a non-back-driveable coupling (e.g. worm drive or high gearing ratio). In certain examples, the drive motor 92 may rotate a worm gear which interdigitates with a worm wheel coupled to the nut tube 88. Such a worm wheel may be coupled to an end region of the nut tube 88 opposite the nut 90 (see, e.g., FIGS. 50-58C). A controller 15 of the syringe pump 10 may govern operation of the drive motor 92 in order to generate rotation of the nut tube 88 and nut 90 about the axis of the nut tube 88.

Still referring to FIGS. 38A-39B, with the clutch assembly 86 in the engaged state, as the nut tube 88 is rotated by the drive motor 92, the leadscrew 84 may be displaced into or out of the nut tube 88 (see, e.g., FIGS. 38A-B). Thus the driven portion 80A of the drive assembly 82 may be displaced translationally with respect to the driving portion 80B of the drive assembly 82. The drive head tube 36 may telescope over and away from the nut tube 88 as the driven portion 80A is displaced. As the driven portion 80A of the drive assembly 82 is displaced over the nut tube 88, the drive head 38 of the syringe pump 10 may urge fluid out of an installed syringe 18. The plunger 24 of a syringe 18 installed on the syringe pump 10 may be driven into a barrel 20 of that syringe 18 as the drive head 38 is displaced. The drive motor 92 may be powered as needed to dispense fluid from the syringe 18 until fluid in the syringe 18 is depleted or it is desired to cease dispensing of fluid.

The drive head 38 may include a bearing arrangement which rides along a bearing surface 44 defined on a rigid backbone of the syringe pump 10 as the driven portion 80A of the drive assembly 82 is displaced. Additionally, the drive head tube 36 may include a carriage 50 coupled to an end of the drive head tube 36 opposite the drive head 38. The carriage 50 may also be guided and supported by the bearing surface 44 as the driven portion 80A of the drive assembly 82 is displaced. Thus, the driven portion 80A may be supported at bearing locations at its respective end regions. There may be at least one bearing point at each of the end regions of the driven portion 80A. In certain examples, the drive head 38 may have at least two contact points with the bearing surface 44 and the carriage 50 may have at least two contact points with the bearing surface 44 (e.g. each may have three bearing points in certain examples). The bearing points additionally may remain evenly spaced regardless of the translational position of the driven portion 80A along its displacement range. Thus, example drive assemblies 82 may have substantial stiffness which is uniform throughout the displacement range of the driven portion 80A.

This uniform stiffness may help to bestow a number of benefits. For example, less time may be needed to begin an infusion as components of the syringe pump 10 may resist distortion under forces resulting from applying pressure to the plunger 24 of an installed syringe 18. As distortion may be minimized, this may additionally help to increase the correlation between data from sensors 85A-D and the amount of volume dispensed from the syringe 18. Any eccentricities in the leadscrew 84 may have less of an effect on the position of the drive head 38. Moreover, due to the high level of stiffness in the drive assembly 82, backpressure events may result in minimal impact on flow rate from the syringe pump 18 when they are relieved. In low stiffness drive assemblies, backpressure may cause components to deflect and a bolus of fluid from the syringe 18 may result when backpressure is relived and these components restore to a less stressed state. Similarly, the potential for fluid to be delivered from the syringe 18 due to siphoning 18 may be mitigated by a highly stiff drive assembly 82 such as that shown in FIGS. 38A-39B. A highly stiff drive assembly 82 such as that shown in FIGS. 38A-39B may also facilitate more rapid detection of certain alarm conditions. For example, since the drive assembly 82 is highly stiff, absorption of the force resulting from pressure in the syringe 18 by components of the drive assembly 82 may be minimized. Thus, an occlusion downstream of the syringe 18 may be more rapidly detected by a sensor monitoring pressure in the syringe 18 (e.g. sensing assembly 308 of FIG. 27).

Still referring primarily to FIGS. 38A-39B, the nut tube 88 may include a number of sets of slots 54 which may be angularly offset from one another. An embodiment including such slots is also depicted in FIG. 40. In various examples, each set of slots 54 may be cut (e.g. laser cut) into opposing sides of the nut tube 88. In the example shown, two sets of slots 54 are included and each set is angularly offset by 90° from the other. The terminal ends of each slot 54 may be rounded and enlarged with respect to the mid-section of each slot 54. In the event that the leadscrew 84 includes any eccentricities, the slots 54 may allow the nut tube 88 to help absorb any resulting wobble or drunkenness of the nut 90 when the nut 90 is interfacing with the eccentric leadscrew 84 regions. Thus, the drive head 38 may be inhibited from being pushed about due to eccentricities in the leadscrew 84. This may increase the uniformity of flow rate from the syringe 18 over a given segment of time as fluid is dispensed from the syringe 18.

Example drive assemblies 82 may include one or more sensor 85A-D which may output respective signals that may be used to monitor displacement of the driven portion 80A of the drive assembly 82. There may, for example, be a sensor 85A monitoring the drive motor 92 (e.g. rotary encoder). There may be a sensor 85B monitoring the rotational position of the nut tube 88 (e.g. magnetic sensor, encoder, etc.). There may also be a sensor 85C monitoring the rotational position of the leadscrew 84. This sensor 85C may be monitored to ensure the clutch assembly 86 does not slip and may be used to determine the amount of leadscrew 84 rotation in the event that slippage occurs. In some embodiments, the carriage 50 position may be tracked by a sensor 85D such as a linear potentiometer. In alternative embodiments, sensor 85D may be a linear position sensing assembly 650 such as one of those shown and described in relation to FIGS. 65A-73H. In such examples a target assembly 668 may be included on the carriage 50. The controller 15 may, in certain examples, compare data from sensor 85D to data from the motor encoder 85A to monitor for clutch slippage.

A controller 15 of the syringe pump 10 may analyze the output from the one or more sensors 85A-D to determine and track displacement of the drive head 38 and thus the amount of fluid dispensed from a syringe 18 installed on the syringe pump 10. The controller 15 may also generate commands governing displacement of the driven portion 80A based at least in part on signals generated by the one or more sensors 85A-D. In some embodiments, data from certain sensors (e.g. sensor 85D and sensor 85A) may be compared to determine an amount of backlash in the drive assembly 82. Data from certain sensors (e.g. sensor 85D and sensor 85A) may be compared to determine the stiffness of the drive assembly 82. In certain examples, stiffness and or backlash determinations may be checked against historic or previous determinations (or a predefined threshold) to monitor wear in the drive assembly 82. In the event that wear exceeds or is approaching a predefined limit, the controller 15 may generate a notification or alert that the syringe pump 10 should be scheduled for service.

With the clutch assembly 86 in the disengaged state, the leadscrew 84 may be free to rotate. As mentioned, the drive motor 92 may be prevented from being back-driven. A user may displace the drive head 38 in a direction parallel to the leadscrew 84. The leadscrew 84 may rotate so as to allow the leadscrew 84 to translationally displace with respect to the nut 90 (see, e.g., FIGS. 39A-B). The leadscrew 84 may have multi-start, high pitch threading to facilitate rapid manual displacement of the driven portion 80A when the clutch assembly 86 is disengaged. Disengagement of the clutch assembly 86 and displacement of the drive head 38 may allow for a used or depleted syringe 18 to be removed from the syringe pump 10. It may also facilitate installation of a new, filled syringe 18 on the syringe pump 10. Once a new syringe 18 is installed on a syringe pump 10, the drive head 38 may be displaced toward the plunger flange 28 of that syringe 18. With the drive head 38 in place, the actuator 46 may be displaced to re-engage the clutch assembly 86. The controller 15 may command operation of the drive assembly 82 to begin dispensing of fluid from the newly installed syringe 18.

The example drive assembly 82 may be advantageous for a variety of reasons in addition to those listed above. The drive assembly 82 may be substantially anti-backlash and be operated with minimal free-rotation of the nut 90 before displacement of the leadscrew 84 (and components coupled thereto) occurs. As the nut 90 is permanently in engagement with the leadscrew 84, there would be no need to ensure that threads of the leadscrew 84 and nut 90 are appropriately aligned before powering the drive motor 92 to dispense fluid from an installed syringe 18. This may reduce the time needed to begin an infusion once a syringe 18 is installed on the syringe pump 10. It may also obviate the need for a locating sequence where the drive motor 92 is briefly powered in a forward and reverse direction to ensure that the threads of a previously disengaged half-nut and leadscrew 84 are suitably engaged. The example drive assembly 82 depicted in FIGS. 38A-39B may further be advantageous as it is particularly compact and uses only a small number of components. As the nut 90 is permanently engaged with the leadscrew 84, no space is needed for portions of the nut 90 to displace out of engagement with the leadscrew 84 thread. Additionally components for actuating movement of portions of the nut 90 relative to leadscrew 84 are not included. Due to the more compact design, space may be more readily available to use larger and stiffer nuts 90 and leadscrews 84 in the drive assembly 82 further increasing stiffness of the drive assembly 82. As the nut 90 may be placed at least partially within the nut tube 88, the nut 90 may be disposed coaxially with respect to the nut tube 88 and drive head tube 36. This may limit offset loading as the drive assembly 82 is operated, thus a more direct load path may be present and less strain on the drive train may be generated.

Referring now to FIGS. 41A-B, an exploded view and a cross-sectional view exemplary embodiment of a clutch assembly 86 are respectively depicted. An actuator 46 for the clutch assembly 86 is also depicted. The actuator 46 is shown as a lever type actuator. The clutch assembly 86 may include a clamp 810 which may seat within a recess of the actuator 46. The clamp 810 may be coupled (e.g. via one or more fastener such as a bolt) to the actuator 46 and rotate in tandem with the actuator 46. The clamp 810 may include an aperture 814 which may accept a stem 816 of a drive screw 818 of the clutch assembly 86. The clamp 810 includes two cantilevered bodies 812A, B through which a screw 820 extends. With the stem 816 of the drive screw 818 positioned within the aperture 814, the screw 820 may be tightened to pinch the aperture 814 closed against the stem 816. This may inhibit relative motion between the clamp 810 and the drive screw 818. Thus, the drive screw 818 may rotate as a user displaces the actuator 46.

The clutch assembly 86 may also include a set of end plates 822A, 822B. A first of the end plates 822A may be disposed most proximal the actuator 46 and may include a threaded aperture 824. When the clutch assembly 86 is assembled, a threaded region 826 of the stem 816 of the drive screw 818 may be engaged with the threaded aperture 824 in the first end plate 822A. The second end plate 822B may be coupled (e.g. by fasteners 825) to a portion of the drive head housing 250 (see, e.g., 252A of FIG. 25) rendering the second end plate 822B stationary with respect to the drive head housing 250. The end plates 822A, B may be coupled to one another via a set of fasteners 828 (e.g. bolts). The fasteners 828 may extend through respective standoffs 830 and a capture plate 832 which may fix the space between the two end plates 822A, B.

The example clutch assembly 86 may include a pressure plate 836. The pressure plate 836 may be robustly biased against a clutch plate stack 842 of the clutch assembly 86 by a bias member 838 (e.g. a wave spring). With the pressure plate 836 pressed against the clutch plate stack 842, the clutch assembly 86 may be in an engaged state and the rotor and stator components 844, 846 of the clutch plate stack 842 may be inhibited from rotating relative to one another. Any friction material may be used in the clutch plate stack 842, however, it may be preferable the material generate minimal dust and not be ferromagnetic.

As shown, the stem 816 of the drive screw 818 may extend through an aperture 840 in the pressure plate 836 of the clutch assembly 86. The drive screw 818 may include a flange 834 disposed at an end of the threaded region 826. The flange 834 may be shaped to be incapable of passing through the aperture 840 and may rest against the underside of the pressure plate 836. When the actuator 46 is actuated (rotated in the example embodiment), the drive screw 818 may rotate. As the end plate 822A is stationary, the drive screw 818 may translationally displace as it rotates due to its engagement with the threaded aperture 824 of the first end plate 822A. This, in turn, may drive the flange 834 into the pressure plate 836 and displace the pressure plate 836 in the direction of the actuator 46. The bias member 838 may become distorted as this occurs and pressure exerted on the clutch plate stack 842 may be relieved. Thus, the clutch assembly 86 may be disengaged and the rotor components 844 of the clutch plate stack 842 may be free to rotate relative to the stator components 846 of the clutch plate stack 842.

As shown, each of the rotor components 844 of the clutch plate stack 842 may include a toothed aperture 848. The toothed aperture 848 may accept and engage a cooperatively toothed segment 850 of a collet 852 such that the collet 852 rotates in unison with the rotor components 844 of the clutch plate stack 842. As best shown in FIG. 41B, an end of the leadscrew 84 disposed within the clutch assembly 86 may include a terminal projection 854 with a threaded region 856. When assembled, a collar 858 may be placed over the threaded terminal projection 854 and the collet 852 may be advanced into an interior opening of the collar 858. A nut 860 may be threaded onto the threaded region 856 until the collet 852 has been driven into the collar 858 such that the segmented end of the collet 852 is clamped against the exterior surface of the terminal projection 854. Thus, the leadscrew 84 and collet 852 may be inhibited from rotating relative to one another. Additionally, when the clutch assembly 86 is engaged, the leadscrew 84 may be prevented from rotating due to the interaction of the toothed segment 850 of the collet 852 and the toothed apertures 848 of each of the rotor components 844 of the clutch plate stack 842. A bearing 862 (e.g. a roller ball bearing) may be included to facilitate rotation of the leadscrew 84 with the clutch assembly 86 is in a disengaged state. The bearing 862 surrounds the terminal projection 854 and is retained between the second end plate 822B and the capture plate 832 in the example embodiment.

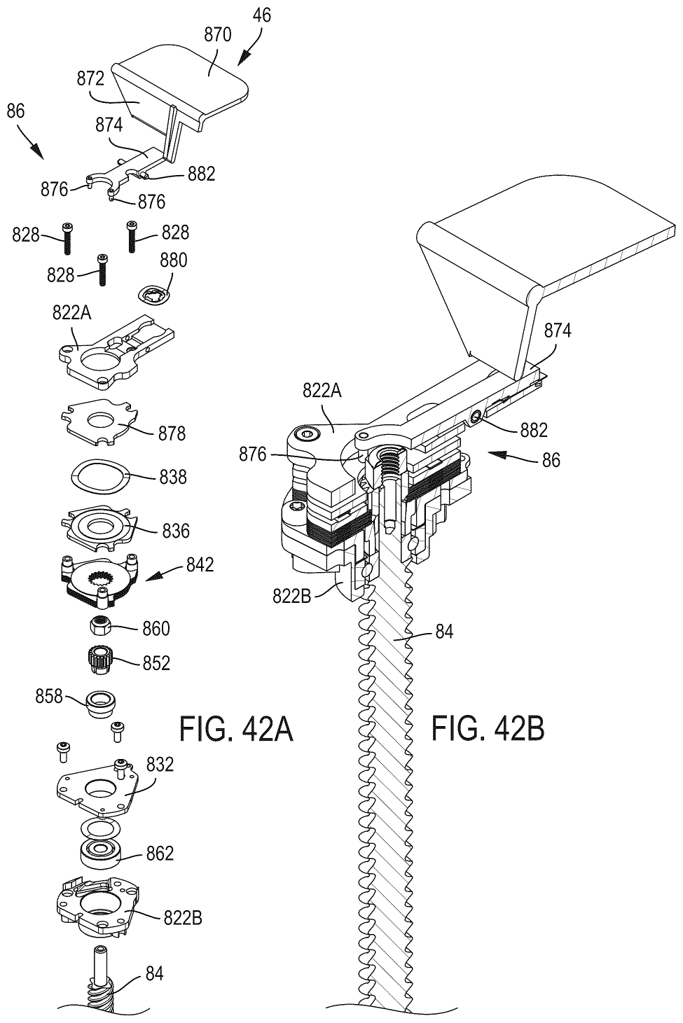

Referring now to FIGS. 42A-B, an exploded view and a cross-sectional view of another example clutch assembly 86 are respectively depicted. The clutch assembly 86 is similar to that shown in FIGS. 41A-B, but is actuated differently. As shown, an actuator 46 is depicted in FIGS. 42A-B. The example actuator 46 is a paddle type body which may be pivotally coupled within the drive head housing 250. The actuator 46 includes a press surface 870 and an actuator panel 872. A user may displace the press surface 870 to pivotally displace the actuator panel 872. As the actuator panel 872 is pivotally displaced, it may press against a tine 874 included in the clutch assembly 86.

As shown, the tine 874 may be pivotally coupled to a first end plate 822A of the clutch assembly 86 by a pivot pin 882. One end of the tine 874 may include a set of prongs 876 which may extend into contact with a toggle plate 878. The clutch assembly 86 may also include a pressure plate 836. A bias member 838 (e.g. wave spring) may be disposed intermediate the toggle plate 878 and the pressure plate 836. The toggle plate 878 may be displaceable within the clutch assembly 86 with respect to the pressure plate 836. To apply pressure to the clutch plate stack 842 and place the clutch assembly 86 in an engaged state, the toggle plate 878 may be driven toward the pressure plate 836 distorting the bias member 838. The distorted bias member 838 may press against the pressure plate 836 which in turn will apply pressure to the clutch plate stack 842. To disengage the clutch assembly 86, the bias member 838 may be allowed to displace the toggle plate 878 in a direction away from the pressure plate 838 to relieve pressure on the clutch plate stack 842.

In the exemplary embodiment, a tine bias member 880 is included. The tine bias member 880 may be disposed between the tine 874 and the first end plate 822A on a side of the pivot pin 876 opposite the prongs 876. The tine bias member 880 may urge the tine 874 to a resting state in which the prongs 876 press against the toggle plate 878 and drive the toggle plate 878 toward the pressure plate 836. Thus, the tine bias member 880 may ensure that the clutch assembly 86 is biased to an engaged state. As the actuator panel 872 is pivotally displaced against the tine 874, the actuator panel 872 may pivotally displace the tine 874 and compress the tine bias member 880. Pivotal displacement of the tine 874 may displace the prongs 876 away from the pressure plate 836. This may free the bias member 838 to restore to an uncompressed state as it urges the toggle plate 878 away from the pressure plate 836. Thus, interaction with the actuator 46 by a user may disengage the clutch assembly 86. The tine bias member 880 may automatically reengage the clutch assembly 86 when a user relieves pressure on the press surface 870 of the actuator 46.

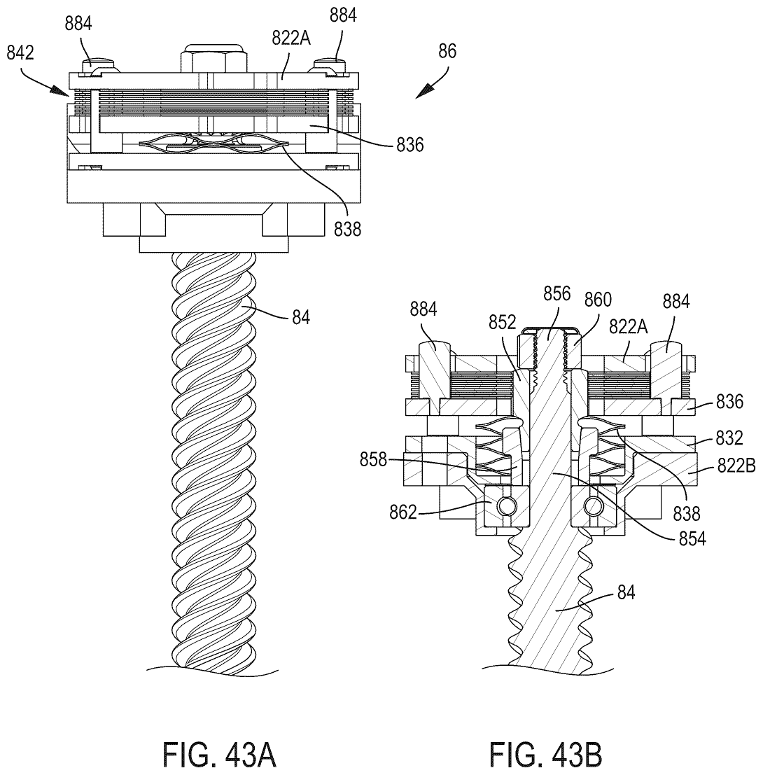

Referring now to FIGS. 43A-B, a side view and cross-sectional view of an alternative clutch assembly 86 are depicted. As with the embodiment shown in FIGS. 43A-43B, in certain examples, a toggle plate 878 may be omitted. In such examples, the pressure plate 836 may be disposed on a side of the clutch plate stack 842 opposite the first end plate 822A. The pressure plate 836 may be biased against the clutch plate stack 842 by a bias member 838 (e.g. a multi-wave compression spring) such that the clutch assembly 86 may default to an engaged state. The pressure plate 836 may include a set of drive pins 884 which extend through the clutch plate stack 842 and proud of the first end plate 822A. An actuator 46, either directly or through one or more intermediate parts, may be actuated to press against and displace the drive pins 884 and pressure plate 836 coupled thereto away from the first end plate 822A. This displacement may relieve pressure on the clutch plate stack 842 to disengage the clutch assembly 86. When the actuator 46 is released, the bias member 838 may urge the pressure plate 836 back against the clutch plate stack 842 to return the clutch assembly 86 to an engaged state.

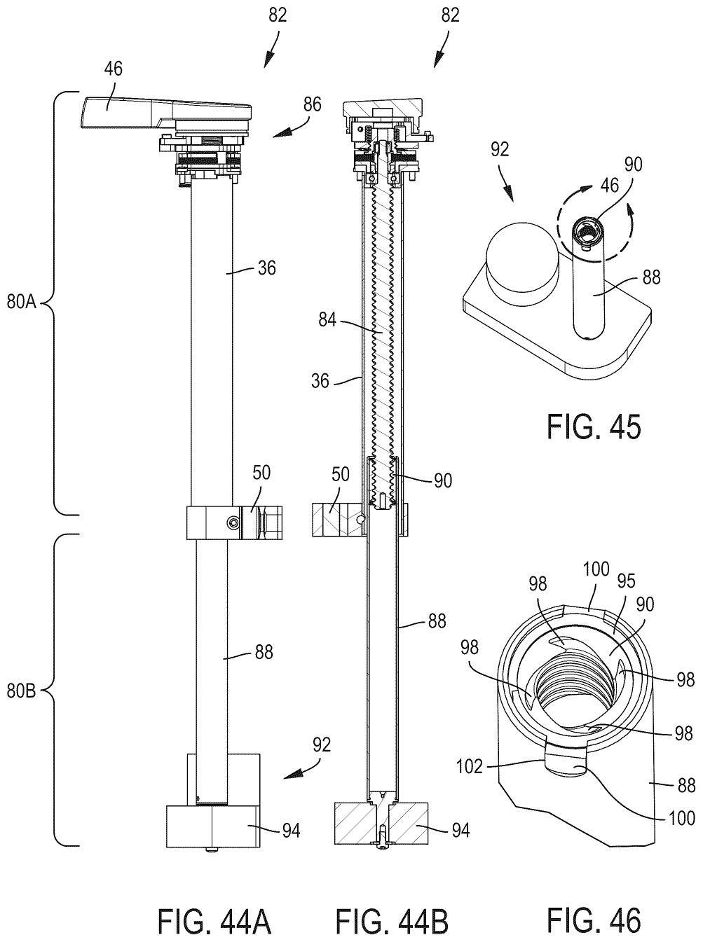

Referring now to FIGS. 44A-46, a number of views of an example drive assembly 82 are depicted. As shown, the drive assembly 82 may include a clutch assembly 86 (e.g. any of those described herein) which may be engaged and disengaged via displacement of an actuator 46. A leadscrew 84 is coupled to the clutch assembly 86 and may be locked from rotational displacement when the clutch assembly 86 is engaged. The leadscrew 84 may be permanently engaged with a nut 90 coupled to a nut tube 88 at an end thereof. The nut tube 88 may be coupled to a drive motor 92 via a transmission 94 (depicted schematically in FIGS. 44A-45). As the drive motor 92 is powered, the nut tube 88 may rotate. This may engender translational displacement of the leadscrew 84, drive head tube 36, and drive head 38 (see, e.g., FIG. 38A) when the clutch assembly 86 is in the engaged state. The end of the drive head tube 36 opposite the drive head 38 may have a carriage 50 coupled thereto. The carriage 50 may ride along and be guided by a bearing within the main housing 12 of the syringe pump 10 as the leadscrew 84 is displaced. As mentioned above with respect to FIGS. 38A-39B, with the clutch assembly 86 disengaged, the leadscrew 84 may be freely rotated. This may allow a user to manually adjust the position of the drive head 38 as desired thereby backdriving the leadscrew 84.

As best shown in FIG. 46, example nuts 90 may include a set of threads 98. In the example embodiment, the threads of the nut 90 are defined by four distinct thread starts and interface with a leadscrew 84 with threads defined by a like number of starts. The threads of the nut 90 and leadscrew 84 of any drive assembly 82 described herein may have threads defined by any desired number of starts (e.g. up to 8 or more). In various examples, the number of starts may be at least two (e.g. four or five). The number of starts may be determined in part by the efficiency of the leadscrew 84. In certain examples, e.g. where the nut 90 is a ball screw nut the screw may have a single thread start. The threads 98 of the nut 90 and cooperating threads of the leadscrew 84 may have a steep pitch of, for instance, 10 mm or greater (e.g. 12 mm-25 mm or more). This may facilitate manual displacement of the leadscrew 84, drive head tube 36, and drive head 38 when the clutch assembly 86 is in a disengaged state. In various examples, the nut 90 may be formed of a polymeric material. The leadscrew 84 may be formed of a metallic material such as stainless steel.

The nut 90 may be fixedly retained within the nut tube 88 in any suitable manner. The nut 90 may be coupled to the nut tube 88 via adhesive, retainer clips 95 (see, e.g., FIG. 46), interaction with retaining features included or formed in the nut tube 88, an overmolding operation, a swaging operation, heat stake, welding (e.g. sonic welding), etc. Though particular drive assembly 82 embodiments herein may be depicted with the nut 90 and nut tube 88 coupled in a particular manner, this is merely exemplary. In alternative embodiments of these examples, the nut 90 and nut tube 88 may be coupled together via any of the manners shown and described herein.

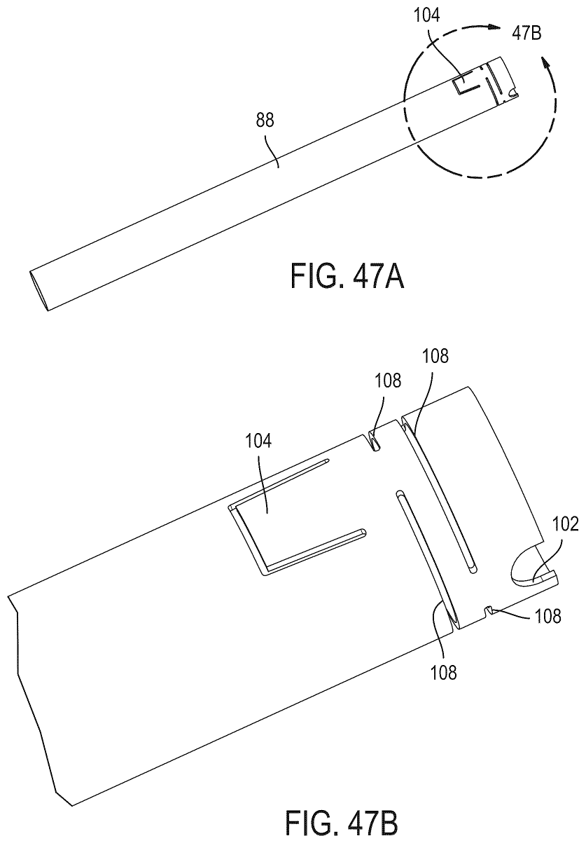

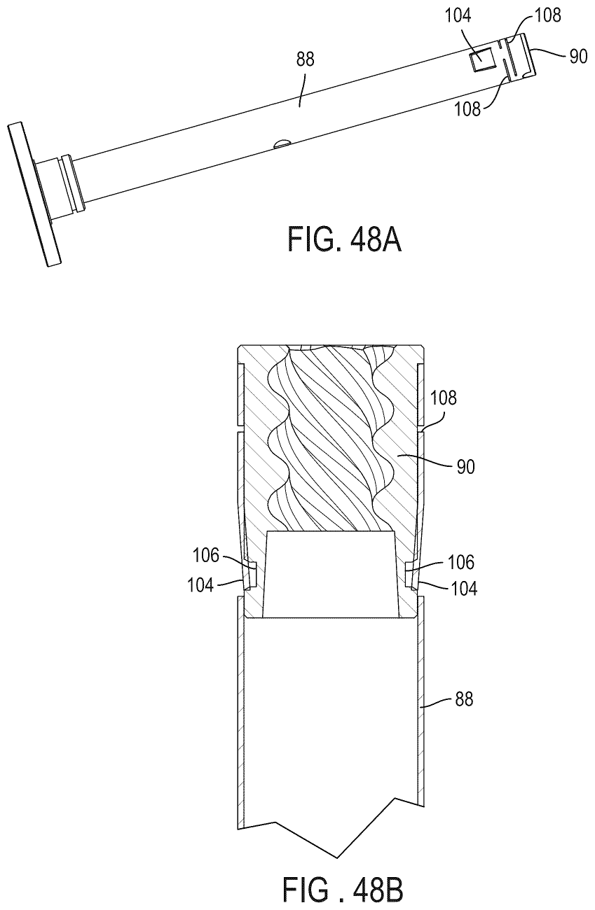

Referring now primarily to FIGS. 47A-48B, in some example drive assemblies 82, the nut tube 88 may include a set of cutouts at the end region of the nut tube 88 into which the nut 90 is installed. The cutouts may be formed in a laser cutting operation in various embodiments. The cutouts may define at least one cantilevered member or tine 104 in the wall of the nut tube 88. In preferred embodiments, cantilevered members 104 on opposing sides of the nut tube 88 may be formed by the cutouts. The cantilevered member(s) 104 may be bent inward toward the center axis of the nut tube 88 so as to form retaining barbs. The cutouts may also define a number of slots 108 intermediate the terminal end of the nut tube 88 and the cantilevered member(s) 104. In the example depicted in FIGS. 47A-48B, the nut tube 88 includes a first set of slots 108 disposed in opposition to one another proximal the terminal end of the nut tube 88. The nut tube 88 includes a second set of opposing slots 108 more distal to the terminal end of the nut tube 88. The first and second sets of opposing slots 108 may be angularly staggered from one another (e.g. 90° offset from one another). Additional sets of slots 108 may be included in alternative embodiments.

As best shown in the cross-sectional view depicted in FIG. 48B, the nut 90 may include a notch 106 or notches defined in its exterior side wall. As the nut 90 is displaced into the end of the nut tube 88, the cantilevered member(s) 104 may deflect. Once the nut 90 has been displaced beyond a certain distance into the nut tube 88, the cantilevered member(s) 104 may snap into the notch(es) 106. The presence of the cantilevered member(s) 104 in the notch(es) 106 may inhibit axial displacement of the nut 90 relative to the nut tube 88. The sets of slots 108 may act as an axial spring which is compressed as the nut 90 is driven into the end of the nut tube 88. This may add a preload which ensures the cantilevered member(s) 104 are firmly engaged with the notch(es) 106 inhibiting axial play between the nut 90 and nut tube 88.

The nut 90 may, in certain examples, include at least one projecting lobe 100 which projects radially from the exterior surface of the nut 90. The nut tube 88 may include a number of recesses 102 equal to the number of projecting lobes 100 on the nut 90. The projecting lobes 100 may be displaced into the recesses 102 during assembly to inhibit rotational displacement of the nut 90 relative to the nut tube 88.

In certain examples, the nut 90 may be slightly proud of the terminal end of the nut tube 88 when installed. The portion of the nut 90 proud of the nut tube 88 may be a flange region 110 of the nut 90. The flange region 110 may have a footprint equivalent to or larger than the nut tube 88. This may ensure the plastic forming the nut 90 contacts the drive head tube 36 before the nut tube 88 in the event that the nut tube 88 and drive head tube 36 are ever misaligned.

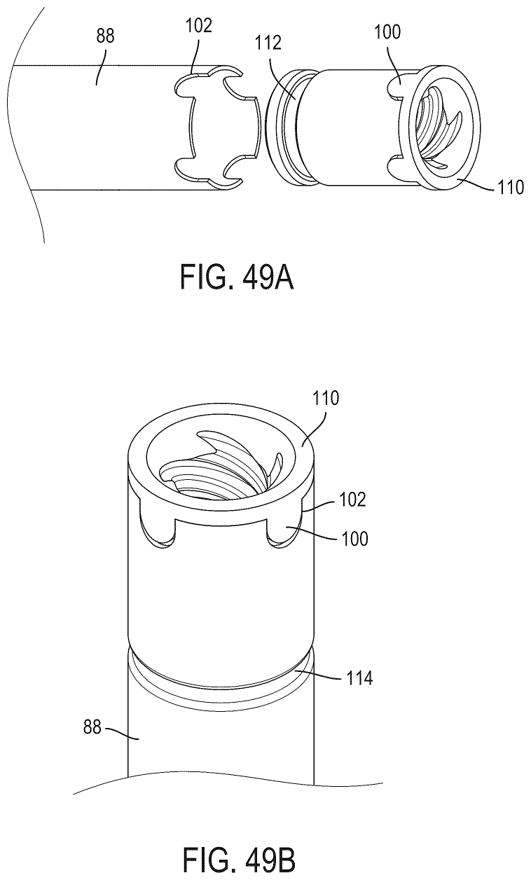

Referring now to FIGS. 49A-49B, in examples where the nut 90 is retained via a swaging operation, a recess 112 may extend around the exterior surface of the nut 90. Once the nut 90 is positioned in the nut tube 88, the nut tube 88 material may be deformed into the recess 112. This may be accomplished by rotating the nut tube 88 while driving rollers of a knurler or swaging assembly into the region of the nut tube 88 surrounding the recess 112 in the exterior surface of the nut 90. This may generate a depressed band 114 in the nut tube 88 surface that extends into the recess 112 axially locking the nut 90 in place with respect to the nut tube 88. The nut 90 may be rotationally locked by locating projecting lobes 100 of the nut 90 within recesses 102 in the end of the nut tube 88.

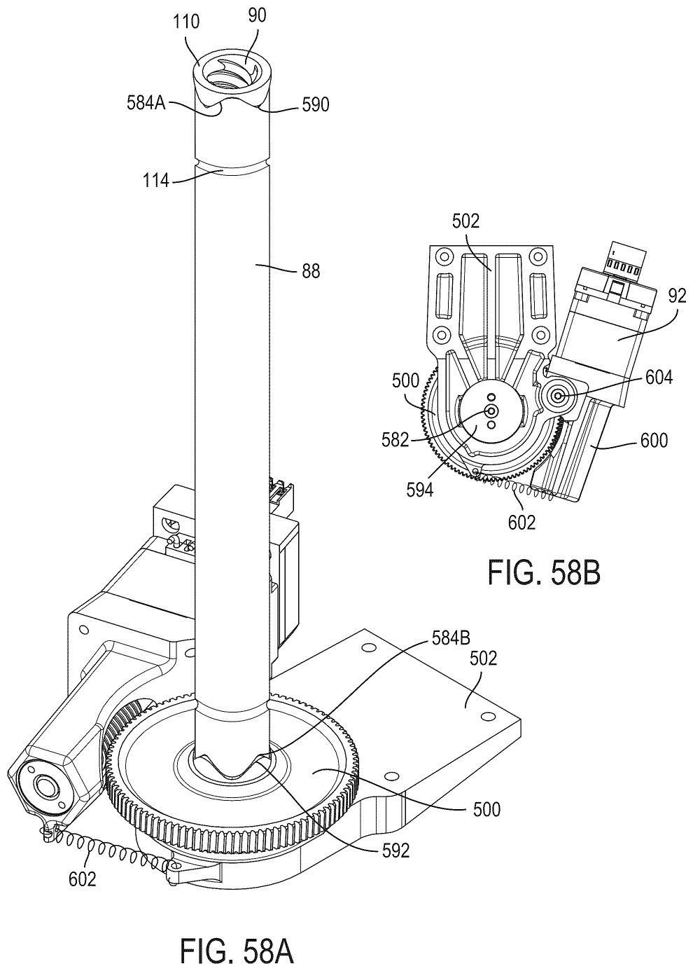

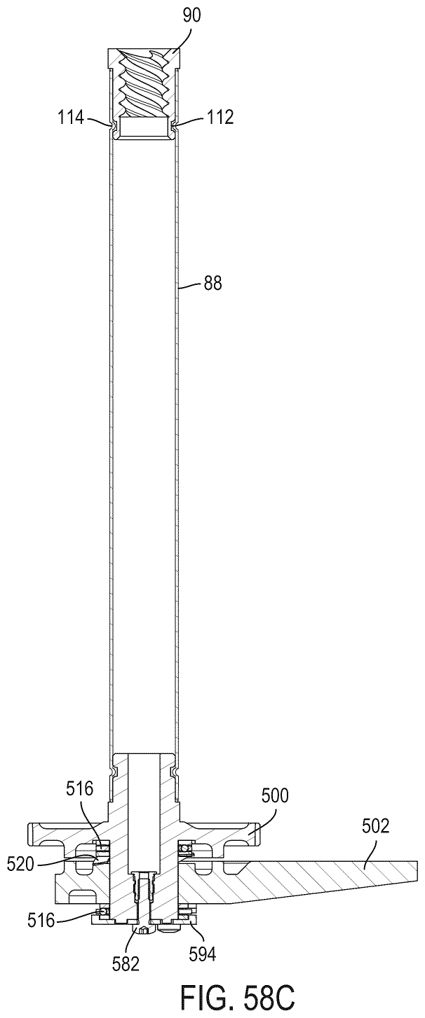

Referring now to FIGS. 50-58C, a variety of driving portions 80A for drive assemblies 82 are depicted. In various exemplary embodiments, the drive assembly 82 may be arranged so as to be non-backdrivable. In certain examples, the transmission 96 which transfers motion from the drive motor 92 (see, e.g., FIG. 58C) to the nut tube 88 may be a worm drive, though other non-backdrivable arrangements may also be utilized in the alternative. In the examples shown in FIGS. 50-58C each of the nut tubes 88 is coupled to a worm wheel 500 merely as an example (drive motors 92 and transmissions 96 are not depicted in all embodiments). It should be understood that other varieties of gears may be substituted for the worm wheel 500 in embodiments which do not include a worm drive. The example drive assembly 82 sections may couple to a mounting plate 502. In turn, the mounting plate 502 may be anchored to a housing 12 of the syringe pump 10 in any suitable manner (e.g. set of fasteners). Bearings (e.g. thrust washers, rolling element bearings, roller thrust bearings, roller ball thrust bearings) between the worm wheel 500 and mounting plate 502 may be included.

Still referring to FIGS. 50-58C, driving portions 80B for certain example drive assemblies 82 may include one or more relative displacement lock. For example, certain example may include at least one relative rotation lock. A relative rotation lock may substantially prevent relative rotation between the worm wheel 500 and the nut tube 88 in at least one direction. Additionally, driving portions 80B may further include at least one relative translation lock. A relative translation lock may substantially prevent relative displacement of the worm wheel 500 and nut tube 88 along the axis of the nut tube 88 in at least one direction. In certain examples, a relative rotation lock may also act as a relative translation lock. Additionally, the nut tube 88 and worm wheel 500 may be substantially blocked from translational displacement with respect to the mounting plate 502 by at least one locator. In some embodiments, a locator may also act as a relative translation lock inhibiting relative translational displacement between the nut tube 88 and worm wheel 500 in at least one direction. Though certain locators or relative displacement locks are shown in relation to particular embodiments herein, it should be understood that these embodiments are mere examples. Alternative embodiments may substitute shown locators and relative displacement locks for others described herein.

In various embodiments, at least one bias member 520 may be included. The bias member 520 may exert a force against the coupled nut tube 88 and worm wheel 500 which biases these components in the direction of the drive head 38. Thus, the drive assembly 82 may have high stiffness and resist any movement due to force exerted by a syringe plunger 24 against the drive head 38 as fluid is urged out of the syringe 18. Additionally, the bias member 520 may allow some compliance in the drive assembly 82 in the event that the syringe pump 10 is improperly handled. For example, if the syringe pump 10 is dropped such that the drive head 38 first makes contact with the ground, the bias member 520 may assist in mitigating effects to the drive assembly 82.

Referring now primarily to FIG. 50, an exploded view of an example section of a driving portion 80B of a drive assembly 82 is depicted. As shown, a driving portion 80B may include a nut tube 88 to which a nut 90 (see, e.g., FIG. 49A) may be coupled. The nut tube 88 may include a number of apertures 504 disposed in an end region of the nut tube 88. The apertures 504 may be spaced at regular angular intervals, but need not be in all embodiments. Three are shown in the example. The driving portion 80B may further include a worm wheel 500. The worm wheel 500 may include a teethed disk portion 506 and central bore 508 which extends through the worm wheel 500. A wall 510 may surround the periphery of the central bore 508. The wall 510 may include a number of passages 512 extending therethrough that may be countersunk. The passages 512 may be provided at intervals which mimic those of the apertures 504 in the nut tube 88. The nut tube 88 may couple to the worm wheel 500 via a number of rivets 514 which may be installed through the paired sets of apertures 504 and passage 512. The rivets 514 may provide a relative rotation lock and relative translation lock which constrain the nut tube 88 and worm wheel 500 such that the two move in tandem with one another.

Still referring primarily to FIG. 50, the driving portion 80B may also include a set of bearings 516. Any variety of bearing 516 may be included, however, thrust washers are depicted. One bearing 516 may be included on each side of the mounting plate 502. A bias member 520 (e.g., wave spring) may be disposed between the mounting plate 502 and the bearing 516 on the side of the mounting plate 502 most proximate the teethed disk portion 506 of the worm wheel 500. As mentioned above, the bias member 520 may increase the stiffness of the drive assembly 82 and help mitigate issues related to improper handling. Other embodiments of driving portions 80B shown herein may include bias members 520 and bearings 516 which may be similarly assembled into those respective embodiments.

When assembled, a portion of the nut tube 88 may extend through an aperture 522 in the mounting plate 502. A retainer body 518 may be coupled in place on a portion of the nut tube 88 extending through the mounting plate 502. This may inhibit relative displacement of the nut tube 88 and worm wheel 500 with respect to the mounting plate 500. Thus, the retainer body 518 may act as a locator. The retainer body 518 may be substituted by any of the other locators described herein.

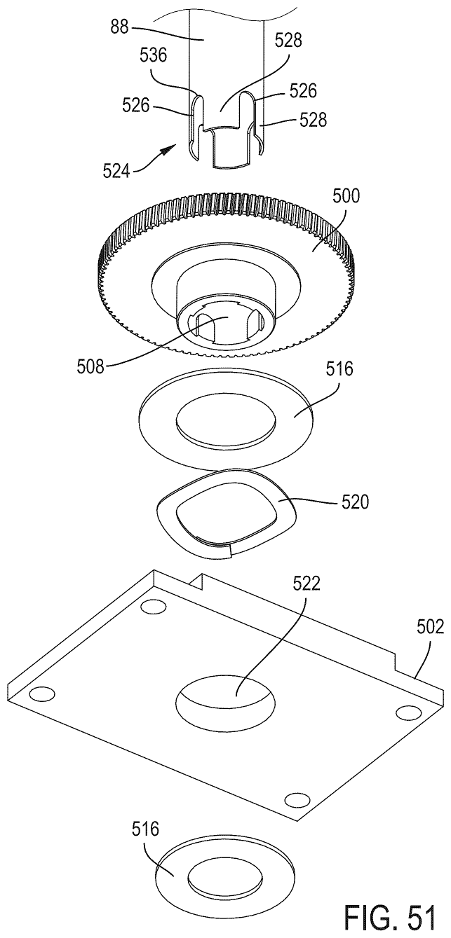

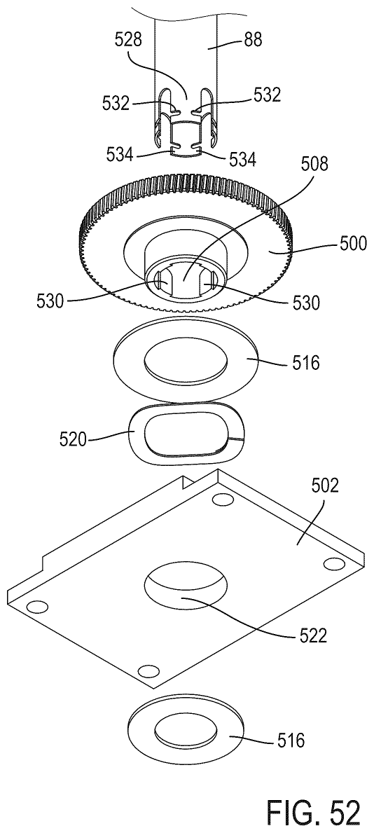

Referring now to FIGS. 51-52, exploded views of two additional example sections of driving portions 80B are depicted. As shown, the terminal region 524 of a nut tube 88 may include a number of notches 526. The notches 526 may be spaced at regular angular intervals though need not be in all embodiments. Between the notches 526 may be a number of nut tube panels 528. The central bore 508 through the worm wheel 500 may include a number of projecting regions 530 present on the wall of the bore 508 (best shown in FIG. 52). The projecting regions 530 may be spaced at like intervals to the notches 526 in the nut tube 88 and may be sized to snuggly fit into the notches 526 when the drive assembly 82 is assembled. Engagement between the projecting regions 530 and the notches 526 may provide a relative rotation lock which inhibits rotational displacement of the worm wheel 500 relative to the nut tube 88. Additionally, the projecting regions 530 may bottom out against the end 536 of the notches 526, thus, the engagement of the projecting regions 530 with the notches 536 may block relative translation in one direction.

The length of the notches 526 may be selected such that a portion of the panels 528 extend beyond the central bore 508 and through the mounting plate 502. When assembled, the exposed portion of each panel 528 may be distorted so as to provide a relative translation lock that inhibits translational displacement of the worm wheel 500 with respect to the nut tube 88. With respect to FIG. 51, the exposed portion of each panel 528 may be swaged over the bearing 516 on side of the mounting plate 502 opposite the worm wheel 500. With respect to FIG. 52, the panels 528 include slits 532 which leave at least one portion of each panel 528 cantilevered to the remainder of the panel 528. When assembled, the cantilevered tabs 534 may be bent over the bearing 516 on the side of the mounting plate 502 opposite the worm wheel 500. The distortion of the panel in FIG. 51 and FIG. 52 may also act as a locator which prevents the coupled nut tube 88 and worm wheel 500 from displacing relative to the mounting plate 502. In some embodiments, the slits 532 may be made such that the tabs 534 have a ramped face. When the tabs 534 are bent over the bearing 516, the ramped face may assist in limiting any translational play between components.

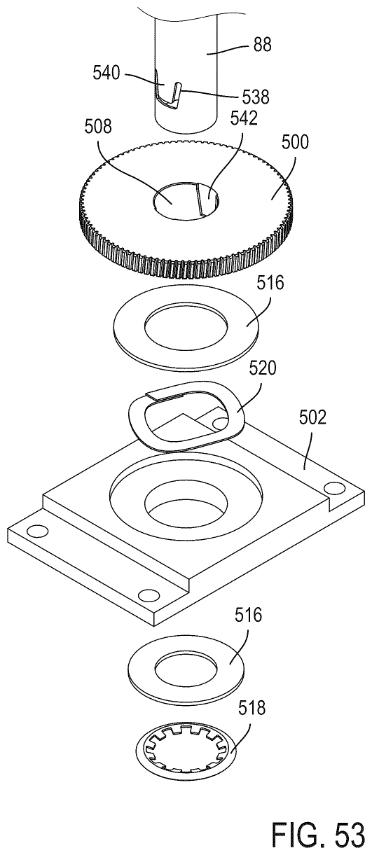

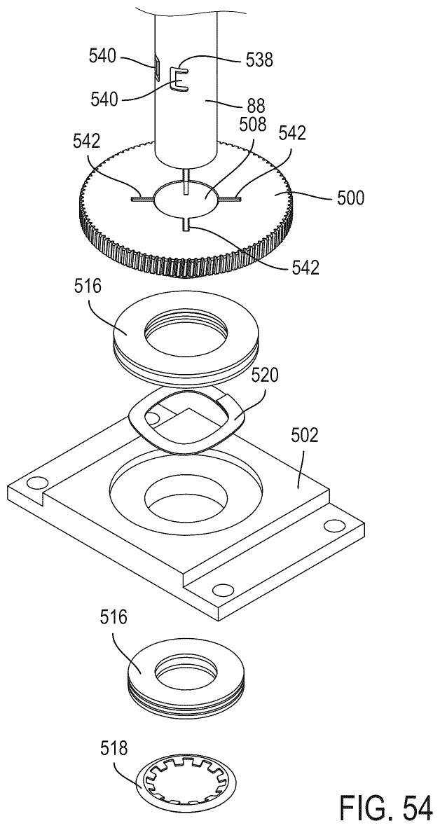

Referring now to FIG. 53 and FIG. 54, exploded views of two additional example sections of driving portions 80B are depicted. As shown, each nut tube 88 includes a set of cutouts 538 which define tabs 540 in the nut tube 88. These tabs 540 may be bent outward away from the axial dimension of the nut tube 88. The worm wheel 500 may include a number of receptacles 542 which may accept the tabs 540. The engagement of the tabs 540 with the receptacles 542 may prevent certain relative displacement between the nut tube 88 and the worm wheel 500.

As shown in FIG. 53, the central bore 508 through the worm wheel 500 includes two recessed regions in the wall of the central bore 508 which form the receptacles 542. With the tabs 540 bent out away from the axis of the nut tube 88, the worm wheel 500 may be slid over the nut tube 88 and into the tabs 540 at the point where the tabs 540 are connected to the remainder of the nut tube 88. Further displacement of the worm wheel 500 may elastically deflect the tabs 540 such that the worm wheel 500 may continue to displace along the nut tube 88. The tabs 540 may elastically restore into the recessed regions when the worm wheel 500 is advanced beyond a certain point and appropriately aligned. The recessed regions may be arranged to prevent the tabs 540 from fully restoring to a relaxed state such that they firmly press against the recessed regions when the drive assembly 82 is assembled. Engagement of the tabs 540 with the recessed regions may provide a relative rotation lock which inhibits displacement of the worm wheel 500 relative to the nut tube 88. This engagement may also prevent the worm wheel 500 from translationally displacing toward the nut 90 (see, e.g., FIG. 49A).

Referring primarily to FIG. 54, a number of channels may extend outwardly with respect to the central bore 508 of the worm wheel 500 to form the receptacles 542. The channels may not extend through the entirety of the worm wheel 500. In the example embodiment, the channels extend substantially in a radial direction with respect to the central bore 508 though need not do so in alternative embodiments. With the tabs 540 bent outward, the nut tube 88 may be slid into the central bore 508 of the worm wheel 500 until the tabs 540 bottom out in the channels. With the tabs 540 bottomed out, further relative translation in this direction may be prevented. Additionally, the engagement of the tabs 540 in the channels may provide a relative rotation lock which blocks relative rotation between the worm wheel 500 and nut tube 88. As shown, the tabs 540 in the nut tube 88 of FIG. 54 are arranged such that they must be swung in opposing directions when being bent to project outwardly from the rest of the nut tube 88. This may assist in limiting any play in the relative translation lock provided by the interaction of the tabs 540 and channels.

As with other example embodiments described herein, a locator 518 may be included to prevent relative translation between the coupled nut tube 88 and worm wheel 500 and the mounting plate 502. As shown in FIGS. 53-54 a fingered retention ring may be utilized, however, any other locator 518 described herein may be substituted in alternative embodiments.

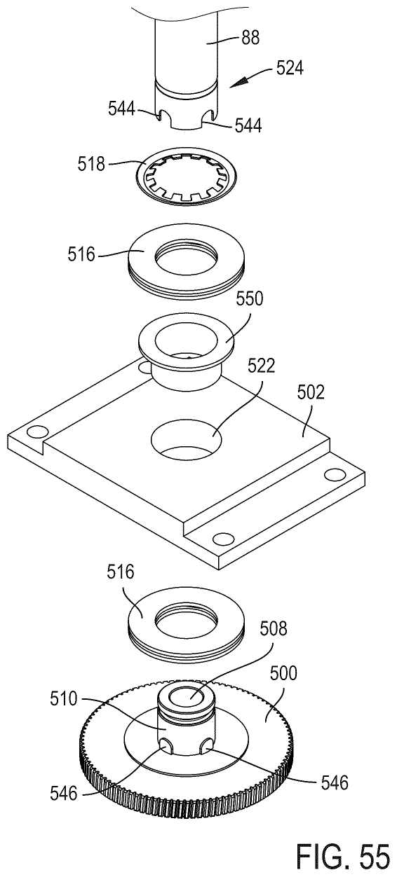

Referring now to FIG. 55, in certain embodiments, the worm wheel 500 may be positioned on a side of the mounting plate 502 opposite the nut 90 (see, e.g., FIG. 58C) attached to the nut tube 88. As shown, a terminal region 524 of the nut tube 88 opposite the nut 90 may include a number of notches 544. The exterior face of the wall 510 surrounding the periphery of the central bore 508 in the worm wheel 500 may include a number of projecting lobes 546. When the drive assembly 82 is assembled, the nut tube 88 may be fed through the aperture 522 in the mount plate 502 and the wall 510 may be seated into the end of the nut tube 88. The lobes 546 may be aligned with and received within the notches 544 at the terminal region of the nut tube 88. This may provide a relative rotation lock between the worm wheel 500 and the nut tube 88. Additionally, as the lobes 546 may be bottomed out at the ends of the notches 544, a relative translation lock may be provided as a mechanical interference may be presented that blocks further relative translation. As shown, the central wall 510 also includes a recess 548 which extends around the exterior surface of the central wall 510. The nut tube 88 may be distorted (e.g. knurled or swaged) into this recess 548 to provide an additional relative translation lock substantially blocking relative translation between the nut tube 88 and the worm wheel 500 along the axial dimension of the nut tube 88. A locator 518 (in the example a retaining ring) is included to inhibit relative translation between the coupled nut tube 88 and worm wheel 500 and the mounting plate 502. In various example driving portion 80B embodiments, a sleeve bearing 550 may be provided in the aperture 522 of the mounting plate 502.

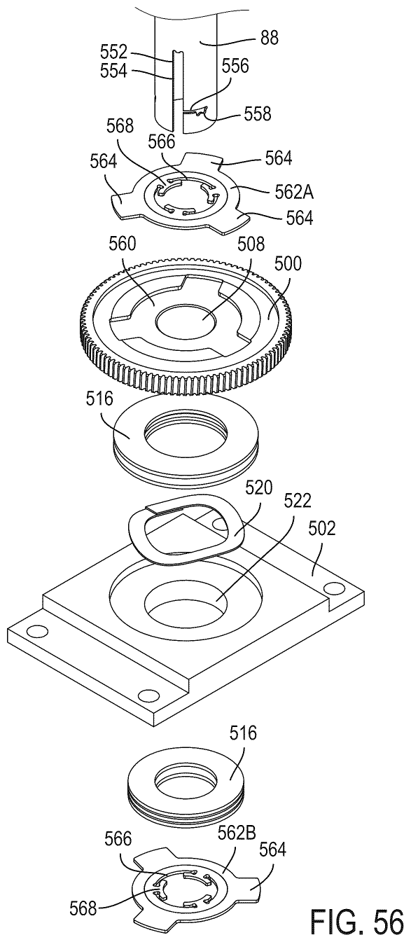

Referring now to FIG. 56, an exploded view of yet another example section of a driving portion 80B is depicted. As shown, a nut tube 88 may include a set of slots 552 cut into a terminal end of the nut tube 88. Each slot may include a main portion 554 and a branch 556. The branch 556 may extend at an angle substantially perpendicular to the main portion 554. An end region of each branch 556 most distal to the main portion 554 may include a detent 558. The worm wheel 500 may include a keyed receptacle 560 which surrounds the central bore 508. As shown the driving portion may also include a set of keyed coupling bodies 562A, B. In the exemplary embodiment, the coupling bodies 562A, B are identical, though need not be in all embodiments.

As shown, each coupling body 562A, B may be substantially planar and may include a set of spokes 564 projecting from the periphery of the coupling body 562A, B. The spokes 564 may be spaced so as to cooperate with the keying feature of the keyed receptacle 560 in the worm wheel 500. A passage 566 may extend through the center of each coupling body 562A, B. A number of prongs 568 may extend from the wall of each passage 566 toward the center of the passage 566. The prongs 568 shown are substantially in the shape of the Latin character “T” in the example embodiment, though may differ in alternative embodiments.

When assembled, coupling body 562A may be aligned with keyed receptacle 560 and seated into the keyed receptacle 560. The nut tube 88 may then be fed through the central bore 508 in the worm wheel 500. The nut tube 88 may be oriented such that the prongs 568 of the coupling body 562A enter into and travel along the main portions 554 of respective slots 552 in the end of the nut tube 88. The nut tube 88 may be advanced until the prongs 568 contact the ends of the main portions 554 of the slots 552. The prongs 568 may prevent the coupling body 562A from rotating relative to the nut tube 88. Likewise, the keyed nature of the engagement between coupling body 562A and the keyed receptacle 560 may block relative rotation between the worm wheel 500 and the nut tube 88. Thus a relative rotation lock may be provided. As the prongs 568 may be pressed against the ends of the main portions 554 of the slots 552 relative translation in one direction may also be blocked.

A portion of the nut tube 88 may be fed through the aperture 522 in the mounting plate 502. To block relative translation in the opposing direction, the prongs 568 of the second coupling body 562B may be displaced into respective slots 552. When the bias member 520 is in a resting state, access to the branches 556 may be blocked by another part of the driving portion 80B. The bias member 520 may be stressed (e.g. in a fixture) to allow components of the driving portion 80B to displace a distance sufficient to make the branches 556 accessible and the coupling body 562B may be rotated such that the prongs 568 pass into respective branches 556. As the prongs 568 reach the detents 558, the bias member 520 may be allowed to restore to a less stressed state. This may provide a relative translation lock which inhibits translational displacement between the nut tube 88 and the worm wheel 500. Additionally, this may provide a locator which inhibits relative displacement of the nut tube 88 and worm wheel 500 with respect to the mounting plate 502. Such an arrangement may be desirable as it may help facilitate field replacement of the drive assembly 82 components.

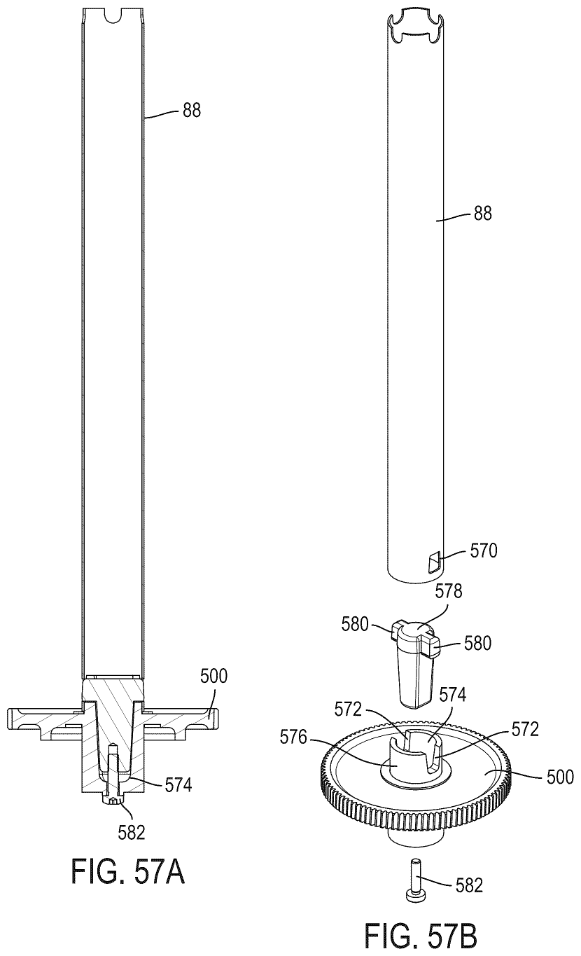

Referring now to FIG. 57A and FIG. 57B, a cross-sectional and exploded view of another section of an example driving portion 80B are respectively depicted. As shown, a nut tube 88 may include a set of opposing fenestrations 570. The worm wheel 500 may include a central pocket 574 instead of a central bore 508 (see, e.g., FIG. 53). A wall 576 including a set of opposing troughs 572 may be provided around the periphery of the pocket 574. A peg 578 including a set of opposing cars 580 may also be included. The peg 578 may be seated into the pocket 574 of the worm wheel 500 and the cars 580 of the peg 578 may be displaced into the troughs 572 of the wall 576. Though the interaction between the cars 580 and the troughs may prevent relative rotation between the peg 578 and worm wheel 500, in some embodiments the peg 578 and pocket 574 may also be cooperatively keyed. As best shown in FIG. 57A, a fastener 582 may extend into the pocket 574 and into engagement with the peg 578 to couple the peg 578 and worm wheel 500. The fastener 582 may inhibit relative translation between the peg 578 and worm wheel 500. The end of the nut tube 88 adjacent the fenestrations 570 may be pressed against the cars 580. The cars 580 may include a ramped face which facilitates displacement of the end of the nut tube 88 over the cars 580. The cars 580 may snap into engagement with the fenestrations 570 when the nut tube 88 is appropriately aligned with the cars 580 and advanced an appropriate distance. This may both provide a relative rotation and translation lock between the worm wheel 500 and the nut tube 88. Though not shown, various bearings 516 and a bias member 520 may be included to facilitate attachment to a mounting plate 502 as described in relation to other example embodiments herein.

Referring now to FIG. 58A, FIG. 58B, and FIG. 58C, a cross-sectional view and a perspective view, a plan view, and a cross-sectional view of an example driving portion 80B of a drive assembly 82 are respectively depicted. As shown, the nut 90 may be inserted into the nut tube 88 and a band 114 of nut tube 88 material may be deformed into a recess 112 defined in the exterior surface of the nut 90. This may be done as described in relation to FIGS. 49A-B. With the band 114 deformed, axial displacement of the nut 90 relative to the nut tube 88 may be inhibited. The worm wheel 500 may include a stem 586 (best shown in FIG. 58C). The stem 586 may include a recess 588 in an exterior face of the stem 586. A band 114 of nut tube material may also be deformed into the recess 588 in the stem 586. This may provide a relative translation lock between the nut tube 88 and the worm wheel 500.

The ends of the nut tube 88 may each include an undulating surface 584A, B. The undulating surface 584A on one end of the nut tube 88 may cooperate with a complimentary undulating surface 590 defined on a flange region 110 of the nut 90. When the complimentary undulating surfaces 584A, 590 are seated against one another, relative rotation of the nut 90 and nut tube 88 may be inhibited. The undulating surface 584B on the opposing end of the nut tube 88 may cooperate with a complimentary undulating surface 592 on a portion of the worm wheel 500. The engagement of undulating surfaces 584B, 592 may provide a relative rotation lock between the worm wheel 500 and the nut tube 88. A portion of the worm wheel 500 may extend through an aperture 522 in the mounting plate 502. A cap 594 may be included and may be coupled to the worm wheel 500 via a fastener 582. A bearing 516 may be sandwiched between the cap 594 and the mounting plate 502. The fastener 582, cap 594, and bearing 516 may hold the coupled nut tube 88 and worm wheel 500 from displacing translationally with respect to the mounting plate 502 and act as a locator.

Referring now primarily to FIG. 58A and FIG. 58B, the worm wheel 500 may interdigitate with a worm gear 598 powered by a drive motor 92. The drive motor 92 and worm gear 598 may be coupled to the mounting plate 502 via a pivotal coupling 604 in certain examples. The worm gear 598 may be at least partially surrounded by a housing 600 coupled to the drive motor 92. A bias member 602 (e.g. extension spring) may be included and may exert a force against the housing 600 which tends to pivot the worm gear 598 toward the worm wheel 500. Thus, the drive assembly 82 may automatically compensate for any wear that occurs over the life of the drive assembly 82. It may also assist in creating a very low backlash gearing arrangement. The worm wheel 500 and worm gear 598 may also be self-locking or non-backdrivable.

Referring now to FIGS. 59A-60D, in various alternative embodiments, a drive assembly 82 may include a nut assembly 140 which may be actuatable between engaged and disengaged states with respect to a motor 92 driven leadscrew 84 (leadscrew threads not shown). In some embodiments, the nut assembly 140 may be a collet nut type assembly as described below. Such a nut assembly 140 may be relatively compact and be disposed coaxial with the leadscrew 84. Thus, as with other drive assembly 82 embodiments described herein, a drive assembly 82 with such a nut assembly 140 may be particularly stiff. Larger and stiffer nut assemblies 140 and leadscrews 84 may be utilized, increasing stiffness of the drive assembly 82. Additionally, the coaxial disposition of the nut assembly 140 may limit offset loading as the drive assembly 82 is operated. Such nut assemblies 140 may also facilitate the use of an in-line motor which directly drives the leadscrew 84.

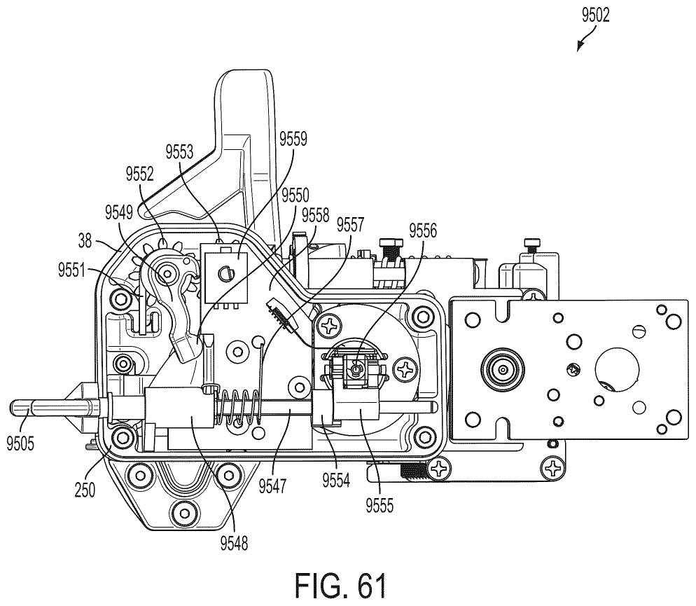

Example nut assemblies 140 may include a stationary portion. At least one arm 146, 186 may be connected to the stationary portion and may be displaceable (e.g. pivotable or deflectable) relative to the stationary portion. The arm(s) 146, 186 may include a threaded region with thread features which cooperate with the leadscrew 84. The arm(s) 146, 186 may also include at least one interface surface defined on a portion of each arm 146, 186. Example nut assemblies 140 may also include an actuation subassembly 158, 192. The actuation subassembly 158, 192 may be coupled to an actuator 178. The actuator 178 may be displaced to translationally drive the actuation subassembly 158, 192 between a first and second position relative to the stationary portion of the nut assembly 140 (further described in relation to FIGS. 61-63B). At least one bias member may be included and may urge the actuation subassembly 158, 192 to one of the first and second positions depending on the embodiment. The actuation subassembly 158, 192 may include at least one guide surface. As the actuation subassembly 158, 192 is displaced from the first position toward the second position, the at least one guide surface may displace the arm(s) 146, 186, via interaction with the interface surface, toward the center axis of the nut assembly 140 (and any leadscrew 84 extending therethrough). This may lead the threaded region of the arm(s) 146, 186 to be pressed into engagement with the threads of a leadscrew 84. As the actuation subassembly 158, 192 is displaced toward the first position, the at least one guide surface may displace the arm(s) 146, 186, via contact with the interface surface, away from the center axis of the nut assembly 140 (and out of engagement with any leadscrew 84 extending therethrough). Alternatively or additionally, the arm(s) 146, 186 may be associated with a return spring that may urge the arm(s) 146, 186 away from the center axis of the nut assembly 140 when the actuation assembly 158, 192 is displaced toward the first position.

In the example embodiment depicted in FIGS. 59A-E, the nut assembly 140 includes a nut body 142 and an actuation subassembly 158. The actuation subassembly 158 is coupled to an actuator 178 which may be actuated to cause displacement of the actuation subassembly 158 with respect to the nut body 142. The nut assembly 140 may further include at least one bias member 164. The bias member(s) 164 may urge the actuation subassembly 158 in a first direction to a position in which the actuation subassembly 158 presses threads on the nut body 142 in a direction toward the center axis of the nut assembly 140. In use, this may be a direction in which the threads are pushed into engagement with threads of a leadscrew 84 of the drive assembly 82. The actuator 178 may be actuated to displace the actuation subassembly 158 in a second direction opposite the first direction to distort the bias members 164. As the actuation subassembly 158 is displaced in the second direction, threaded region(s) of the nut body 142 may be driven away (e.g. deflected) from the center axis of the nut assembly 140. In use, this movement would displace the threaded regions away from the leadscrew 84 to a disengaged state. The bias members 164 may automatically displace the actuation subassembly 158 back in the first direction when the force on the actuator 178 is relieved. Thus example nut assemblies 140 may be biased to an engaged state with the leadscrew 84.

In alternative embodiments, the opposite arrangement may be employed. The bias members 164 may urge the threaded regions of the nut body 142 to a disengaged state. The actuator 178 may be manipulated to drive the actuation subassembly 158 in a direction which forces the threaded regions into engagement with leadscrew 84.

In certain examples, a nut body 142 may be constructed of a polymeric material and may, for example, be injection molded. The nut body 142 may include a base 144 and at least one arm 146 having a threaded region spaced from the base 144. Though two arms 146 are depicted in the example embodiment, alternative embodiments may include a greater or lesser number. The base 144 may be a stationary portion of the nut assembly 140 and may include a bore 148 which extends through the central region of the base 144 along the axis of the nut body 142. The bore 148 may have a width greater than that of the leadscrew 84. Thus, the walls of the bore 148 may be out of contact with the leadscrew 84 when a drive assembly 82 including the nut assembly 140 is in an assembled state. The base 144 may include flanges 160 at opposing ends of the base 144. Each arm 146 may include at least one projection 152 which extends into a respective track 170 defined on a body 162 disposed in flanking relation to the arm 146. The body 162 may form part of the actuation subassembly 158 and the track 170 may be extended along a path which forces the arm 146 to pivot toward and away from the axis of the nut assembly 140 as the actuation subassembly 158 displaces translationally with respect to the nut body 142. That is, the sidewalls of the track(s) 170 may from guide surfaces which interact with interface surfaces defined on the respective projection(s) 152 to alter the position of each arm 146 as the actuation subassembly 156 displaces relative to the base 144 of the nut body 142.

Two arms 146 which extend in a cantilevered manner from the base 144 and are disposed in opposition to one another are included in the example embodiment depicted in FIGS. 59A-E. Where a greater number of arms 146 are included, they may be disposed at regular angular intervals. Each of the arms 146 may include a terminal region 150. The faces of the terminal regions 150 of the arms 146 facing the interior of the nut body 142 or axis of the nut assembly 140 may be threaded so as to cooperate with a desired leadscrew 84 (e.g. any of those described elsewhere herein). As shown, in certain embodiments, at least one projection 152 may extend from the terminal regions 150 of the arms 146. In the example embodiment, each arm 146 includes two projections 152 each disposed on opposing substantially flat lateral surfaces 154 of the arms 146. The projections 152 may extend perpendicular to the respective lateral surfaces 154 and in a direction skew to the axis of the nut body 142, but substantially parallel to a line perpendicular to that axis.

In various example embodiments, each arm 146 may also include a slit 156 extending through the arm 146. The slit 156 in each arm 146 may extend from one lateral surface 154 of the arm 146 through the arm 146 to the opposing lateral surface 154 of that arm 146. The slits 156 may extend along the arms 146 in a direction substantially parallel to the axis of the nut body 142. Each slit 156 may span from a portion of each arm 146 adjacent the base 144 to a point on the respective arm 146 more proximate to or within the terminal region 150 of that arm 146. Each end of example slits 156 may be enlarged and rounded (see, e.g., FIG. 59C).

The example actuation subassembly 158 shown in FIGS. 59A-E includes a retainer body 161, a number of flanking bodies 162, and at least one bias member 164. In the example embodiment, a set of bias members 164 are included and are depicted as compression springs. The retainer body 161 may form an end of the actuation subassembly 158 and each flanking body 162 may mate into the retainer body 161. At least some of these components may be combined into a monolithic structure in various examples. Each exemplary flanking body 162 may include an interior face 166 which may seat against or adjacent a set of lateral faces 154 of arms 146 of the nut body 142. The interior faces 166 may include a trough or recess 168. The trough 168 in each flanking body 162 may be disposed along the central region of the interior face 166 and extend across the entirety of the interior face 166 in a direction parallel to the axis of the nut body 142. The trough 168 may provide clearance to ensure that the leadscrew 84 is out of contact with the flanking bodies 162 when the nut assembly 140 is in an assembled state.

Each of the flanking bodies 162 may include at least one track 170 as mentioned above. In the example embodiment, each flanking body 162 includes a track 170 for a projection 152 on each of the arms 146. The tracks 170 are disposed at a non-parallel angle to one another. Though straight tracks 170 are depicted, tracks 170 which are curved or include a curved region are possible. Additionally, tracks 170 which start at a first angle and redirect at least once to a different angle may also be possible. It may be preferred that actuation of the arms 146 (or at least the majority of the actuation) occur over a steeper angled section of each track 170. A shallower angled section may be included to allow extra travel to accommodate any wear without substantially effecting performance of the nut assembly 140 and may assist in increasing clamping force of the threaded regions against the leadscrew 84.

The flanking bodies 162 may also include a pocket or receptacle 172. A channel 180 may extend from the receptacle 172 to the opposing side of the flanking body 162. The actuator 178 may be routed through this channel 180 and coupled to the retainer body 161. A housing 174 may surround the nut body 142 and actuation subassembly 158 when the nut assembly 140 is assembled. In certain examples, the housing 174 may be a multi-part housing which may be held together around the rest of the nut assembly 140 by one or more clasps 176 or fasteners. The housing 174 may include a fenestration 175 for each of the arms 164. This may allow for an increased displacement range of the arms 146.

Still referring to FIGS. 59A-E, when the nut assembly 140 is assembled, an end of the bias members 164 may be seated within a respective receptacle 172 defined on the flanking bodies 162. The opposing end of the bias members 164 may press against one of the flanges 160 defined in the base 144 of the nut body 142. There may be a small recess or pocket in the flange 160 to assist in locating the end of the bias member 164.

The actuation subassembly 158 may be translationally displaceable through a displacement range. At one end of the displacement range, the actuation subassembly 158 may be at its closest to the base 144 of the nut body 142. As best shown in FIG. 59A-B, the bias members 164 may urge the actuation subassembly 158 away from the nut body 142 toward the second end of the displacement range of the actuation subassembly 158.

An actuator 178 may be coupled to the actuation subassembly 158. When the actuator 178 is displaced, the actuation subassembly 158 may be driven to translationally displace with respect to the nut body 142. As the actuation subassembly 158 is displaced toward the base 144 of the nut body 142 by the actuator 178, the projections 152 may displace along their respective tracks 170. The path along which the tracks 170 extend may guide the projections 152 on each arm 146 of the nut body 142 to displace away from the axis of the nut assembly 140. In the example embodiment, this forces the arms 146 to splay apart from one another. This may displace the threaded terminal region 150 of each arm 146 out of contact with a leadscrew 84 of the drive assembly 82. Thus, the nut body 142 may be disengaged from the leadscrew 84. The projections 152 may be at a terminal end of their respective tracks 170 when the actuation subassembly 158 is displaced by the actuator 178 to the first end of its displacement range.

As force on the actuator 178 is relieved, the bias members 164 may urge the actuation subassembly 158 away from the base 144 of the nut body 142. This may in turn pull the arms 146 of the nut body 142 towards one another via the interaction of the projections 152 with the tracks 170. Thus, the threaded portion of the terminal regions 150 of the arms 146 may be forced into engagement with the leadscrew 84 by the bias members 164. The bias members 164 may also keep the arms 146 under a degree of tension buttressing them against buckling.

The projections 152 may be at an intermediate position in the tracks 170 when the threaded portion of the terminal region 150 of the arms 146 are urged into engagement with the leadscrew 84. This leads the bias members 164 to create a degree of clamping force which holds the threaded region of the arms 146 in engagement with the leadscrew 84. This may additionally help ensure that the arms 146 automatically be adjusted to maintain firm engagement with the leadscrew 84 in the event that any wear to the threading occurs over the usage life of the drive assembly 82. Thus the position of the actuation assembly 158 at the end of its displacement range opposite the base 144 may automatically alter slightly over time.

The nut assembly 140 may also be resistant to perturbations due to eccentricities in threading on the leadscrew 84. The arms 146 may distort and absorb eccentricities of the leadscrew 84. The slit 156 in each arm 146 may allow the associated region of the arm 146 to act as a living hinge based four-bar linkage. This may further bolster the ability of the nut assembly 140 to resist perturbation due to leadscrew 84 eccentricity. As mentioned elsewhere herein, this may increase flow rate accuracy over time.

Referring now primarily to FIG. 59D, the nut assembly 140 of FIGS. 59A-C is depicted installed in a drive head tube 36. As shown, the nut assembly 140 may be disposed coaxial with the leadscrew 84 and drive head tube 36. The housing 174 of the nut assembly 140 may include a terminal flange 173 with one or more notch 179 recessed therein. As shown, the drive head tube 36 may include respective protrusions 177 which extend into each notch 179. The protrusions 177 may inhibit rotational displacement of the nut assembly 140 relative to the drive head tube 36. The carriage 50 may couple around the terminal region of the drive head tube 36. The carriage 50 may include a receptacle 51 into which the terminal flange 173 of the housing 174 may mate. Thus, the terminal flange 173 of the housing 174 may be captured between the end of the drive head tube 36 and a portion of the carriage 50. This may inhibit the nut assembly 140 from translationally displacing relative to the drive head tube 36. Other nut assemblies 140 (see, e.g., FIG. 60A-D) may be retained in place in like manner.

Referring now to FIG. 60A-D, another exemplary nut assembly 140 is depicted. The example nut assembly 140 may include a set of arms 186. Each of the arms 186 may include a terminal region 188. The faces of the terminal regions 188 of the arms 186 facing the interior of the nut assembly 140 may be threaded so as to cooperate with a desired leadscrew 84 (e.g. any of those described elsewhere herein). Opposite the terminal region 188 of each arm 186 may be a pass-through 190. An intermediate region of each arm 186 may include a pivot bearing hole 191. The arms 186 may be coupled together via an arm bias member 224 such as a torsion spring or extension spring with legs that extend into the pass-throughs 190.

Example nut assemblies 140 may also include an actuation subassembly 192. The actuation subassembly 192 may include at least one bias member 194 and a main body 196. In the example embodiment, two bias members 194 which are depicted as compression springs are included. The main body 196 may include a base 198 from which a number of projections 200 or projecting bodies extend. The base 198 may include a central aperture 210 (best shown in FIG. 60B) which extends through the base 198. The central aperture 210 may include two opposing wall sections 212 which partially define the aperture 210 and are ramped or taper such that the width of the aperture 210 alters over its length. The ramped wall sections 212 may be angled with respect to the axis of the nut assembly 140. A first portion of the wall sections 212 nearest the projections 200 may be sloped at a sharper angle than a second portion of the wall sections 212 more distal to the projections 200. The wall sections 212 may be sloped such that the central aperture 210 has a smaller cross-sectional area at a region most distal the projections 200. The ramped wall sections 212 may form guide surfaces of the actuation subassembly 192.

Each of the projections 200 or projecting bodies may extend in a direction substantially parallel to the axis of the nut assembly 140. The projections 200 may include an interior face 202 with a trough 204. The trough 204 of each projection 200 may be disposed along the central region of the interior face 202 and extend across the entirety of the interior face 202 in a direction parallel to the axis of the nut assembly 140. The trough 204 may provide clearance to ensure that the leadscrew 84 is out of contact with the projections 200 when the nut assembly 140 is in an assembled state. The projections 200 may also each include a receptacle 206 (best shown in FIG. 60B). A passage 208 may extend from the receptacle 206 and into communication with notches 209 in the base 198. When the nut assembly 140 is assembled, the terminal regions 188 of the arms 186 may be disposed in a space intermediate the projecting bodies 200 and the projecting bodies 200 may be disposed in flanking relation to the arms 186.

The nut assembly 140 may further include a housing 214 which may form a stationary portion of the nut assembly 140. The housing 214 may be constructed of a number of housing portions 216A, B in certain examples. Each of the housing portions 216A, B may include a pivot bearing 222. When the nut assembly 140 is assembled, the pivot bearings 222 may extend through the pivot bearing holes 191 in the arms 186 allowing the arms 186 to rotationally displace about the axis of the pivot bearings 222. The housing portions 216A, B may be coupled together by a set of fasteners 220 (or in any other suitable manner). The fasteners 220 may extend through the pivot bearings 222 and into engagement with the opposing housing portion 216A, B.

When assembled, an actuator 178 may be coupled to the main body 196 of the nut assembly 140. When the actuator 178 is actuated, the main body 196 may be driven to translationally displace with respect to the housing 214 in a first direction. As the main body 196 is displaced into the arms 186 by the actuator 178, the angled wall sections 212 may collide with interface surfaces on respective arms 186, which in the example embodiment are provided on a face of each arm 186 opposite the threaded region. Further displacement of the main body 196 may cause the angled wall sections 212 to guide the arms 186 such that the arms 186 pivot toward the axis of the nut assembly 140 (as shown in FIG. 60B). This may drive the threaded regions of the arms 186 into engagement with the leadscrew 84. Thus, the nut assembly 140 may be actuated into engagement with the leadscrew 84. As the arms 186 are pivoted into engagement with the leadscrew 84, the arm bias member 224 may become distorted. Once force on the actuator 178 is relieved, the bias members 194 may urge the main body 196 in a second direction opposite the first direction. The arm bias member 224 may restore to a less distorted state and urge the arms 186 to pivot away from the leadscrew 84. Thus, the threaded portion of the arms 186 may be disengaged form the leadscrew 84.

When the arms 186 are cinched into engagement with the leadscrew 84, there may be an additional span of the tapered wall sections 212 beyond that which is in contact with the arms 186. This may help ensure that the arms 186 automatically be adjusted to maintain firm engagement with the leadscrew 84 in the event that any wear to the threading occurs over the usage life of the drive assembly 82. Thus the position of the main body 196 when the arms 186 are in engagement with the leadscrew 84 may automatically alter slightly over time and the extra span of tapered wall sections 212 may ensure the arms 186 are appropriately cinched into contact with the leadscrew 84. The nut assembly 140 may also be resistant to perturbations due to eccentricities in threading on the leadscrew 84. The arms 146 may distort and absorb eccentricities of the leadscrew 84. As mentioned elsewhere herein, this may increase flow rate accuracy over time.

Referring now to FIG. 61 a view of an example drive head 38 which may be used in example syringe pumps 10 including nuts assemblies 140 of the type shown in FIGS. 59A-60D is depicted. A portion of the housing 250 of the drive head is removed to illustrate the mechanical effects of rotation of a dial 9505. As shown in FIG. 61, the dial 9505 is coupled to a shaft 9547, a cam 9548, and an actuator driver 9554 which may operate an actuator 178 for an example nut assembly 140. A spring 9557 is operatively coupled to the shaft 9547 to bias the dial 9505 and the shaft to rotate toward a closed position (as shown in FIG. 61). A gear 9553 is operatively coupled to a potentiometer 9559. The potentiometer 9559 is coupled to a circuit board 9558 which is configured to provide a controller 15 with the rotational position of the gear 9553 (described below).

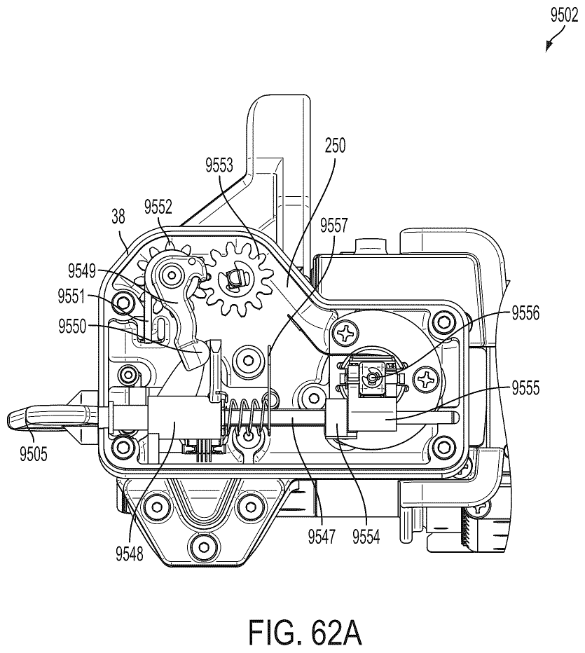

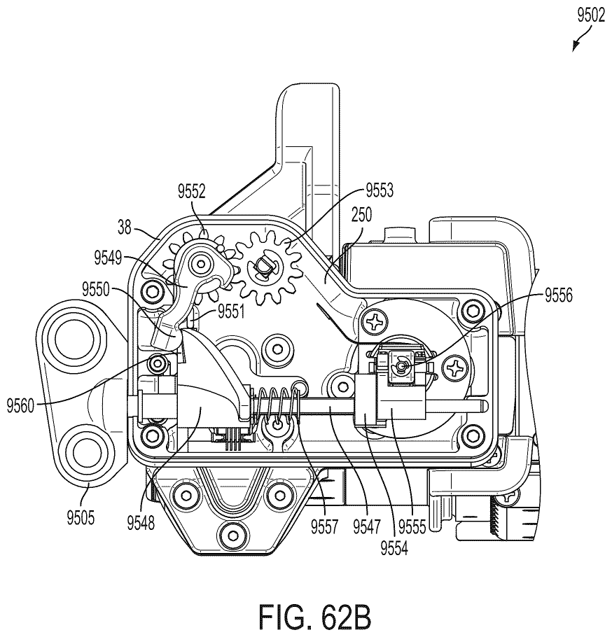

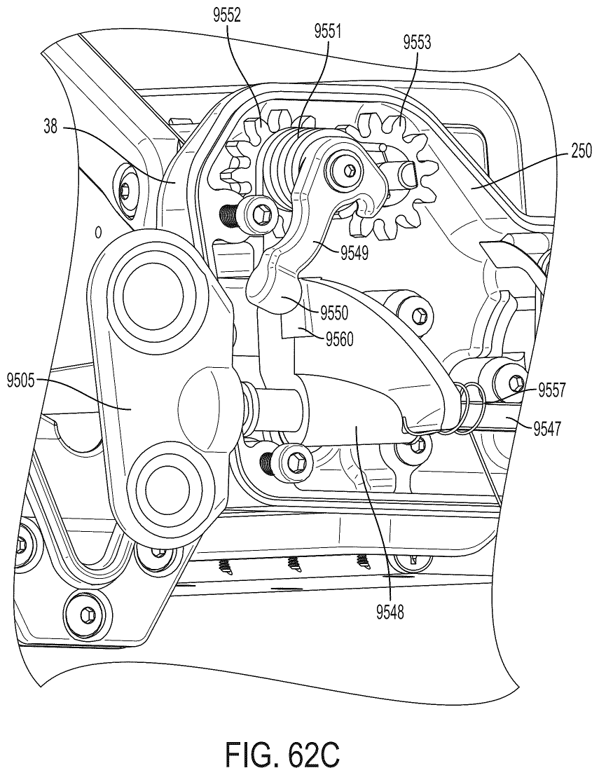

Referring now to FIGS. 62A-C, the circuit board 9558 and the potentiometer 9559 have been removed to aid in viewing the internal parts of the drive head 38. That is, FIGS. 62A-C show several views of the drive head 38 with a portion of the housing 250 and a circuit board 9558 removed to illustrate the mechanical effects of rotation of the dial 9505. As shown in FIG. 62A, the dial 9505 is coupled to the cam 9548 such that rotation of the dial 9505 into an open position causes the cam 9548 to rotate such that the rocker arm 9549 rotates as a cam follower 9550 of the rocker arm 9549 engages with the cam 9548. The rocker arm 9549 is coupled to a gear 9552. A gear 9553 is coupled to the gear 9552 that is coupled to the rocker arm 9549. The gear 9552 and rocker arm 9549 are coupled to a spring 9551 such that the rocker arm 9549 is biased such that the cam follower 9550 is biased toward the cam 9548. FIG. 62B shows the configuration in which the dial 9505 is in the fully open position. Note that the rocker arm 9549 has rotated from its position in FIG. 62A, and note also that the gear 9553 has rotated by a corresponding amount. The gear 9552 may be coupled to a plunger flange grasper body 290A, B and the gear 9553 is coupled to another plunger flange grasper body 290A, B (see, e.g., FIG. 25).

When the dial 9505 has been turned to a fully open position, the cam 9548 engages into a detent 9560 of the cam 9548. FIG. 62C shows a close-up view to illustrate the detent 9560. As is easily seen in FIG. 62C, the cam follower 9550 may fit into the detent 9560, which holds the dial 9505 in a “dwell” position. That is, although a user may remove their hand from the dial 9505, the dial 9505 remains in the fully open position as shown in FIG. 62C. In some embodiments, the spring 9557 does not provide enough torque on the shaft 9547 to overcome the detent 9560 without user assistance.

When the dial 9505 is turned from the open position as in FIG. 62B back to the closed position, the plunger flange grasper bodies 290A, B (see, e.g. FIG. 25) will rotate toward a flange 28 of a plunger 24 of a syringe 18. The plunger flange grasper bodies 290A, B will stop rotating toward each other when they contact the flange 28 of the plunger 24. This will cause the cam follower 9550 to leave the cam 9548 because the surface of the cam 9548 will continue to move away from the cam follower 9550. The rocker arm 9549 is unable to rotate further because it is coupled to one of the plunger flange grasper bodies 290A, B whose movement is constrained by the flange 28 of the plunger 24 of the syringe 18. The position of the plunger flange grasper bodies 290A, B may be determined by one or more potentiometer(s) 9559 and communicated to a controller 15 of the syringe pump 10. The controller 15 may use this position to estimate a size characteristic of the syringe 18. That is, the position of the plunger flange grasper bodies 290A, B when grasping around the flange 28 of the plunger 24 of the syringe 18 may be input parameters into a syringe database to determine which syringe model number is loaded to determine the internal diameter of the syringe 18. The controller 15 of the syringe pump 10 may use the identified syringe 18 to set the internal diameter value for delivery volume calculations and/or internal syringe pressure estimation purposes.

In syringe pump 10 embodiments including a nut assembly 140 such as those described in relation to FIGS. FIGS. 59A-60D, the actuator 178 for the nut assembly 140 may include a set of rigid wires. Depending on the nut assembly 140, the actuators may extend through the channels 180 in the flanking bodies 162 (see, e.g., FIG. 59A) or passages 208 in the projections 200 (see, e.g. FIG. 60C). The example wires may have ends which are anchored to the retainer body 161 of the actuation subassembly 158 (see, e.g., FIG. 59A) or notches 209 in the base 198 of the actuation subassembly 192 (see, e.g., FIG. 60C). In the embodiment shown in FIGS. 59A-D, the flanges 160 in the base 144 of the nut body 142 may include notches 182 to accommodate such wire actuators 178.

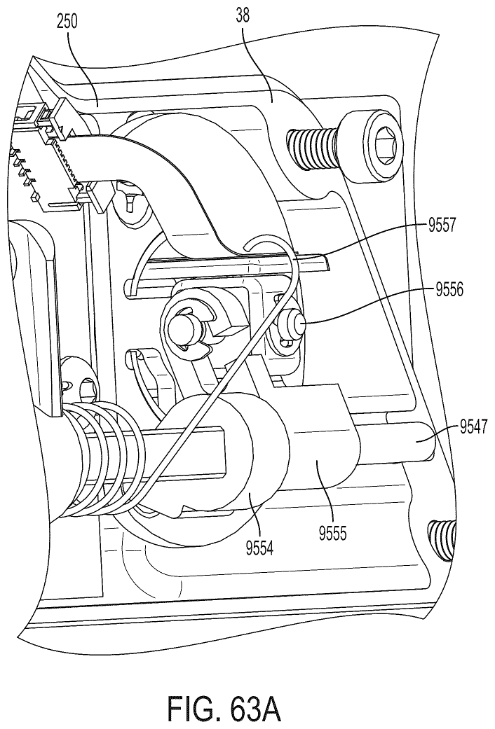

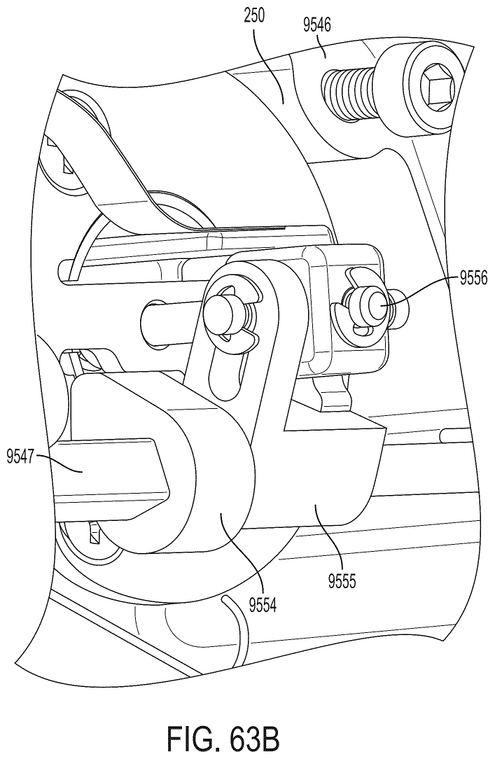

With reference now primarily to FIGS. 63A-B two close-up views of the inner cavity of the example drive head 38 shown in FIG. 61 are depicted. As the shaft 9547 is rotated, the actuator driver 9554 rotates. When the dial 9505 (see FIG. 61) is near the fully open position, the actuator driver 9554 engages the link 9555 to displace the actuator 178 as shown in FIG. 63B for example.

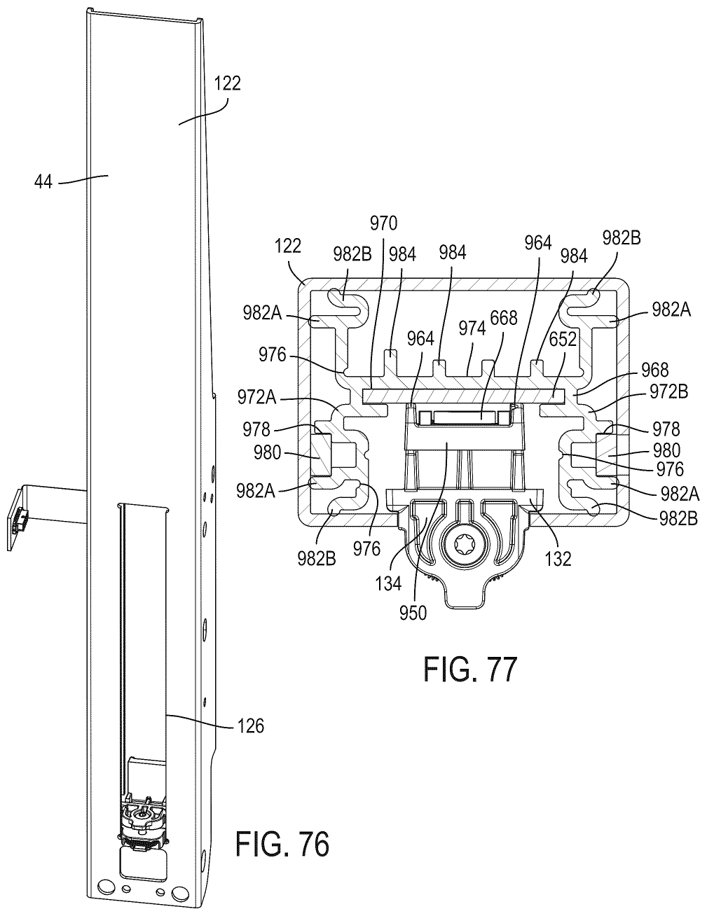

Referring now to FIGS. 64A-C, a number of views of an exemplary drive assembly 82 and support assembly 120 are depicted. Though a drive assembly 82 similar to that shown in FIG. 44A is depicted, any drive assemblies 82 described herein may be paired with a support assembly 120 like that shown in FIGS. 64A-C. As shown, the support assembly 120 may include a rigid body such as an elongate member or column 122 which defines a bearing surface 44. The support assembly 120 may extend along the length of the main housing 12 of a syringe pump 10 and at least to the extreme end of the displacement range of the drive head 38 of the syringe pump 10. As indicated elsewhere herein, the handle 34 for the syringe pump 10 may be attached to or built into the support assembly 120. In the example embodiment, the rigid body 122 is constructed of piece of sheet metal. In other examples, the rigid body 122 may be formed from an extruded metal body (see, e.g., FIGS. 76-77). Other rigid materials may also be used. The example drive head 38 includes a bushing portion 124 recessed into the face of the drive head 38 most proximate the rigid body 120. The bushing portion 124 may accept an adjacent segment of the bearing surface 44.

The rigid body 122 may include a channel 126 which may be cut into a portion of the rigid body 122 internal to the main housing 12. The channel 126 may be present in the portion of the rigid body 122 traversed by a carriage 50 coupled to the end of the drive head tube 36 opposite the drive head 38. The carriage 50 may include a guide assembly 128 and the portion of the rigid body 122 including the channel 126 may act as a track for the guide assembly 128 guiding and supporting the carriage 50 as the carriage 50 is displaced. The guide assembly 128 may have a first portion 130 disposed on a first side of the rigid body 122 and a second portion 132 disposed on an opposing side of the rigid body 122. The first and second portions 130, 132 may overhang portions of the rigid body 122 on opposing sides of the channel 126. The first and second portion 130, 132 may be coupled together by a span 134 of material which extends through the channel 126. Thus, the guide assembly 128 may have a cross-sectional shape approximating the Latin character “I” and behave as a bearing which is guided by the rigid body 122. The span 134 may be integrally formed with part of either the first or second portion 130, 132 in certain examples. As mentioned in relation to FIGS. 38A-39B, this may help bestow a high degree of stiffness to the drive assembly 82 which may be beneficial for a number of reasons discussed elsewhere herein.

Referring now to FIGS. 65A-67B, and FIGS. 73A-H, exemplary embodiments of linear position sensing assemblies 650 are depicted. The linear position sensing assembly 650 embodiments depicted may be used for various purposes within a syringe pump 10. For example, the linear position sensing assemblies 650 may be used to sense the position of the carriage 50 within the drive assembly 82. Linear position sensing assemblies 650 described herein may also be used to sense the translational position of other components included in a syringe pump 10. For example, linear position sensing assemblies 650 shown herein may be used to sense the position of a syringe barrel clamp assembly 484 (see, e.g. FIG. 14). Of course, linear position sensing assemblies 650 described herein may be used in a wide range of other applications (e.g. robotic surgery, additive manufacturing processes such as 3D printing, 3D bioprinting, etc.). Linear position sensing assemblies 650 described herein may be particularly well suited to applications where very high positional resolution is desired. The linear position sensing assemblies 650 shown herein are merely depicted for example. Other varieties of position sensing assemblies may be utilized in alternative syringe pump 10 embodiments. For example, linear potentiometers may be used. Alternatively or additionally, translational position data may be collected from other or additional sources. For example, data from a motor encoder 85A may be used in conjunction with data from a linear position sensing assembly 650 monitoring the carriage 50 position to determine position of the carriage 50. This may add redundancy and facilitate generation of alerts in the event a controller 15 determines data from different sensors are in disagreement beyond some threshold amount.

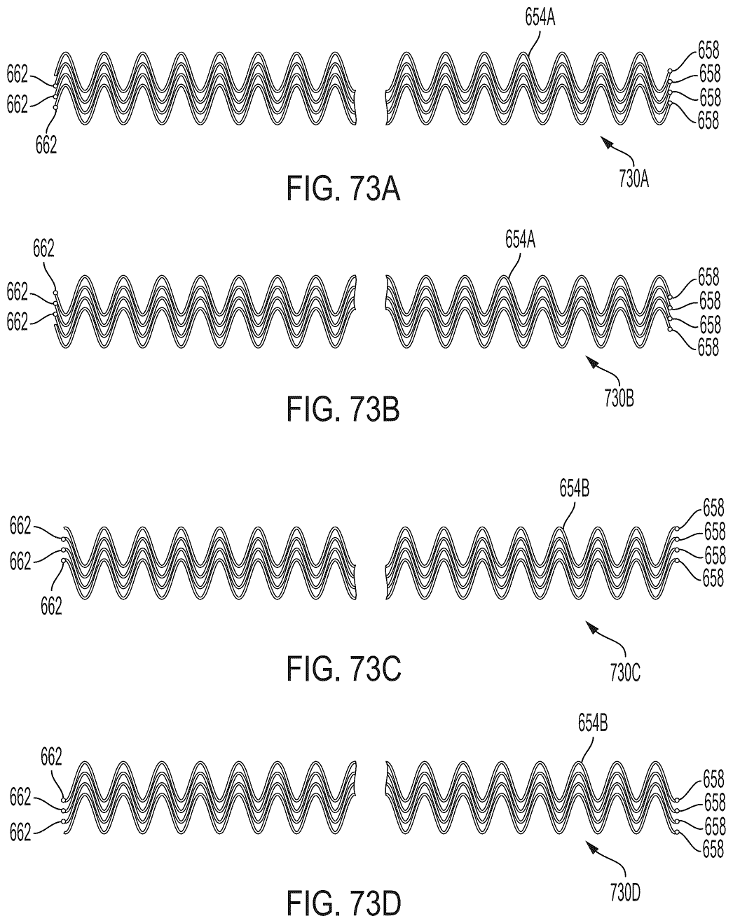

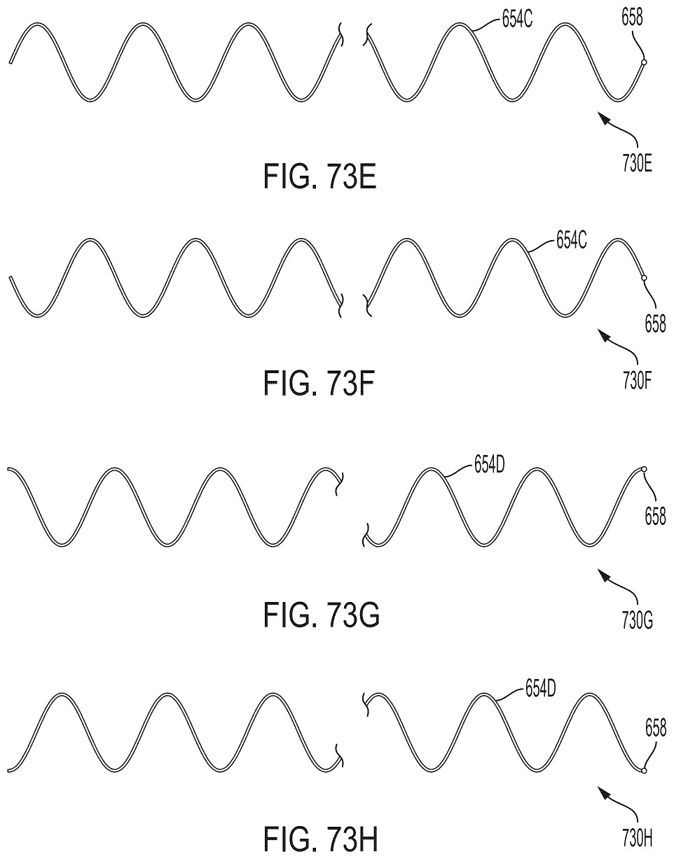

Referring primarily to FIGS. 65A-65B, a top and bottom plan views of an example linear position sensor assembly 650 are respectively depicted. A linear position sensing assembly 650 may include a PCB 652 with a plurality of sensing traces 654A-D. The sensing traces 654A-D may be provided on the PCB 652 in sinusoidal patterns. The PCB 652 may be a multiple layer PCB 652 with sensing trace 654A-D portions on each layer of the PCB 652. Only a portion of sensing trace 654A is visible in FIG. 65A. The entirety of the sensing traces 654A-D for the exemplary PCB 652 are depicted in FIGS. 73A-H. Each sensing trace 654A-D may be begin on an originating layer of the PCB 652 and extend from a trace origination region 656 to a via 658 in a layer transition region 660 of the PCB 652. Each sensing trace 654A-D may return or double back, on another layer of the PCB 652, toward the trace originating region 656. The portion of a sensing trace 654A-B on the respective originating layer may be arranged to be 180° out of phase with the portion of the sensing trace 654A-D on the return layer of the PCB 652 for that sensing trace 654A-D (best shown in FIGS. 73A-H). In certain examples, each sensing trace 654A-D may pass through a return via 662 in the trace originating region 656 an additional trace portion on the respective originating layer may extend from the return via 662 to another via 658 in the layer transition region 660. The sensing trace 654A-D may continue on the return layer toward the trace originating region 656. Thus each sensing trace 654A-D may double back multiple times on the PCB 652. Each sensing trace 654A-D may include, for example, four or eight, trace portions on the trace originating layer and return layer. The return layer may, though need not, be an adjacent layer of the PCB 652. Certain sensing traces 654A-D may double back a different number of times than other sensing traces 654A-D. The vias 658 in the originating region 656 and layer transition region 660 may be outside of the sensed region of the linear position sensor 650.

Referring primarily to FIG. 65A in conjunction with FIGS. 73A-H, at least one sensing trace 654A, C may be a sine wave trace and at least one other sensing trace 654B, D may be a cosine trace. In certain examples, multiple sine wave sensing traces 654A, C and multiple cosine wave sensing traces 654B, D may be included. The periods of each of the sine wave sensing traces 654A, C and each of the cosine wave sensing traces 654B, D may differ. In some embodiments, there may for example be short period sine and cosine wave sensing traces 654A, B. The short period sensing traces 654A, B may have periods of 0.1 inch or less. In certain implementations, the short period sensing traces 654A, B may have periods of 2 mm or less. The PCB 652 may also include at least one sine wave sensing trace 654C and at least one cosine wave sensing trace 654D that have relative long periods compared to those of the short period sensing traces 654A, B. In certain embodiments, the long period sensing traces 654C, D may have periods substantially equal to the distance between the trace originating region 656 and layer transition region 660 of the PCB 652. Pairs of long and short period sine wave sensing traces 654A, C and pairs of long and short period cosine wave sensing traces 654B, D may be included with periods of co-prime lengths. In other examples, sensing traces 654A-D may be arrayed in a Nonius pattern.

As shown, the PCB 652 may also include an excitation coil 664. The excitation coil 664 may surround the sensing traces 654A-D included on the PCB 652 and may, though need not necessarily be disposed on an exterior layer of the PCB 652. In other embodiments, traces for the excitation coil 664 may be provided on multiple layer of the PCB 652. The excitation coil 664 may generally be routed to form an obround shape with substantially straight regions extending adjacent the extent of the sensing traces 654A-D. The excitation coil 664 may be powered by an oscillator at a desired frequency (e.g. about 187 kHz).

Referring now primarily to FIG. 65B, a bottom plan view of the linear position sensing assembly 650 is depicted. As shown, ferrite bodies 666A-C (e.g. strips of ferrite material) may be associated with various regions on the side of the PCB 652 opposite the excitation coil 664. One ferrite body 666A may be positioned in alignment with the location of the sensing traces 654A-D included on the PCB 654. Ferrite bodies 666B, C may also be positioned in alignment with the regions of the excitation coil 664 adjacent the sensing traces 654A-D. The ferrite bodies 666A-C will be further described later in the specification.

As shown, a middle portion of the linear position sensing assembly 650 has been removed in FIGS. 65A-B to truncate the linear position sensing assembly 650 for case of illustration. The sensing traces 654A-D in FIGS. 73A-H have been depicted in a similar manner. The distance between the trace origination region 656 and layer transition region 660 and the distance spanned by the sensing traces 654A-D on each layer may differ depending on the embodiment. This distance may be selected based on the desired displacement range of a target assembly 668 which is to be sensed by the linear position sensing assembly 650. In certain examples where the linear position sensing assembly 650 is monitoring the position of a carriage 50 (see, e.g., FIGS. 64A-C) of a syringe pump 10 drive assembly 82, the distance may be at least 140 mm. In alternative examples, the target assembly 668 may be stationary and the PCB 652 may displace in tandem with a displaceable component of interest (e.g. portion of a drive assembly 82 such as a carriage 50).

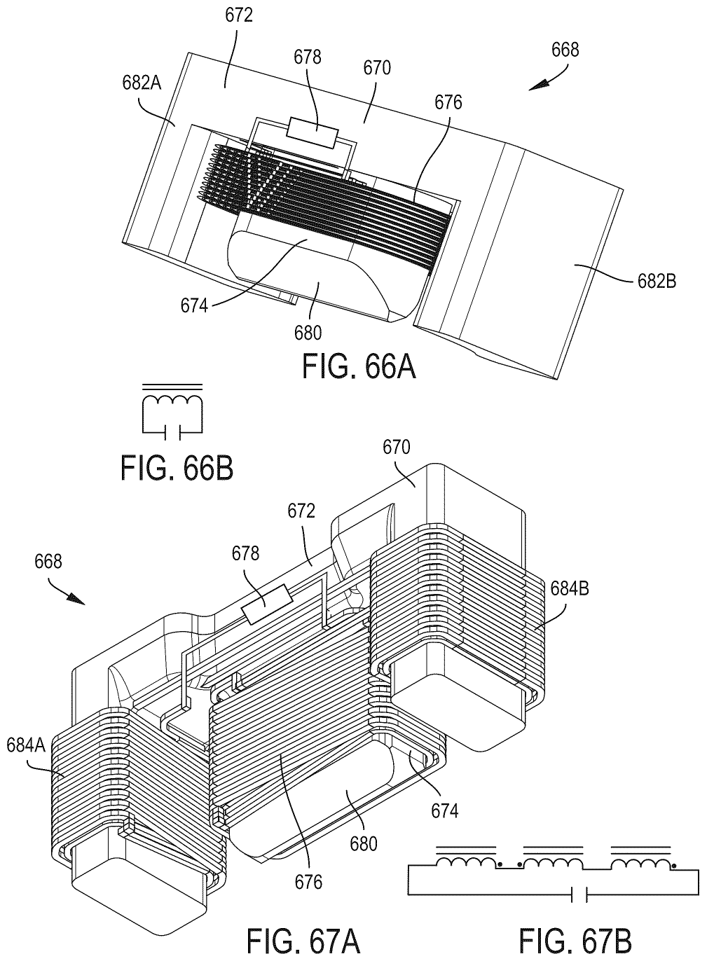

Referring now to FIG. 66A in addition to FIGS. 65A-B, the linear position sensing assembly 650 may also include a target assembly 668. The target assembly 668 is schematically depicted in FIG. 66B. A target assembly 668 may be displaced in tandem with a component of interest included in the syringe pump 10 such as the carriage 50 (see, e.g., FIGS. 64A-C) of the drive assembly 82. In such examples, as fluid is dispensed out of a syringe 18 installed in the syringe pump 10, data from the linear position sensing assembly 650 may allow for a controller 15 to determine the location of the carriage 50. In turn, the controller 15 may orchestrate dispensing of fluid from a syringe 18 with commands to the drive motor 92 (see, e.g., FIG. 58B) that are based at least in part on data from the linear position sensing assembly 650. Position data from a linear position sensing assembly 650 may also be utilized to determine the amount of volume dispensed from a given syringe 18 over a period of time (e.g. based on the identity of the syringe 18 and position data at different time points). In other examples where, for example, a linear position sensing assembly 650 is used to monitor the position of a barrel clamp assembly 484 (see, e.g., FIGS. 14-15), the target assembly 668 may be provided on a translating part of the barrel clamp assembly 484. The position of the target assembly 668 may allow for a controller 15 to determine whether a syringe 18 is clamped (or properly clamped) by the barrel clamp assembly 484. Additionally, the position of the target assembly 668 may allow for identification of the syringe 18 being clamped by providing information a controller 15 may analyze to determine the diameter of the syringe 18.

The example target assembly 668 depicted in FIG. 66A, includes a main body 670. The main body 670 in FIG. 66A is generally provided in the shape of the Latin character “E”. The main body 670 may be a ferrite material. The specific material may be chosen to have a high magnetic permeability (e.g. 250-300μ) at a desired frequency (e.g. 187 kHz). The main body 670 may include a back bone portion 672. A central peg 674 may extend from the back bone 672 at an angle substantially perpendicular thereto and may be surrounded by a coil 676 which may be constructed of fine wire. For example, the wire may have an American Wire Gauge size of 35-44 (e.g. 40 AWG) and be turned 300-400 (e.g. 350) times to form the coil. A capacitor 678 may be included parallel to the coil 676 to form a resonant circuit and may have a capacitance of 80-120 pF in certain examples. The target assembly 668 may have an inductance of less than 3 mH.

Lateral to the central peg 674 may be arms 682A, B on each side of the peg 674. The arms 682A, B may extend from opposing end regions of the back bone 672 in the same direction as the central peg 674. These arms 682A, B may generally align over the regions of excitation coil 664 which extend adjacent to the sensing traces 654A-D on the PCB 652. The arms 682A, B may have a length slightly longer than that of the central peg 674. The surface of the arms 682A, B most distal to the back bone portion 672 may be spaced from the excitation coil 664 by a distance of less than 0.01 inches (e.g. 0.005 inches) in certain examples. In some examples, the ends of the arms 682A, B most distal the back bone portion 672 may ride along a spacer (e.g. polycarbonate spacer) interposed between the target assembly 668 and the PCB 652.

Referring now to FIG. 67A, another example target assembly 668 is depicted. The target assembly 668 of FIG. 67A is schematically depicted in FIG. 67B. In certain examples, the arms 682A, B lateral to the central peg 674 may also be surrounded by coils 684A, B which may help limit efficiency loses. These coils 684A, B may be 180° out of phase with the coil 676 disposed on the central peg 674.

In the example embodiments shown, the tip region 680 of the peg 674 most distal to the back bone 672 may be beveled such that the peg 674 decreases in width as distance from the back bone 672 increases. In certain examples, the tip region 680 may be a wedge shape which tapers to a width of 0.015″ or less. The tip region 680 may have a width which is 10-30% (e.g. 25%) the period of the length of the shortest period traces on the PCB 652. Values less than 10% period of the length of the shortest period traces are also possible. When installed in the linear position sensing assembly 650, the end of the tip region 680 most distal to the back bone 672 may be positioned over the sensing traces 654A-D of the PCB 652. The end of the tip region 680 may be spaced from the PCB 652 by a distance of less than 0.01 inches (e.g. 0.005 inches) in certain examples. In some examples, the tip region 680 may ride along a spacer (e.g. polycarbonate spacer) interposed between the target assembly 668 and the PCB 652. Where a spacer is included for the arms 682A, B and/or tip region 680, the spacer may assist in maintain a constant distance between portions of the target assembly 668 and the PCB 652.

The bevel geometry of the end of the central peg 674 may assist in facilitating high resolution positional sensing (certain example linear position sensing assemblies 650 may be capable of submicron resolution). As mentioned above, the rear of the PCB 652 may be provided with a ferrite backing (see, e.g., ferrite body 666A of FIG. 65B) positioned in alignment with the sensing traces 654A-D. This may direct the magnetic flux leaving the tip of the central peg 674 through the PCB 652 and help confine the magnetic field generated in this region while the target assembly 668 is resonating such that it is concentrated in a relatively thin area (e.g. a width significantly less than the period of the short period sensing traces 654A-D). In some embodiments, the width of the B-field may be 10% or less the length of the period for the shortest period traces on the PCB 652. Ferrite bodies 666B, C associated with the excitation coil 664 may help limit direct leakage between the excitation coil 664 and sensing traces 654A-D. The ferrite material associated with the excitation coil 664 may also augment coupling between the excitation coil 664 and the target assembly 668. The ferrite bodies 666A-C may not be directly attached to the PCB 652 and may be disposed in proximity to the PCB 652 and in alignment with the sensing traces 655A-D and excitation coil 664 portions as mentioned above.

In use, the excitation coil 664 may be used to illuminate the target assembly 668 with a changing magnetic field. The frequency at which the excitation coil 664 is powered may be substantially a resonant frequency of the resonant circuit formed in the target assembly 668. After illuminating the target assembly 668 with the changing magnetic field, the excitation coil 664 may be turned off. Illumination of the target with the magnetic field may generate electromagnetic oscillation in the target assembly 668 resonant circuit which will continue after the excitation coil 664 is no longer powered. In turn, this may generate an EMF in the sensing traces 654A-D defined in the PCB 652. A characteristic of interest of the generated EMF in each sensing trace 654A-D may vary depending on the position of the target assembly 668 over the respective sensing trace 654A-D. This condition of interest may be monitored to collect position data for analysis by a controller 15. In certain examples, the generated voltage in the sensing traces 654A-D may vary depending upon the location of the target assembly 668 over the sensing traces 654A-D and this induced voltage may be used to determine positional information about the target assembly 668.

By having sensing traces 654A-D with different length periods, it is possible to determine where along the PCB 652 the target assembly 668 is located. A controller 15 may receive and analyze the induced voltage output from, for example, the short period sensing traces 654A, B and long period sensing traces 654C, D. The induced voltage values from the long period sensing traces 654C, D may be used to determine what period in a short period sensing trace the target assembly 668 is positioned over. The induced voltage in that the short period sensing trace 654A-D may then yield a very high resolution position for the target assembly (e.g. a 16 bit resolution over the length of the short periods). In some embodiments, the induced voltages may be fed through one or more amplifier before reaching the analog to digital converter. Filtering (e.g. bandpass filtering) may be applied as well. During calibration a look-up table may be generated which may be used to determine position of the target assembly 668 based on the induced voltages collected from the sensing traces 654A-D. This look-up table may be referenced by the controller 15 when data is received from a linear position sensing assembly 650 to determine the location of target assembly 668 and carriage 50 (or other component of the syringe pump 10) to which the target assembly 668 is coupled.

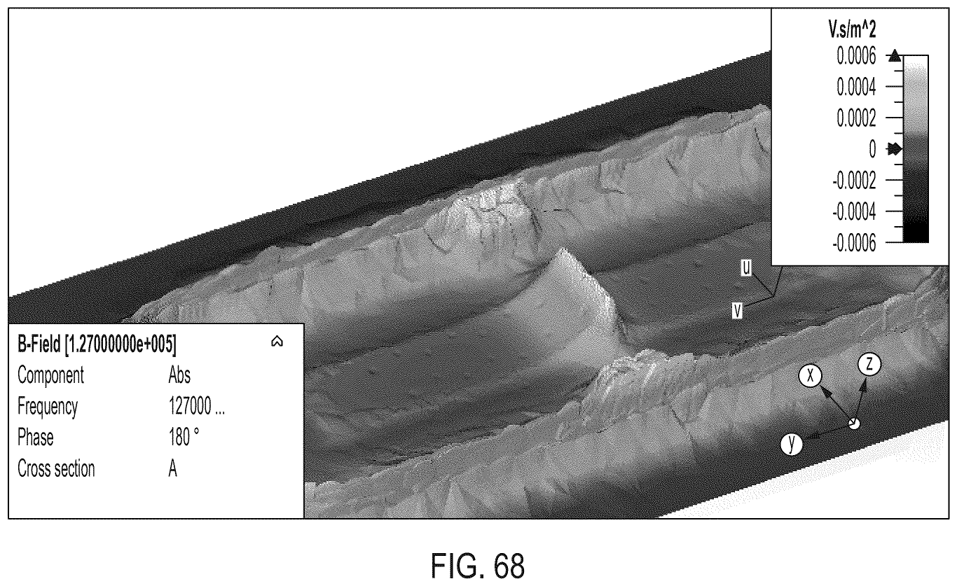

Referring now to FIG. 68, a simulated extruded 2D field strength coupling map 690 for a portion of a linear position sensing assembly 650 including a target assembly 668 of the variety shown in FIG. 67A is depicted. The field strength shown is that simulated to be present in a cut plane between the top of an example PCB 652 and the end of the tip region 680 of a central peg 674 of a target assembly 668. The field strength map 690 is generated from a portion of the resonant cycle of the target assembly 668 where the field strength in the region of the sensing traces 654A-D on the PCB 652 is at its strongest. As shown, the bevel geometry of the tip region 680 of the example target assembly 668 in conjunction with the ferrite body 666A on the PCB 652 may produce a magnetic field which is concentrated in a thin region. This may facilitate use of short period sensing traces 654A, B which have particularly short periods (e.g. as short as 2 mm or less).

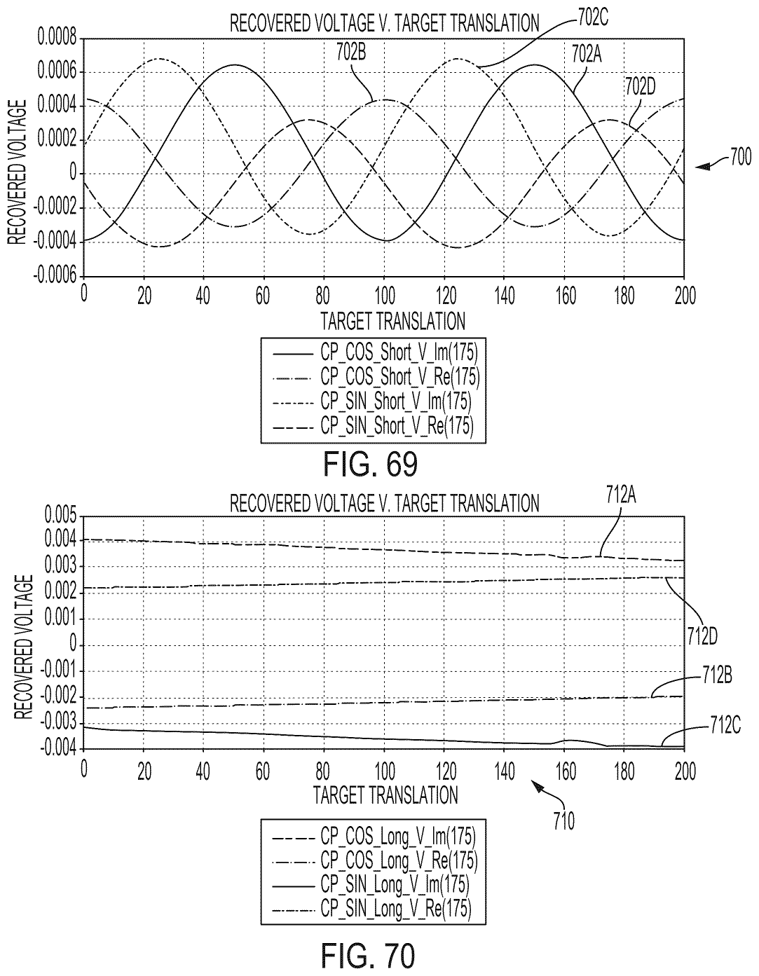

FIG. 69 and FIG. 70 respectively depict example plots 700, 710 of simulated induced voltages 702A-D, 712A-D in exemplary short period sensing traces 654A, B and long period sensing traces 654C, D generated with displacement of a target assembly 668 over a section of these traces 654A-D. The long period sensing traces 654C, D from which the simulated induced voltages were plotted had period lengths substantially equal to the distance between trace originating regions 656 and the layer transition region 660 on the PCB 652. As mentioned above, during a calibration procedure, the induced voltages 702A-D, 712A-D may be collected over known translational displacement of a target assembly 668 to generate a look-up table that may be referenced by a controller 15 to resolve the position of the target assembly 668. Though the long period traces 654C, D used to generate plot 710 had a single period per PCB layer, other long period trace 654C, D patterns may be used. Any length periods may be used to form the sensing traces 654A-D so long as a comparison of the sensed characteristics of interest off the sensing traces 654A-D would not generate a position result open to multiple interpretations. For example, period lengths of the short sensing traces 654A, B and long sensing traces 654C, D may be co-prime or may be provided in a Nonius pattern.

In certain embodiments, voltage readings may be fed into a function or formula solved by the controller 15. The following function may be used to return a value in degrees for data from the short period sensing traces 654A, B and long period sensing traces 654C, D:

ATn2InDegrees((COS_volts_real*(Sin((COS_volts_imaginary+90)*Pi/180))),(SIN_volts_real*(Sin((SIN_volts_imaginary+90)*Pi/180))))

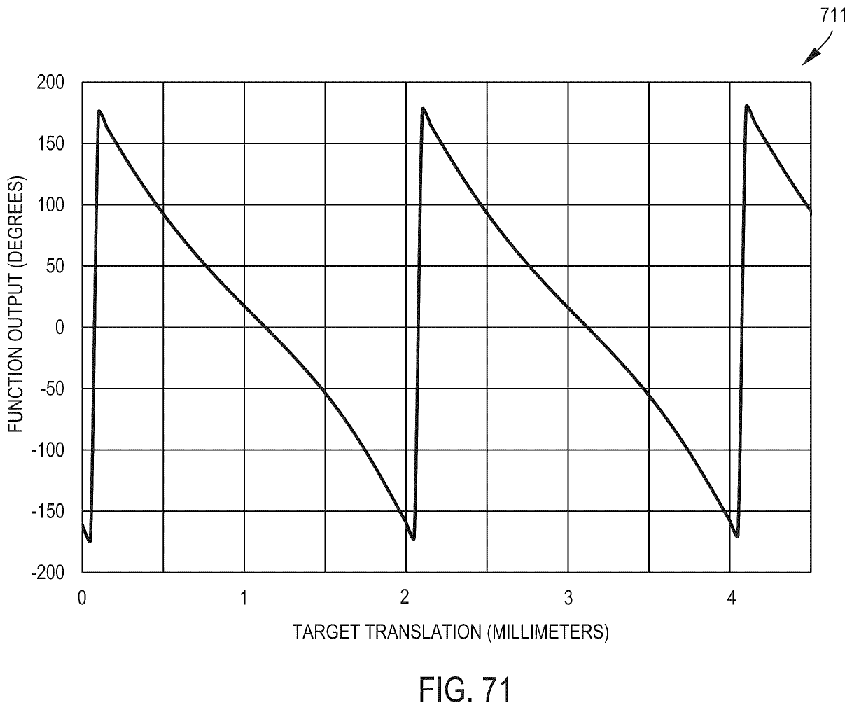

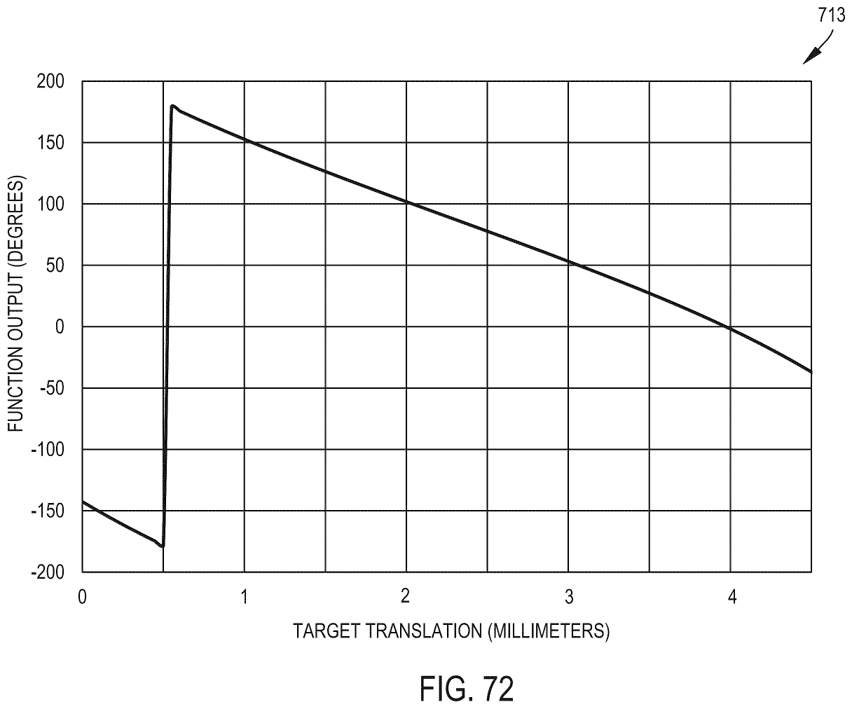

Example plots 711, 713 showing the output of this function over displacement of a target assembly 668 are depicted in FIGS. 71-72. The plot 711 of FIG. 71 was generated from simulated data from CST Studio Suite electromagnetic simulation software using example short period sensing traces 654A, B. The plot 713 of FIG. 72 was generated from simulated data from CST Studio Suite electromagnetic simulation software using example long period sensing traces 654C, D.

Referring now to FIGS. 73A-73H, a number of views of various layers 730A-H of the PCB 652 are depicted. The layers 730A-D depicted in FIGS. 73A-D include short period sensing traces 654A, B of the exemplary PCB 652. The layers 730E-H depicted in FIGS. 73E-H include long period sensing traces 654C, D of the example PCB 652. A middle portion of each layer 730A-H has been removed to truncate the sensing traces 654A-D for case of illustration. The distance traversed by sensing trace 654A-D portions on each layer 730A-H may vary by embodiment depending on the displacement range over which sensing of the target assembly 668 is desired. In the example embodiment, each short period sensing trace 654A, B doubles back four times. The long period sensing traces 654C, D double back a single time. In other embodiments, each sensing trace 654A-D may double back only a single time or all sensing traces 654A-D may double back a plurality of times (e.g. the same plurality of times in all traces 654A-D). In certain examples, each sensing trace 654A-D or at least the short period sensing traces 654A, B may double back eight or more times. An increase in the number of times a sensing trace 654A-D doubles back may increase the induced voltage on that sensing trace 654A-D while the target assembly 668 resonate. This may be due to an increase in composite voltage for each additional doubling back in series. As shown each sensing trace 654A-D portion on a given layer may be offset from adjacent portions of that sensing trace 654A-D by an even amount. The sine sensing traces 654A, C and cosine sensing traces 654B-D are presented in pairs in the drawings, however, this is for convenience. The layers 730A-H on which a particular sensing trace 654A-D are defined need not be adjacent layers within the PCB 652. The amplitude of the short period sensing traces 654A, B and the long period sensing traces 654C, D are substantially the same though need not be so in all examples. The amplitude of each sensing trace 654A-D may be in a range of 0.15-0.25 inches (e.g. 0.2 inches). The amplitude of each sensing trace may be 75-100% the width of the magnetic field generated by the target assembly 668 as the target assembly 668 resonates. Increasing the amplitude may assist in increasing the induced voltage in the sensing traces 654A-D.

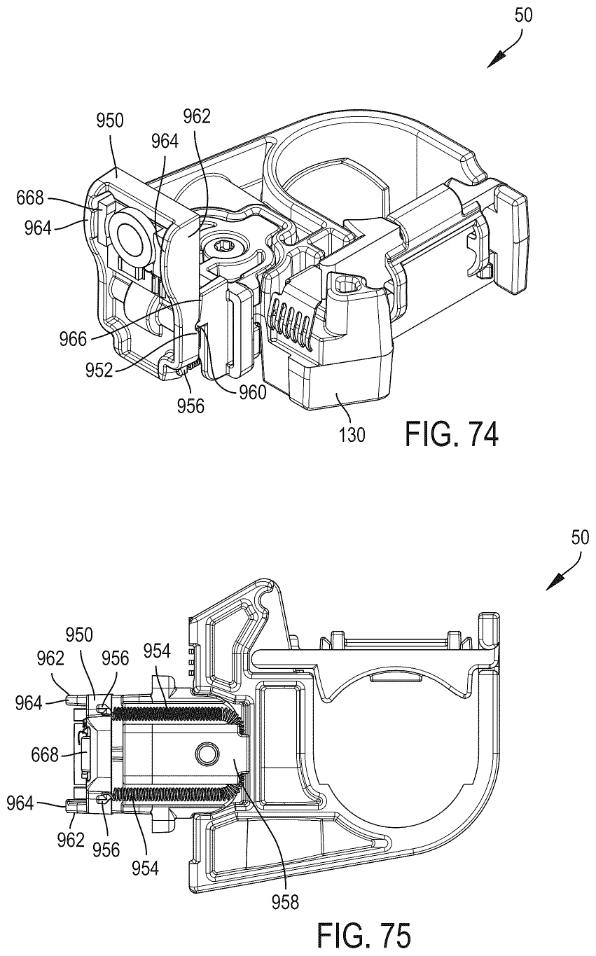

Referring now to FIGS. 74-75, views of an example embodiment of a carriage 50 to which a target assembly 668 is coupled are depicted. As shown, the target assembly 668 may be mounted to a paddle 950. The paddle 950 may be coupled to a second portion 132 of the guide assembly 128 of the carriage 50, but moveable over a displacement range relative to the second portion 132. The paddle 950 may be held in place on the carriage 50 by one or more bias member 954. The one or more bias member 954 may be coupled to the paddle 950 and urge the paddle 950 as well as the carriage 50 and may urge the paddle 950 toward one extreme of the displacement range. The carriage 50 may include one or more stop surface 966 which may block displacement of the paddle 950 beyond a certain point. Thus, the displacement range of the paddle body 950 may be defined at least in part by the one or more stop surface 966 on the carriage 50.

In the example, the paddle 950 may include a rocker surface 952 defined on a surface of the paddle 950. The target assembly 668 may be mounted to a surface of the paddle 950 opposite the rocker surface 952. The rocker surface 952 may be a rounded depression in the paddle body 950. The one or more bias member 954 may hold the rocker surface 952 of the paddle 950 against a rocker projection 960 defined on the carriage 50. In such examples, the paddle 950 may rock about the rocker projection 960 through its displacement range relative to the second portion 132 of the guide assembly 128.

As shown, the paddle 950 may include anchor points 956. In addition to being coupled to the carriage 50, the one or more bias member 954 may be coupled to the paddle 950 at the anchor points 956. In the example embodiment, the carriage 50 includes a hook 958 and the one or more bias member 954 is depicted as an extension spring. The ends of the extension spring are placed over the anchor points 956 and the center region of the extension spring is captured by the hook 958.

Flanking the target assembly 668, the paddle 950 may include raised sidewalls 962. The sidewalls 962 may substantially mirror one another and include a contact face 964 which follows a curved path giving the sidewalls 962 a height which varies along the extent of the sidewalls 962. The paddle 950 may be constructed of a non-metallic material such as a plastic.

Referring now also to FIG. 76-77, a portion of the carriage 50 and rigid body 122 of the support structure 120 (see, e.g., FIGS. 64A-C) of a syringe pump 10 are shown. As shown, a linear position sensor PCB 652 (such as those of any linear position sensor 650 embodiments described herein) is depicted in the rigid body 122. When a syringe pump 10 is assembled, the contact surfaces 964 may be in abutment with portions of the PCB 652 which are devoid of any sensing traces 654A-D or excitation coil 664 portions. The one or more bias member 954 may ensure that the contact faces 964 are held firmly against the PCB 652 as the carriage 50 is displaced during operation of the syringe pump 10. Thus, the bias exerted on the paddle 950 by the at least one bias member 954 may keep the spacing of the target assembly 668 and the PCB 652 consistent during use.

Additionally, as shown best in FIG. 77, the PCB 652 may be captured within a mounting body 968 which seats within the interior of the rigid body 122. In example embodiments, the rigid body 122 is formed from a piece of extruded metal material. Such extruded metal bodies may typically have loosely controlled interior dimensions. The mounting body 968 may be arranged to substantially center the PCB 652 in the center of rigid body 122 despite loose control of the interior dimension of the extruded metal material used to form the mounting body 968.

As shown, the mounting body 968 may include a receptacle 970 within which the PCB 652 may be placed. The receptacle 970 may be defined in a central region 974 of the mounting body 968 and may be provided as a slot into which the PCB 652 may be slid during assembly. A number of stiffeners 984 (e.g. ribs) may be associated with the central region 974 to inhibit bending of the central region 974. A number of arms 972A-D may extend from the central region 974 of the mounting body 968. Some of the arms 972A, B may define guides regions 978 which may accept rails 980 formed in or coupled to the interior surface of the rigid body 122. The interaction of the guides 978 and the rails 980 may help to locate the mounting body 968 within the rigid body. Additionally, each of the arms 972A-D may be dimensioned to include two end projections 982A, B. The end projections 982A, B may contact different interior sidewalls of the rigid body 122 near a corner of the rigid body 122 formed by the sidewalls. Each end projection 982A, B may also be sized to interfere with the interior wall of the rigid body 122. Thus, when the mounting body 968 is installed within the rigid body 122, the arms 972A-D may deflect so as to allow the end projections 982A, B to be accommodated within the rigid body 122. Each of the arms 972A-D may include flexures 976 as shown. The flexures 976 may be positioned such that deflection in the mounting body 968 is concentrated in desired areas while leaving other regions of the mounting body 968 stiff. By positioning the flexures 976 as shown in FIG. 77 for example, it may help to ensure that the PCB 652 is evenly spaced from the surrounding metal of the rigid body 122. It may also help to ensure that the target assembly 668 mounted to the paddle 950 is appropriately aligned over any sensing traces 654A-D and excitation coil 664 portions on the PCB 652.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

1.-200. (canceled)

201. A linear position sensing assembly comprising:
a printed circuit board comprising:
a plurality of sensing traces including short and long period sinusoidal sensing traces, and short and long period cosinusoidal sensing traces, each of the plurality of sensing traces being doubled back at least once across a trace originating layer and 180° out of phase across a return layer of the printed circuit board that are dedicated to each sensing trace;
an excitation coil surrounding the sensing traces and having flanking regions extending along either side of the plurality of sensing traces;
a set of ferrite bodies coupled to a side of the PCB, a first of the ferrite bodies aligned with the plurality of sensing traces, a second and third of the ferrite bodies being each being aligned with one of the flanking regions;
a target assembly configured to displace in a range over the sensing traces, the target assembly including a coil and a capacitor configured to form a resonant circuit, the target assembly having a main body with a peg extending toward the sensing traces, a tip region of the peg being beveled to a thinnest width which is a fraction of the shortest period length of any of the plurality of sensing traces.

202. The linear position sensing assembly of claim 201, wherein each of the short period sinusoidal and cosinusoidal sensing traces are doubled back at least four times.

203. The linear position sensing assembly of claim 201, wherein at least the short period sinusoidal and cosinusoidal sensing traces are doubled back at least eight times.

204. The linear position sensing assembly of claim 201, wherein the short period sinusoidal and cosinusoidal sensing traces have periods of 0.1 inch or less.

205. The linear position sensing assembly of claim 201, wherein the short period sinusoidal and cosinusoidal sensing traces have periods of 2 mm or less.

206. The linear position sensing assembly of claim 201, wherein the long period sinusoidal and cosinusoidal sensing traces have a period at least equivalent to the displacement range of the target assembly.

207. The linear position sensing assembly of claim 201, wherein the excitation coil is disposed on an exterior layer of the PCB shared with a portion of one of the sensing traces.

208. The linear position sensor assembly of claim 201, wherein the main body of the target assembly is constructed of a ferrite material.

209. The linear position sensor assembly of claim 201, wherein the peg extends from a central region of a back bone of the main body and the main body includes a pair of arms on opposing sides of the peg, each arm extending over a respective flanking region of the excitation coil.

210. The linear position sensor assembly of claim 209, wherein the coil is positioned on the peg and the target assembly includes a pair of arm coils, each of the pair of arm coils being disposed around a respective arm of the pair of arms.

211. The linear position sensor assembly of claim 201, wherein the tip region of the peg is beveled to a thinnest width no more than 10% of the shortest period length of any of the plurality of sensing traces.

212. A syringe pump comprising:
a drive assembly including a drive motor, a leadscrew and a nut, the drive motor configured to engender displacement of the nut along the leadscrew when powered;
a drive head coupled to the drive assembly and configured to displace as the nut is displaced along the leadscrew;
a linear position sensing assembly comprising:
a circuit board including an excitation coil;
a set of sensing traces each doubled back at least once in antiphase on different layers of the circuit board, the sensing traces including short period sinusoidal and cosinusoidal sensing traces and long period sinusoidal and cosinusoidal sensing traces; and
a resonant target assembly coupled with the drive assembly and configured to displace in tandem with the drive head, the target assembly including a beveled projection positioned over the sensing traces; and
a controller configured to power the excitation coil to resonate the target assembly, the controller further configured to monitor an induced voltage on each of the sensing traces and determine a position of the target assembly based at least in part on the induced voltage on each sensing trace.

213. The syringe pump of claim 212, wherein the target assembly is constructed of a ferrite material and includes two arms disposed over portions of the excitation coil.

214. The syringe pump of claim 212, wherein the target assembly is coupled to a carriage which displaces in tandem with the drive head.

215. The syringe pump of claim 214, wherein the carriage displaces along at least one bearing surface on a rigid back bone of the syringe pump and the drive head is also configured to displace along at least one drive head bearing surface on the rigid back bone.

216. The syringe pump of claim 212, wherein the controller is further configured to govern displacement of the drive head of the syringe pump based at least in part upon the position of the target assembly.

217. The syringe pump of claim 212, wherein each of the short period sinusoidal and cosinusiodal sensing traces are doubled back at least four times.

218. The syringe pump of claim 212, wherein each of the short period sinusoidal and cosinusoidal sensing traces has a period of no more than 2 mm.

219. The syringe pump of claim 212, wherein the beveled projection includes a tip region which has a thinnest width no more than 30% of the shortest period length of any of the plurality of sensing traces.

220. The syringe pump of claim 212, wherein the circuit board includes a ferrite body disposed in alignment with the sensing traces.

221. The syringe pump of claim 212, wherein the excitation coil includes regions extending adjacent the sensing traces, the regions each being associated with ferrite bodies.

222. The syringe pump of claim 212, wherein the controller is configured to determine a position of the target assembly with an output resolution of less than one micron.

223. The syringe pump of claim 212, wherein the magnetic field generated as the target resonates has a thinnest region no more than 15% the shortest period length of any of the plurality of sensing traces.

224. A method of determining the position of the drive head of a syringe pump comprising:
illuminating an excitation coil of a circuit board;
resonating a resonant circuit in a target assembly having a projection with a beveled end;
receiving, with a controller, a reading of a voltage induced on each of a plurality of sensing traces positioned adjacent the beveled end and on the circuit board, the plurality of sensing traces including short period sinusoidal and cosinusoidal sensing traces and long period sinusoidal and cosinusoidal sensing traces, each sensing trace doubled back at least once in antiphase on different layers of the circuit board; and
analyzing, with a controller, the induced voltage on each of the sensing traces to determine a position of the target assembly in relation sensing traces.

225. The method of claim 224, wherein analyzing the induced voltage on each of the sensing traces comprises checking the induced voltages against a look up table.

226. The method of claim 224, wherein the method further comprises, halting illuminating the excitation coil before receiving the reading of a voltage induced on each of the plurality of sensing traces.

227. The method of claim 224, wherein analyzing the induced voltage on each of the sensing traces comprises analyzing the induced voltage from each of the long period traces to determine a particular sinusoidal period of the short period sinusoidal trace and a particular consinusoidal period of the short period cosinusoidal trace over which the beveled end is positioned.

228. The method of claim 227, wherein analyzing the induced voltage on each of the sensing traces further comprises analyzing the induced voltage from each of the short period sinusoidal and consinusoidal trace and determining the position of the target assembly.

229. The method of claim 228, wherein determining the position of the target assembly comprises determining the position of the target assembly with submicron resolution.

230. The method of claim 224, wherein the method further comprises generating a command for a drive motor of the syringe pump based at least in part upon the determined position of the target assembly.

231. The method of claim 224, wherein the method further comprises providing a ferrite body aligned with the sensing traces on the circuit board and associating additional ferrite bodies with portions of the excitation coil which are adjacent the sensing traces.

232. (canceled)
